(19) United States
(12) Patent Application Publication
(10) Pub. No.: US 2005/0208514 A1
(43) Pub. Date: Sep. 22, 2005

(54) Novel fibroblast growth factor and nucleic acids encoding dame

(76) Inventors: John Alsobrook II, Madison, CT (US); Ferenc L. Boldog, North Haven, CT (US); Michael E. Jeffers, Branford, CT (US); William J. LaRochelle, Madison, CT (US); Denise M. Lepley, Hartford, CT (US); Henri Lichenstein, Guilford, CT (US); Richard A. Shimkets, Guilford, CT (US); Meijia Yang, Scituate, MA (US); Marie Ruiz-Martinez, Bethany, CT (US); Galina Chernaya, Madison, CT (US); Muralidhara Padigaru, Malad Mumbai (IN); Sudhirdas K. Prayaga, O'Fallon, MO (US); Mei Zhong, Branford, CT (US)

Correspondence Address: Jenell Lawson;Intellectual Property, CuraGen Corporation, 555 Long Wharf Drive, New Haven, CT, 06551, US

(21) Appl. No.: 10/980,659

(22) Filed: Nov. 3, 2004

Related U.S. Application Data
(63) Continuation-in-part of application No. 10/174,394, filed on Jun. 17, 2002.
Application No. 10/174,394 is a continuation-in-part of application No. 09/817,814, filed on Mar. 26, 2001, now abandoned.
Application No. 09/817,814 is a continuation-in-part of application No. 09/609,543, filed on Jul. 3, 2000.
Application No. 09/609,543 is a continuation-in-part of application No. 09/494,585, filed on Jan. 31, 2000, now abandoned.
(60) Provisional application No. 60/145,899, filed on Jul. 27, 1999.

(51) Int. Cl. C12Q 1/68; A61K 38/18; C07K 14/50

(52) U.S. Cl. 435/6; 435/69.1; 435/320.1; 435/325; 530/399; 514/12; 536/23.5

(57) ABSTRACT

The present invention generally relates to nucleic acids, proteins, and antibodies. The invention relates more particularly to nucleic acid molecules, proteins, and antibodies of Fibroblast Growth Factor-20 (FGF-20), or its fragments, derivatives, variants, homologs, analogs, or a combination thereof.

This application is a continuation-in-part of U.S. patent application Ser. No. 10/174,394, filed Jun. 7, 2002, which is a continuation-in-part of U.S. patent application Ser. No. 09/817,814, filed Mar. 26, 2001, which is a continuation-in-part of U.S. patent application Ser. No. 09/609,543, filed Jul. 3, 2000, which is a continuation-in-part application of U.S. patent application Ser. No. 09/494,585, filed Jan. 31, 2000, which in turn claims priority to U.S. Provisional Application No. 60/145,899, filed Jul. 27, 1999. This application also claims the priority benefits of U.S. patent application Ser. No. 10/842,206, filed May 10, 2004. The contents of each of these applications are incorporated herein by reference in their entireties.

### 1. FIELD OF THE INVENTION

The present invention generally relates to nucleic acids, proteins, and antibodies. The invention relates more particularly to nucleic acid molecules, proteins, and antibodies of Fibroblast Growth Factor-20 (FGF-20), or its fragments, derivatives, variants, homologs, analogs, or a combination thereof.

### 2. BACKGROUND OF THE INVENTION

The fibroblast growth factor (“FGF”) family has more than 20 members. Previously described members of the FGF family regulate diverse cellular functions such as growth, survival, apoptosis, motility and differentiation (Szebenyi & Fallon (1999) Int. Rev. Cytol. 185, 45-106). These molecules transduce signals intracellularly via high affinity interactions with cell surface tyrosine kinase FGF receptors (FGFRs), four of which have been identified (Xu, X., Weinstein, M., Li, C. & Deng, C. (1999) Cell Tissue Res. 296, 33-43; Klint, P. & Claesson-Welsh, L. (1999) Front. Biosci. 4, 165-177). These FGF receptors are expressed on most types of cells in tissue culture. Dimerization of FGF receptor monomers upon ligand binding has been reported to be a requisite for activation of the kinase domains, leading to receptor trans phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

FGFs also bind, albeit with low affinity, to heparin sulfate proteoglycans (HSPGs) present on most cell surfaces and extracellular matrices (ECM). Interactions between FGFs and HSPGs serve to stabilize FGF/FGFR interactions, and to sequester FGFs and protect them from degradation (Szebenyi, G. & Fallon, J. F. (1999)). Due to its growth-promoting capabilities, one member of the FGF family, FGF-7, is currently in clinical trials for the treatment of chemotherapy-induced mucositis (Danilenko, D. M. (1999) Toxicol. Pathol. 27, 64-71).

In addition to participating in normal growth and development, known FGFs have also been implicated in the generation of pathological states, including cancer (Basilico, C & Moscatelli, D. (1992) Adv. Cancer Res. 59, 115-165). FGFs may contribute to malignancy by directly enhancing the growth of tumor cells. For example, autocrine growth stimulation through the co-expression of FGF and FGFR in the same cell leads to cellular transformation (Matsumoto-Yoshitomi, et al. (1997) Int. J. Cancer 71, 442-450). Likewise, the constitutive activation of FGFR via mutation or rearrangement leads to uncontrolled proliferation (Lorenzi, et al. (1996) Proc. Natl. Acad. Sci. USA. 93, 8956-8961; Li, et al. (1997) Oncogene 14,1397-1406). Furthermore, some FGFs are angiogenic (Gerwins, et al. (2000) Crit. Rev. Oncol. Hematol. 34,185-194). Such FGFs may contribute to the tumorigenic process by facilitating the development of the blood supply needed to sustain tumor growth. Not surprisingly, at least one FGF is currently under investigation as a potential target for cancer therapy (Gasparini (1999) Drugs 58, 17-38).

Expression of FGFs and their receptors in the brains of perinatal and adult mice has been examined. Messenger RNA all FGF genes, with the exception of FGF-4, is detected in these tissues. FGF-3, FGF-6, FGF-7 and FGF-8 genes demonstrate higher expression in the late embryonic stages than in postnatal stages, suggesting that these members are involved in the late stages of brain development. In contrast, expression of FGF-1 and FGF-5 increased after birth. In particular, FGF-6 expression in perinatal mice has been reported to be restricted to the central nervous system and skeletal muscles, with intense signals in the developing cerebrum in embryos but in cerebellum in 5-day-old neonates. FGF-receptor (FGFR)-4, a cognate receptor for FGF-6, demonstrate similar spatiotemporal expression, suggesting that FGF-6 and FGFR-4 plays significant roles in the maturation of nervous system as a ligand-receptor system. According to Ozawa et al., these results strongly suggest that the various FGFs and their receptors are involved in the regulation of a variety of developmental processes of brain, such as proliferation and migration of neuronal progenitor cells, neuronal and glial differentiation, neurite extensions, and synapse formation.

Other members of the FGF polypeptide family include the FGF receptor tyrosine kinase (FGFRTK) family and the FGF receptor heparin sulfate proteoglycan (FGFRHS) family. These members interact to regulate active and specific FGFR signal transduction complexes. These regulatory activities are diversified throughout a broad range of organs and tissues, and in both normal and tumor tissues, in mammals. Regulated alternative messenger RNA (mRNA) splicing and combination of variant subdomains give rise to diversity of FGFRTK monomers. Divalent cations cooperate with the FGFRHS to conformationally restrict FGFRTK trans-phosphorylation, which causes depression of kinase activity and facilitates appropriate activation of the FGFR complex by FGF. For example, it is known that different point mutations in the FGFRTK commonly cause craniofacial and skeletal abnormalities of graded severity by graded increases in FGF-independent activity of total FGFR complexes. Other processes in which FGF family exerts important effects are liver growth and function and prostate tumor progression.

Glia-activating factor (GAF), another FGF family member, is a heparin-binding growth factor that was purified from the culture supernatant of a human glioma cell line. See, Miyamoto et al. 1993, Mol Cell Biol 13(7): 4251-4259. GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N terminus like those in acidic FGF and basic FGF.

Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N terminus with respect to those predicted by the cDNA sequence, except the initiation methionine. The rat FGF 9 cDNA was also cloned, and the structural analysis indicated that the FGF-9 gene is highly conserved.

Citation or discussion of a reference herein shall not be construed as an admission that such is prior art to the present invention.

### 3. SUMMARY OF THE INVENTION

The present invention is based, in part, upon the discovery of a nucleic acid encoding a novel polypeptide having homology to members of the Fibroblast Growth Factor (FGF) family of proteins. The present invention provides nucleic acids and proteins (including peptides and polypeptides) of FGF-20, its variants, derivatives, homologs, and analogs (collectively referred as “CG53135”). The present invention also provides antibodies against a CG53135 protein.

In one aspect, the invention provides an isolated CG53135 protein. In some embodiments, the isolated protein comprises the amino acid sequence of SEQ ID NO:2. In other embodiments, the invention includes a variant of SEQ ID NO:2, in which some amino acids residues, e.g., no more than 1%, 2%, 3%, 5%, 10% or 15% of the amino acid sequences of SEQ ID NO;2 are changed. In some embodiments, the isolated FGF-20 protein comprise the amino acid sequence of a mature form of an amino acid sequence given by SEQ ID NO:2, or a variant of a mature form of an amino acid sequence given by SEQ ID NO:2. Preferably, no more than 1%, 2%, 3%, 5%, 10% or 15% of the amino acid sequences of SEQ ID NO;2 are changed in the variant of the mature form of the amino acid sequence.

In another aspect, the invention provides a fragment of an FGF-20 protein, including fragments of variant FGF-20 proteins, mature FGF-20 proteins, and variants of mature FGF-20 proteins, as well as FGF-20 proteins encoded by allelic variants and single nucleotide polymorphisms of FGF-20 nucleic acids. An example of an FGF-20 protein is a fragment that includes residues 3-211, 9-211, 12-211, 15-211, 24-211, 54-211, or 55-211 of FGF-20 (SEQ ID NO:2).

In another aspect, the invention includes an isolated CG53135 nucleic acid molecule. The CG53135 nucleic acid molecule can include a sequence encoding any of the FGF-20 proteins, variants, or fragments disclosed above, or a complement to any such nucleic acid sequence. In one embodiment, the sequences include those disclosed in SEQ ID NO:1, 3, 5, 6, 8, 9, 11, 13, 15, 17, 19, 21, 23, 25, 27, 29, 31, 33, 35, 37, 39, or 41. In other embodiments, the FGF-20 nucleic acids include a sequence wherein nucleotides different from those given in SEQ ID NO:1 may be incorporated. Preferably, no more than 1%, 2%, 3,%, 5%, 10%, 15%, or 20% of the nucleotides are so changed.

In one embodiment, the nucleic acid encodes a protein fragment that includes residues 2-211, 3-211, 9-211, 12-211, 15-211,24-211, 54-211, or 55-211 of SEQ ID NO:2. The nucleic acid can include, e.g., nucleotides 163-633 of SEQ ID NO:1 or nucleotides 70-633 of SEQ ID NO:1.

In other embodiments, the invention includes fragments or complements of these nucleic acid sequences. Vectors and cells incorporating CG53135 nucleic acids are also included in the invention. The present invention further provides methods of isolating a CG53135 protein by culturing the host cells containing a CG53135 nucleic acid in a suitable nutrient medium, and isolating one or more expressed CG53135 proteins. In a preferred embodiment, the host cell is E. coli.

In another embodiment, the present invention provides a method of stimulating proliferation, differentiation or migration of epithelial cells and/or mesenchymal cells comprising administering to a subject in need thereof an effective amount of a composition comprising one or more CG53135 proteins or nucleic acids. In a specific embodiment, the epithelial cells or mesenchymal cells are locate at the alimentary tract or pulmonary tract (e.g., trachea) of the subject.

The invention also includes antibodies that bind immunospecifically to any of the CG53135 proteins described herein. The CG53135 antibodies in various embodiments include, e.g., polyclonal antibodies, monoclonal antibodies, humanized antibodies and/or human antibodies.

The invention additionally provides pharmaceutical compositions that include a CG53135 protein, a CG53135 nucleic acid or a CG53135 antibody of the invention. Also included in the invention are kits that include, e.g., a CG53135 protein, a CG53135nucleic acid or a CG53135antibody.

Several methods are included in the invention. For example, a method is disclosed for determining the presence or amount of a CG53135 protein in a sample of animal or human serum or plasma. The method includes capturing CG53135 proteins with an immobilized monoclonal antibody to CG53135, addition of a rabbit secondary polyclonal antibody to CG53135 and detecting the rabbit antibody with donkey-anti-rabbit-horseradish peroxidase conjugate using standard ELISA techniques.

Similarly, the invention discloses a method for determining the presence or amount of a CG53135 nucleic acid molecule in a sample. The method includes contacting the sample with a probe that binds to the nucleic acid molecule; and determining the presence or amount of the probe bound to the nucleic acid molecule, such that the probe indicates the presence or amount of the CG53135 nucleic acid molecule in the sample.

Also provided by the invention is a method for identifying an agent that binds to a CG53135 protein. The method includes determining whether a candidate substance binds to a CG53135 protein. Binding of a candidate substance indicates the agent is an CG53135 protein binding agent.

The invention also includes a method for identifying a potential therapeutic agent for use in treatment of a pathology. The pathology is, e.g., related to aberrant expression, aberrant processing, or aberrant physiological interactions of a CG53135 protein of the invention. This method includes providing a cell which expresses the CG53135 protein and has a property or function ascribable to the protein; contacting the provided cell with a composition comprising a candidate substance; and determining whether the substance alters the property or function ascribable to the protein, in comparison to a control cell. Any such substance is identified as a potential therapeutic agent. Furthermore, therapeutic agents may be identified by subjecting any potential therapeutic agent identified in this way to additional tests to identify a therapeutic agent for use in treating the pathology.

In some embodiments, the property or function relates to cell growth or cell proliferation, and the substance binds to the protein, thereby modulating an activity of the protein. In some embodiments, the candidate substance has a molecular weight not more than about 1500 Da. In some embodiments, the candidate substance is an antibody. The invention additionally provides any therapeutic agent identified using a method such as those described herein.

The invention also includes a method for screening for a modulator of latency or predisposition to a disorder associated with aberrant expression, aberrant processing, or aberrant physiological interactions of a CG53135 protein. The method includes providing a test animal that recombinantly expresses the CG53135 protein of the invention and is at increased risk for the disorder; administering a test compound to the test animal; measuring an activity of the protein in the test animal after administering the compound; and comparing the activity of the FGF-20 protein in the test animal with the activity of the CG53135 protein in a control animal not administered the compound. If there is a change in the activity of the protein in the test animal relative to the control animal, the test compound is a modulator of latency of or predisposition to the disorder.

The invention also provides a method for determining the presence of or predisposition to a disease associated with altered levels of a CG53135 protein or of a CG53135 nucleic acid of the invention in a first mammalian subject. The method includes measuring the level of expression of the protein or the amount of the nucleic acid in a sample from the first mammalian subject; and comparing its amount in the sample to its amount present in a control sample from a second mammalian subject known not to have, or not to be predisposed to, the disease. An alteration in the expression level of the protein or the amount of the nucleic acid in the first subject as compared to the control sample indicates the presence of or predisposition to the disease.

Also provided by the invention is a method of treating a pathological state in a mammal, wherein the pathology is related to aberrant expression, aberrant processing, or aberrant physiological interactions of a CG53135 protein of the invention. The method includes administering to the mammal a protein of the invention in an amount that is sufficient to alleviate the pathological state, wherein the CG53135 protein is a protein having an amino acid sequence at least 85%, 90%, 95%, 96%, 97%, 98%, or even 99% identical to a protein comprising an amino acid sequence of SEQ ID NO:2, or a biologically active fragment thereof. In another related method, an antibody of the invention is administered to the mammal.

In another aspect, the invention, the invention includes a method of promoting growth of cells in a subject. The method includes administering to the subject a CG53135 protein of the invention in an amount and for a duration that are effective to promote cell growth. In some embodiments, the subject is a human, and the cells whose growth is to be promoted may be chosen from among cells in the vicinity of a wound, cells in the vascular system, cells involved in hematopoiesis, cells involved in erythropoiesis, cells in the lining of the gastrointestinal tract, and cells in hair follicles.

In a further aspect, the invention provides a method of inhibiting growth of cells in a subject, wherein the growth is related to expression of a CG53135 protein of the invention. This method includes administering to the subject a composition that inhibits growth of the cells. In a one embodiment, the composition includes an antibody of the invention. Significantly, the subject is a human, and the cells whose growth is to be inhibited are chosen from among transformed cells, hyperplastic cells, tumor cells, and neoplastic cells.

In a still further aspect, the invention provides a method of treating or preventing or delaying a tissue proliferation-associated disorder. The method includes administering to a subject in which such treatment or prevention or delay is desired a CG53135 antibody in an amount sufficient to treat, prevent, or delay a tissue proliferation-associated disorder in the subject.

The tissue proliferation-associated disorders diagnosed, treated, prevented or delayed using the CG53135 nucleic acid molecules, proteins or antibodies can involve epithelial cells, e.g., fibroblasts and keratinocytes in the anterior eye after surgery. Other tissue proliferation-associated disorders include, e.g., tumors, restenosis, psoriasis, Dupuytren's contracture, diabetic complications, Kaposi sarcoma, and rheumatoid arthritis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### 4. BRIEF DESCRIPTION OF THE DRAWINGS

FIG. 1 shows a Western analysis of FGF-20. Samples from 293 cells (Panel A) or NIH 3T3 cells (Panel B) transiently transfected with the indicated construct were examined by Western analysis using anti-V5 antibody. CM=conditioned media, SE=suramin-extracted conditioned media. Molecular mass markers are indicated on the left.

FIG. 2 shows a Western analysis of FGF-20 protein secreted by 293 cells.

FIG. 3 shows a Western analysis of FGF-20 (SEQ ID NO:2) protein expressed in E. coli cells.

FIG. 4 presents an analysis of the expression of FGF-20 obtained by real-time quantitative PCR using FGF-20-specific TaqMan reagents. Results for normalized RNA derived from normal human tissue samples are shown in Panel A, and from tumor cell lines in Panel B. Results obtained using tumor tissues obtained directly during surgery are shown in Panels C and D.

FIG. 5 displays the biological activity of recombinant FGF-20 as represented by its effects on DNA synthesis. Cells were serum-starved, incubated with the indicated factor for 18 hours, and analyzed by a BrdU incorporation assay. Samples were performed in triplicate. Panel A, NIH 3T3 mouse fibroblasts. Panel B, CCD-1070 human fibroblasts. Panel C, CCD-1106 human keratinocytes

FIG. 6 presents an image of a Coomassie Blue stained SDS-PAGE gel of purified samples of FGF-20 prepared under reducing and nonreducing conditions.

FIG. 7 shows the results of experiments assessing the receptor binding specificity of FGF-20. NIH 3T3 cells were serum-starved, incubated with the indicated growth factor (square=PDGF-BB; triangle=aFGF; circle=FGF-20) either alone or together with the indicated soluble FGFR, and analyzed by a BrdU incorporation assay. Experiments were performed in triplicate and are represented as the percent BrdU increase in incorporation of BrdU relative to cells receiving the growth factor alone.

FIG. 8 shows an image of a Coomassie Blue stained SDS-PAGE gel of the arginine supernatant obtained when plasmid pET24a- FGF20X-del54-codon was expressed in E. coli strain BL21 (DE3).

FIG. 9 displays the biological activity of a truncated form of recombinant FGF-20 (CG53135-17, denoted by (d1-23)FGF20 in the Figure) as represented by its effects on DNA synthesis, compared to that of full length FGF-20 (denoted FGF20 in the Figure). NIH 3T3 mouse fibroblasts were serum-starved, incubated with the indicated factor for 18 hours, and analyzed by a BrdU incorporation assay.

FIG. 10 shows liquid chromatography and mass spectrometry analysis of CG53135-05 E. coli purified product. CG53135-05 E. coli purified product was injected onto the phenyl-hexyl column in an aqueous mobile phase containing 95% water, 5% acetonitrile, and 0.1% trifluoroacetic add. The protein was then eluted by using a non-linear gradient with an organic mobile phase containing 95% acetonitrile, 5% water, and 0.085% trifluoroacetic acid. Each of the 4 peaks was characterized using LC/ESI/MS, MALDI-TOF MS, and N-terminal amino acid sequencing.

FIGS. 11A and 11B depict peptide map of CG53135-05 E. coli purified product. The upper tracing in each panel represents that of CG53135 and the lower tracing in each panel represents an identical sample treated similarly but without CG53135. FIG. 2A: detection at 214 nm to monitor CG53135 protein. FIG. 11B: detection at 295 nm to monitor tryptophan-containing protein.

FIG. 12 shows the effect of CG53135 in the closure of wounds in various human cell lines.

FIG. 13 shows CG53135 induces DNA synthesis in NIH 3T3 murine embryonic lung fibroblasts.

FIG. 14 shows CG53135 sustaines NIH 3T3 cell growth.

FIG. 15 shows CG53135 induces DNA synthesis in the 786-O human renal carcinoma cell line in a dose-dependent manner.

FIG. 16(A) shows scan of Tris-glycine SDS gel analysis of fractions (bleed 1-2, rabbit #2448) from purified IgG under reducing conditions. Lane 1, empty; lane 2, crude bleed 1 IgG 1:5; lane 3, empty; lane 4, MK12; lane 5, E1 (eluate #1, bleed 1); lane 6, E2 bleed 1; lane 7, E3 bleed 1; lane 8, E4 bleed 1; lane 9, E5 bleed 1; lane 10, E1 bleed 2; lane 11, E2 bleed 2; lane 12, E3 bleed 2; lane 13, E4 bleed 2; lane 14, E5 bleed 2; lane 15, Mark 12 molecular weight standards. (B) shows scan of Tris-glycine SDS gel analysis of fractions (bleed 3-4, rabbit #2448) from purified IgG under reducing conditions. Lane 1, empty; lane 2, crude bleed 2 IgG 1:5; lane 3, empty (slight contamination from lane 4); lane 4, MK12; lane 5, E1 (eluate #1, bleed 3); lane 6, E2 bleed 3; lane 7, E3 bleed 3; lane 8, E4 bleed 3; lane 9, E5 bleed 3; lane 10, E1 bleed 4; lane 11, E2 bleed 4; lane 12, E3 bleed 4; lane 13, E4 bleed 4; lane 14, E5 bleed 4; lane 15, Mark 12 molecular weight standards.

FIG. 17 shows the cell positions in the crypt. The bottom of the crypt is cell position 1, the crypt base. In the small intestine, the stem cells are located around cell position 4 and the proliferative cells occupy roughly half of the crypt. The cells are constantly maturing such that the cells are fully differentiated and not cycling at the top of the crypt. Changes that may affect stem cells versus their transit amplifying daughter cells can be detected by examining changes in event (labeling, apoptosis, mitosis, etc.) frequency at each cell position.

FIG. 18 shows a survival curve of intestinal crypt cells from mice prophylactically administered CG53135 or PBS, following different radiation dosages.

FIG. 19 shows the effect of prophylactic administration of CG53135 on mice intestinal crypt survival after radiation insult.

FIG. 20 shows the effect of CG53135 multiple-dose administration prior to irradiation on crypt survival curves. Animals (n=6/group) were administered PBS or CG53135-05 E. coli purified product (12 mg/kg) by intraperitoneal (IP) injection once daily for 4 consecutive days prior to a single 10, 11, 12, 13, or 14 Gy dose of X-ray whole-body irradiation on Day 0. The plot represents the radiation dose-response for crypt survival. Data points represent crypt survival in individual animals analyzed using a multi-target (Puck) analysis model, DRFIT.

FIG. 21 shows effect of CG53135 on repopulation of thymus following bone marrow ablation and subsequent bone marrow transplant.

### 5. DETAILED DESCRIPTION OF THE INVENTION

This invention is based, in part, on the discovery of a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs. This class of proteins and/or nucleic acid molecules is designated as “CG53135.” CG53135 can stimulate proliferation of epithelial cells and/or mesenchymal cells in vivo, and thus have variety of uses, such as promoting wound healing.

For clarity of disclosure, and not by way of limitation, the detailed description of the invention is divided into the following subsections:
(i) CG53135
(ii) Methods of Preparing CG53135
(iii) Antibodies to CG53135
(iv) Structure Prediction and Functional Analysis of CG53135
(v) Uses of CG53135
(vi) Administration, Pharmaceutical Compositions and Kits

### 5.1. CG53135

The present invention provides nucleic acid molecules encoding FGF-20, or its fragments, derivatives, variants, homologs, or analogs, and the proteins (including peptides and polypeptides) encoded by such nucleic acid molecules. Such nucleic acid molecules and the proteins are collectively termed as “CG53135.” The present invention further provides antibodies against a CG53135 protein, and methods of use for CG53135 as well as antibodies against a CG53135 protein.

As used herein, the term “CG53135” refers to a class of proteins or nucleic acids encoding such proteins or their complementary strands, where the proteins comprise an amino acid sequence of SEQ ID NO:2 (211 amino acids), or its fragments, derivatives, variants, homologs, or analogs. In a preferred embodiment, a CG53135 protein retains at least some biological activity of FGF-20. As used herein, the term “biological activity” means that a CG53135 protein possesses some but not necessarily all the same properties of (and not necessarily to the same degree as) FGF-20.

A member (e.g., a protein and/or a nucleic acid encoding the protein) of the CG53135 family may further be given an identification name. For example, CG53135-01 (SEQ ID NOs:1 and 2) represents the first identified FGF-20; CG53135-05 (SEQ ID NOs:8 and 2) represents a codon-optimized, full length FGF-20 (i.e., the nucleic acid sequence encoding FGF-20 has been codon optimized, but the amino acid sequence has not been changed from the originally identified FGF-20). Some members of the CG53135 family may differ in their nucleic acid sequences but encode the same CG53135 protein, e.g., CG53135-01, CG53135-03, and CG53135-05 all encode the same CG53135 protein. An identification name may also be an in-frame clone (“IFC”) number, for example, IFC 250059629 (SEQ ID NOs:33 and 34) represents amino acids 63-196 of the full length FGF-20 (cloned in frame in a vector). Table 1 shows a summary of some of the CG53135 family members. In one embodiment, the invention includes a variant of FGF-20 protein, in which some amino acids residues, e.g., no more than 1%, 2%, 3%, 5%, 10% or 15% of the amino acid sequence of FGF-20 (SEQ ID NO:2), are changed. In another embodiment, the invention includes nucleic add molecules that can hybridize to FGF-20 under stringent hybridization conditions.

TABLE 1
Summary of some of the CG53135 family members

| | SEQ ID NO | |
|---|---|---|
| Name | (DNA/Protein) | Brief Description |
| CG53135-01 | 1 and 2 | FGF-20 wild type, stop codon |
| | | removed |
| CG53135-02 | 3 and 4 | Codon optimized, amino acids 2-54 |
| | | (as numbered in SEQ ID NO: 2) |
| | | were removed |
| CG53135-03 | 5 and 2 | FGF-20 wild type |
| CG53135-04 | 6 and 7 | Amino acids 20-51 (as numbered |
| | | in SEQ ID NO: 2) were removed, |
| | | also valine at position 85 is |
| | | changed to alanine |
| | | (“⁸⁵V→A”) |
| CG53135-05 | 8 and 2 | Codon optimized, full length |
| | | FGF-20 |
| CG53135-06 | 9 and 10 | Amino acids 20-51 (as numbered |
| | | in SEQ ID NO: 2) were removed |
| CG53135-07 | 11 and 12 | Protein consisting of amino acids |
| | | 1-18 (as numbered in SEQ ID |
| | | NO: 2) |
| CG53135-08 | 13 and 14 | Protein consisting of amino acids |
| | | 32-52 (as numbered in SEQ ID |
| | | NO: 2) |
| CG53135-09 | 15 and 16 | Protein consisting of amino acids |
| | | 173-183 (as numbered in SEQ ID |
| | | NO: 2) |
| CG53135-10 | 17 and 18 | Protein consisting of amino acids |
| | | 192-211 (as numbered in SEQ ID |
| | | NO: 2) |
| CG53135-11 | 19 and 20 | Protein consisting of amino acids |
| | | 121-137 (as numbered in SEQ ID |
| | | NO: 2) |
| CG53135-12 | 21 and 22 | FGF-20 SNP, aspartic acid at |
| | | position 206 is changed to |
| | | asparagines (“²⁰⁶D→N”) as |
| | | compared to CG53135-01 |
| CG53135-13 | 23 and 24 | CG53135-05 minus first 2 amino |
| | | acids at the N-terminus |
| CG53135-14 | 25 and 26 | CG53135-05 minus first 8 amino |
| | | acids at the N-terminus |
| CG53135-15 | 27 and 28 | CG53135-05 minus first 11 amino |
| | | acids at the N-terminus |
| CG53135-16 | 29 and 30 | CG53135-05 minus first 14 amino |
| | | acids at the N-terminus |
| CG53135-17 | 31 and 32 | CG53135-05 minus first 23 amino |
| | | acids at the N-terminus |
| IFC 250059629 | 33 and 34 | In frame clone, open reading |
| | | frame comprising a nucleotide |
| | | sequence encoding amino acids |
| | | 63-196 of FGF-20 (SEQ ID |
| | | NO: 2) |
| IFC 250059669 | 35 and 36 | In frame clone, open reading |
| | | frame comprising a nucleotide |
| | | sequence encoding amino acids |
| | | 63-211 of FGF-20 (SEQ ID |
| | | NO: 2) |
| IFC 317459553 | 37 and 38 | In frame clone, open reading |
| | | frame comprising a nucleotide |
| | | sequence encoding amino acids |
| | | 63-194 of FGF-20 (SEQ ID |
| | | NO: 2) with ¹⁵⁹G→E |
| IFC 317459571 | 39 and 40 | In frame clone, open reading |
| | | frame comprising a nucleotide |
| | | sequence encoding amino acids |
| | | 63-194 of FGF-20 (SEQ ID |
| | | NO: 2) |
| IFC 250059596 | 41 and 10 | In frame clone, open reading |
| | | frame comprising a nucleotide |
| | | sequence encoding amino acids |
| | | 1-19 and 52-211 of |
| | | FGF-20 (SEQ ID NO: 2) |
| IFC 316351224 | 41 and 10 | In frame clone, open reading |
| | | frame comprising a nucleotide |
| | | sequence encoding amino acids |
| | | 1-19 and 52-211 of |
| | | FGF-20 (SEQ ID NO: 2). |

As used herein, the term “FGF-20” refers to a protein comprising an amino acid sequence of SEQ ID NO:2, or a nucleic acid sequence encoding such a protein or the complementary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the term “effective amount” refers to the amount of a therapy (e.g., a composition comprising a CG53135 protein) which is sufficient to reduce and/or ameliorate the severity and/or duration of a disease or one or more symptoms thereof, prevent the advancement of a disease, cause regression of a disease, prevent the recurrence, development, or onset of one or more symptoms associated with a disease, or enhance or improve the prophylactic or therapeutic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. In a certain embodiment, the subject is a mammal, preferably a human, who has been exposed to or is going to be exposed to an insult that may induce alimentary mucositis (such as radiation, chemotherapy, or chemical warfare agents). In another embodiment, the subject is a farm animal (e.g., a horse, pig, or cow) or a pet (e.g., a dog or cat) that has been exposed to or is going to be exposed to a similar insult. The term “subject” is used interchangeably with “patient” in the present invention.

### 5.1.1. Identification of FGF-20

FGF-20 coding sequence was identified by sequencing human genomic DNAs. The DNA sequence of FGF-20 has 633 bases that encode a polypeptide predicted to have 211 amino acid residues (SEQ ID NO:2). The predicted molecular weight of FGF-20, based on the amino acid sequence, is 23498.4 Da.

The FGF-20 nucleic acid sequence was used as a query nucleotide sequence in a BLASTN search to identify related nucleic acid sequences. The FGF-20 nucleotide sequence has a high similarity to murine fibroblast growth factor 9 (FGF-9) (392 of 543 bases identical, or 72%; GenBank Accession Number S82023) and to human DNA encoding glia activating factor (GAP) (385 of 554 bases identical, or 69%; GenBank Accession Number E05822, also termed FGF-9). In addition, FGF-20 was found to have a comparable degree of identity (311 of 424 bases identical, or 73%) to a GAF sequence disclosed by Naruo et al. in Japanese Patent: JP 1993301893 entitled “Glia-Activating Factor And Its Production”.

To verify that the open reading frame (ORF) identified by genomic mining was correct, PCR amplification was used to obtain a cDNA corresponding to the predicted genomic clone. The nucleotide sequence of the obtained product precisely matches that of the predicted gene (see Example 1).

The protein encoded by the cDNA is most closely related to Xenopus FGF-20X (designated XFGF-20 or XFGF-20X herein), as well as to human FGF-9 and human FGF-16 (80%, 70% and 64% amino acid identity, respectively). Based on the strong homology with XFGF-20, the gene identified in the present invention is believed to represent its human ortholog, and is named FGF-20 herein.

In addition, amino acid residues that are conserved among FGF family members are predicted to be less amenable to alteration. For example, FGF-20 proteins of the present invention can contain at least one domain that is a typically conserved region in FGF family members, i.e., FGF-9 and XFGF-20 proteins, and FGF-20 homologs. Other amino acid residues, however, (e.g., those that are not conserved or only semi conserved among members of the FGF proteins) may not be as essential for activity and thus are more likely to be amenable to alteration.

FGF-9 sequences of three species (human, murine, and rat) have 147 of 208 residues identical with FGF-20 (SEQ ID NO:2), for an overall sequence identity of 70%. In addition, 170 of 208 residues are homologous to the sequence of FGF-20 (SEQ ID NO:2), for an overall percentage of homology of 81%.

The full length FGF-20 polypeptide (SEQ ID NO:2) was also aligned by BLASTX with Xenopus XFGF-20. FGF-20 has 170 of 211 (80%) identical residues, and 189 of 211 (89%) homology compared with Xenopus XFGF-20. Xenopus XFGF-20 was obtained from a cDNA library prepared at the tailbud stage using the product of degenerate PCR performed with primers based on mammalian FGF-9s as a probe. See, Koga et al., 1999 Biochem Biophys Res Commun 261(3):756-765. The deduced 208 amino acid sequence of the XFGF-20 open reading frame contains a motif characteristic of the FGF family. XFGF-20 has a 73.1% overall similarity to XFGF-9 but differs from XFGF-9 in its amino-terminal region (33.3% homology). This resembles the similarity seen for the presently disclosed SEQ ID NO:2 with respect to various mammalian FGF-9 and FGF-16 sequences, including human.

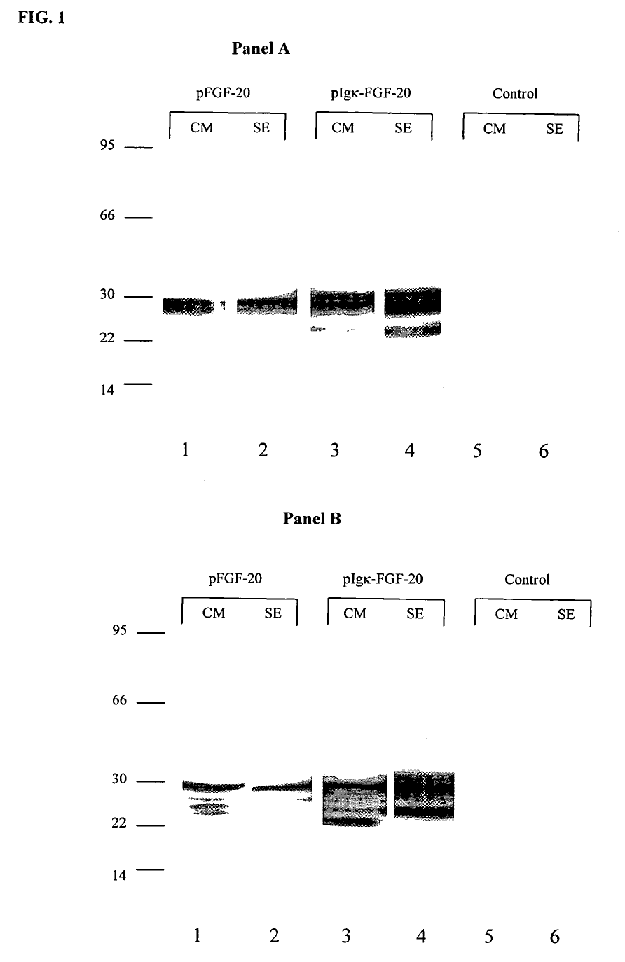

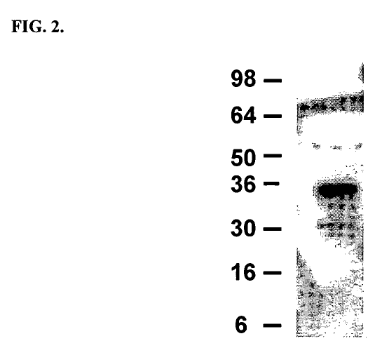

FGF-20 lacks a classical amino-terminal signal sequence as predicted by PSORT (Nakai, K & Kanehisa, M. (1992) Genomics 14, 897-911) and SIGNALP (Nielsen, et al. (1997) Protein Eng. 10, 1-6) computer algorithms, just as found for some of its closest human family members (e.g. FGF-9 and FGF-16). Nonetheless, both FGF-9 and FGF-16 are secreted (Matsumoto-Yoshitomi, et al. (1997) Int. J. Cancer 71, 442-450; Miyake, et al. (1998) Biochem. Biophys. Res. Comm. 243,148-152; Miyakawa, et al. (1999) J. Biol. Chem. 274, 29352-29357; Revest et al. (2000) J Biol. Chem. 275, 8083-8090). To determine whether FGF-20 is also secreted, the cDNA encoding the full length FGF-20 protein was subcloned into a mammalian expression vector designated pFGF-20. The protein expressed when human embryonic kidney 293 cells are transfected with this vector is found in the conditioned medium, and exhibits a band detected by an antibody to a C-terminal V5 epitope, with an apparent molecular weight in a Western blot of ˜27 kDa (FIG. 1). An additional portion of the expressed protein is released from sequestration on the 293 cells by treatment with a substance that inhibits interaction with heparin sulfate proteoglycan (HSPG). The protein released in this way also exhibits a similar Western blot pattern (FIG. 1). Similarly, when the protein is expressed in HEK293 cells from a recombinant plasmid incorporating an Ig Kappa signal sequence, a band is detected by Western blot with an apparent molecular weight of approximately 34 kDa (FIG. 2).

ClustalW multiple protein alignments (Thompson, et al. (1994) Nucleic Acids Res. 22, 4673-4680) for several vertebrate FGF-like proteins, including the FGF-20 of the present invention indicate that the three mammalian proteins resemble each other very closely but differ considerably from the FGF-20 protein of the present invention (SEQ ID NO:2). Also, the Xenopus XFGF-20 and the sequence of SEQ ID NO:2 resemble each other more closely than those of FGF-9. The internal hydrophobic domain involved in FGF-9 secretion (see, e.g., Miyakawa, et al. (1999) J. Biol. Chem. 274, 29352-29357) spans residues 95-120 of the FGF-9 sequence. Software for determining a hydropathy plot of FGF-20 are well known in the art, including, for example, the Kyte Doolittle, and other algorithms further described below.

The expression of Xenopus XFGF-20 and of Xenopus FGF-9 are distinct from each other. XFGF-20 mRNA is expressed in diploid cells, in embryos at and after the blastula stage, and specifically in the stomach and testis of adults; whereas XFGF-9 mRNA is expressed maternally in eggs and in many adult tissues. Correct expression of XFGF-20 during gastrulation appears to be required for the formation of normal head structures in Xenopus laevis. When XFGF-20 mRNA was overexpressed in early embryos, gastrulation was abnormal and development of anterior structures was suppressed. In such embryos, expression of the Xbra transcript, among those tested, was suppressed during gastrulation, indicating that expression of the Xbra gene mediates XFGF-20 effects.

The expression patterns of the related XFGF-9 polypeptide in proliferating tissues, (including, e.g., ova, testis, stomach, and multiple tissues in the maternal frog), suggests a role for XFGF-20 in the maintenance of tissues that normally undergo regeneration in a functioning organism.

It is shown in Example 8 that FGF-20 mRNA of the invention is expressed in normal cerebellum, as well as in several human tumor cell lines including carcinomas of the lung, stomach and colon but not in the corresponding normal tissues. The lack of FGF-20 expression in normal lung, stomach and colon, and its presence in tumor lines from these tissues, indicates that these cancer cell lines apparently overexpress FGF-20 in an inappropriate fashion. The chromosomal region to which FGF-20 maps is commonly altered in colorectal, lung and gastric carcinomas (Emi, et al. (1992) Cancer Res. 52, 5368-5372; Baffa, et al. (2000) Clin. Cancer Res. 6, 1372-1377). It is possible that the establishment of an FGF-20-driven autocrine growth loop in these cells contributes to their initial tumorigenic conversion and/or to their subsequent expansion.

### 5.1.2. FGF-20 Derivatives, Variants, Homologs, Analogs and Fragments

The present invention also provides derivatives, variants, homologs, analogs and fragments of FGF-20. For example, Section 6, infra, describes identification and cloning of additional FGF-20 variants. BLASTN and BLASTP analyses were performed for, e.g., CG53135-02 and CG53135-06. FGF-20 protein is predicted by the program PSORT to have high probabilities for sorting through the membrane of the endoplasmic reticulum and of the microbody (peroxisome). The CG53135-02 and CG53135-06 variant polypeptides are predicted by PSORT to have a probability of 0.8500 to be in the endoplasmic reticulum (membrane). In alternative embodiments, the CG53135-02 and CG53135-06 variant polypeptides are located in the plasma membrane with a probability of 0.7900, a microbody (peroxisome) with a probability of 0.7478 or the mitochondrial inner membrane with a probability of 0.100. The CG53135-02 and CG53135-06 variant polypeptides are predicted by the software program INTEGRAL to have a -6.42 likelihood of being a transmembrane domain between amino acid residues 62-78 (60-81). The FGF-20 polypeptide seems to be a type II (Ncyt Cexo) membrane protein.

In addition, although it does not have a predicted known cleavable signal sequence at its N-terminus, a hydropathy plot of the protein shows that FGF-20 has a prominent hydrophobic segment at amino acid positions about 90 to about 115. This single hydrophobic region is known to be a sorting signal in other members of the FGF family. Accordingly, a polypeptide that includes such amino acids is useful as a sorting signal, allowing secretion through various cellular membranes, such as the endoplasmic reticulum, the Golgi membrane or the plasma membrane. A hydropathy plot of the CG53135-02 and CG53135-06 variant proteins indicates that two prominent hydrophobic segments reside at amino acid positions about 23 to about 60 and from amino acid positions about 82 to the end. In various embodiments, the hydrophobic segments are antigenic and targets for CG53135-specific antibodies.

In one embodiment, a CG53135 protein is a variant of FGF-20. It will be appreciated by those skilled in the art that DNA sequence polymorphisms that lead to changes in the amino acid sequences of the FGF-20 protein may exist within a population (e.g., the human population). Such genetic polymorphism in the FGF-20 gene may exist among individuals within a population due to natural allelic variation. Such natural allelic variations can typically result in 1-5% variance in the nucleotide sequence of the FGF-20 gene. Any and all such nucleotide variations and resulting amino acid polymorphisms in the FGF-20 protein, which are the result of natural allelic variation of the FGF-20 protein, are intended to be within the scope of the invention. Examples of FGF-20 SNPs can be found in Section 6, infra.

In another embodiment, CG53135 refers to a nucleic acid molecule encoding a FGF-20 protein from other species or the protein encoded thereby, and thus has a nucleotide or amino acid sequence that differs from the human sequence of FGF-20. Nucleic acid molecules corresponding to natural allelic variants and homologues of the FGF-20 cDNAs of the invention can be isolated based on their homology to the human FGF-20 nucleic acids disclosed herein using the human cDNAs, or a portion thereof, as a hybridization probe according to standard hybridization techniques under stringent hybridization conditions.

The invention also encompasses derivatives and analogs of FGF-20. The production and use of derivatives and analogs related to FGF-20 are within the scope of the present invention.

In a specific embodiment, the derivative or analog is functionally active, i.e., capable of exhibiting one or more functional activities associated with a full-length, wild-type FGF-20. Derivatives or analogs of FGF-20 can be tested for the desired activity by procedures known in the art, including but not limited to, using appropriate cell lines, animal models, and clinical trials.

In particular, FGF-20 derivatives can be made via altering FGF-20 sequences by substitutions, insertions or deletions that provide for functionally equivalent molecules. In one embodiment, such alteration of an FGF-20 sequence is done in a region that is not conserved in the FGF protein family. Due to the degeneracy of nucleotide coding sequences, other DNA sequences which encode substantially the same amino acid sequence as FGF-20 may be used in the practice of the present invention. These include, but are not limited to, nucleic acid sequences comprising all or portions of FGF-20 that are altered by the substitution of different codons that encode a functionally equivalent amino acid residue within the sequence, thus producing a silent change. In a preferred embodiment, a wild-type FGF-20 nucleic acid sequence is codon-optimized to the nucleic acid sequence of SEQ ID NO:8 (CG53135-05). Likewise, the FGF-20 derivatives of the invention include, but are not limited to, those containing, as a primary amino acid sequence, all or part of the amino acid sequence of FGF-20 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. FGF-20 derivatives of the invention also include, but are not limited to, those containing, as a primary amino acid sequence, all or part of the amino acid sequence of FGF-20 including altered sequences in which amino acid residues are substituted for residues with similar chemical properties. In a specific embodiment, 1, 2, 3, 4, or 5 amino acids are substituted.

Derivatives or analogs of FGF-20 include, but are not limited to, those proteins which are substantially homologous to FGF-20 or fragments thereof, or whose encoding nucleic acid is capable of hybridizing to the FGF-20 nucleic acid sequence.

The FGF-20 derivatives and analogs of the invention can be produced by various methods known in the art. The manipulations that result in their production can occur at the gene or protein level. For example, the cloned FGF-20 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of FGF-20, care should be taken to ensure that the modified gene remains within the same translational reading frame as FGF-20, uninterrupted by translational stop signals, in the gene region where the desired FGF-20 activity is encoded.

Additionally, the FGF-20-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB.RTM. linkers (Pharmacia), etc.

Manipulations of the FGF-20 sequence may also be made at the protein level. Included within the scope of the invention are FGF-20 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2-groups, free COOH-groups, OH-groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of FGF-20 can be chemically synthesized. For example, a protein corresponding to a portion of FGF-20 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the FGF-20 sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the FGF-20 derivative is a chimeric or fusion protein comprising FGF-20 or a fragment thereof fused via a peptide bond at its amino- and/or carboxy-terminus to a non-FGF-20 amino acid sequence. In one embodiment, the non-FGF-20 amino acid sequence is fused at the amino-terminus of an FGF-20 or a fragment thereof. In another embodiment, such a chimeric protein is produced by recombinant expression of a nucleic acid encoding the protein (comprising an FGF-20-coding sequence joined in-frame to a non-FGF-20 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding FGF-20 with a heterologous signal sequence is expressed such that the chimeric protein is expressed and processed by the cell to the mature FGF-20 protein. The primary sequence of FGF-20 and non-FGF-20 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of FGF-20 molecule fused to a heterologous (non-FGF-20) protein-encoding sequence may be constructed. In a specific embodiment, such chimeric construction can be used to enhance one or more desired properties of an FGF-20, including but not limited to, FGF-20 stability, solubility, or resistance to proteases. In another embodiment, chimeric construction can be used to target FGF-20 to a specific site. In yet another embodiment, chimeric construction can be used to identify or purify an FGF-20 of the invention, such as a His-tag, a FLAG tag, a green fluorescence protein (GFP), β-galactosidase, a maltose binding protein (MalE), a cellulose binding protein (CenA) or a mannose protein, etc. In one embodiment, a CG53135 protein is carbamylated.

In some embodiment, a CG53135 protein can be modified so that it has an extended half-life in vivo using any methods known in the art. For example, Fc fragment of human IgG or inert polymer molecules such as high molecular weight polyethyleneglycol (PEG) can be attached to a CG53135 protein. PEG can be attached to a CG53135 protein with or without a multifunctional linker either through site-specific conjugation of the PEG to the N— or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG53135 protein. Unreacted PEG can be separated from CG53135-PEG conjugates by size-exclusion or by ion-exchange chromatography. PEG-derivatized conjugates can be tested for in vivo efficacy using methods known to those of skill in the art.

A CG53135 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG53135 refers to CG53135-01 (SEQ ID NOs:1 and 2), CG53135-02 (SEQ ID NOs:3 and 4), CG53135-03 (SEQ ID NOs:5 and 2), CG53135-04 (SEQ ID NOs:6 and 7), CG53135-05 (SEQ ID NOs:8 and 2), CG53135-06 (SEQ ID NOs:9 and 10), CG53135-07 (SEQ ID NOs:11 and 12), CG53135-08 (SEQ ID NOs:13 and 14), CG53135-09 (SEQ ID NOs:15 and 16), CG53135-10 (SEQ ID NOs:17 and 18), CG53135-11 (SEQ ID NOs:19 and 20), CG53135-12 (SEQ ID NOs:21 and 22), CG53135-13 (SEQ ID NOs:23 and 24), CG53135-14 (SEQ ID NOs:25 and 26), CG53135-15 (SEQ ID NOs:27 and 28), CG53135-16 (SEQ ID NOs:29 and 30), CG53135-17 (SEQ ID NOs:31 and 32), IFC 250059629 (SEQ ID NOs:33 and 34), IFC 20059669 (SEQ ID NOs:35 and 36), IFC 317459553 (SEQ ID NOs:37 and 38), IFC 317459571 (SEQ ID NOs:39 and 40), IFC 250059596 (SEQ ID NOs:41 and 10), IFC316351224 (SEQ ID NOs:41 and 10), or a combination thereof. In a specific embodiment, a CG53135 is carbamylated, for example, a carbamylated CG53135-13 protein or a carbamylated CG53135-05 protein.

### 5.2. Methods of Preparing CG53135

Any techniques known in the art can be used in purifying a CG53135 protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG53135 may be monitored by one or more in vitro or in vivo assays. The purity of CG53135 can be assayed by any methods known in the art, such as but not limited to, gel electrophoresis. See Scopes, supra. In some embodiment, the CG53135 proteins employed in a composition of the invention can be in the range of 80 to 100 percent of the total mg protein, or at least 80%, at least 85%, at least 90%, at least 95%, or at least 98% of the total mg protein. In one embodiment, one or more CG53135 proteins employed in a composition of the invention is at least 99% of the total protein. In another embodiment, CG53135 is purified to apparent homogeneity, as assayed, e.g., by sodium dodecyl sulfate polyacrylamide gel electrophoresis.

Methods known in the art can be utilized to recombinantly produce CG53135 proteins. A nucleic acid sequence encoding a CG53135 protein can be inserted into an expression vector for propagation and expression in host cells.

An expression construct, as used herein, refers to a nucleic acid sequence encoding a CG53135 protein operably associated with one or more regulatory regions that enable expression of a CG53135 protein in an appropriate host cell. “Operably-associated” refers to an association in which the regulatory regions and the CG53135 sequence to be expressed are joined and positioned in such a way as to permit transcription, and ultimately, translation.

The regulatory regions necessary for transcription of CG53135 can be provided by the expression vector. A translation initiation codon (ATG) may also be provided if a CG53135 gene sequence lacking its cognate initiation codon is to be expressed. In a compatible host-construct system, cellular transcriptional factors, such as RNA polymerase, will bind to the regulatory regions on the expression construct to effect transcription of the modified CG53135 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG53135 gene sequence or to insert a CG53135 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG53135 sequence operably associated with regulatory regions can be directly introduced into appropriate host cells for expression and production of a CG53135 protein without further cloning. See, e.g., U.S. Pat. No. 5,580,859. The expression constructs can also contain DNA sequences that facilitate integration of a CG53135 sequence into the genome of the host cell, e.g., via homologous recombination. In this instance, it is not necessary to employ an expression vector comprising a replication origin suitable for appropriate host cells in order to propagate and express CG53135 in the host cells.

A variety of expression vectors may be used, including but are not limited to, plasmids, cosmids, phage, phagemids or modified viruses. Such host-expression systems represent vehicles by which the coding sequences of a CG53135 gene may be produced and subsequently purified, but also represent cells which may, when transformed or transfected with the appropriate nucleotide coding sequences, express CG53135 in situ. These include, but are not limited to, microorganisms such as bacteria (e.g., E. coli and B. subtilis) transformed with recombinant bacteriophage DNA, plasmid DNA or cosmid DNA expression vectors containing CG53135 coding sequences; yeast (e.g., Saccharomyces, Pichia) transformed with recombinant yeast expression vectors containing CG53135 coding sequences; insect cell systems infected with recombinant virus expression vectors (e.g., baculovirus) containing CG53135 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG53135 coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG53135 molecule. For example, mammalian cells such as Chinese hamster ovary cells (CHO) can be used with a vector bearing promoter element from major intermediate early gene of cytomegalocirus for effective expression of a CG53135 sequence (Foecking et al., 1986, Gene 45:101; and Cockeft et al., 1990, Bio/Technology 8:2).

In bacterial systems, a number of expression vectors may be advantageously selected depending upon the use intended for the CG53135 molecule being expressed. For example, when a large quantity of a CG53135 is to be produced, for the generation of pharmaceutical compositions of a CG53135 molecule, vectors that direct the expression of high levels of readily purified fusion protein products may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific proteases like enterokinase allows the CG53135 protein to be cleaved from the fusion protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG53135 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG53135 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG53135 in infected hosts (see, e.g., Logan & Shenk, 1984, Proc. Natl. Acad. Sci. USA 8 1:355-359). Specific initiation signals may also be required for efficient translation of inserted CG53135 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Biftner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells. Expression in a bacterial or yeast system can be used if post-translational modifications turn to be non-essential for a desired activity of CG53135. In a preferred embodiment, E. coli is used to express a CG53135 sequence.

For long-term, high-yield production of properly processed CG53135, stable expression in cells is preferred. Cell lines that stably express CG53135 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG53135 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11 (5):155-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Scd. USA 78:2072); and hygro, which confers resistance to hygromycin (Santerre et al., 1984, Gene 30:147). In addition, mutant cell lines including, but not limited to, tk-, hgprt- or aprt-cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG53135. Modified culture conditions and media may also be used to enhance production of CG53135. Any techniques known in the art may be applied to establish the optimal conditions for producing CG53135.

An alternative to producing CG53135 or a fragment thereof by recombinant techniques is peptide synthesis. For example, an entire CG53135, or a protein corresponding to a portion of CG53135, can be synthesized by use of a peptide synthesizer. Conventional peptide synthesis or other synthetic protocols well known in the art may be used.

Proteins having the amino acid sequence of CG53135 or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid-labile, and Fmoc, which is base-labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG53135 is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG53135 can be found in Section 6, infra.

### 5.3. Antibodies to CG53135

In various embodiments, monoclonal or polyclonal antibodies specific to CG53135, or a domain of CG53135, can be used in immunoassays to measure the amount of CG53135 or used in immunoaffinity purification of a CG53135 protein. A Hopp & Woods hydrophilic analysis (see Hopp & Woods, Proc. Natl. Acad. Sci. U.S.A. 78:3824-3828 (1981) can be used to identify hydrophilic regions of a protein, and to identify potential epitopes of a CG53135 protein. In a specific embodiment, CG53135-07, CG53135-08, CG53135-09, CG53135-10, or CG53135-11 protein is used to generate a CG53135-specific antibody.

The antibodies that immunospecifically bind to an CG53135 or an antigenic fragment thereof can be produced by any method known in the art for the synthesis of antibodies, in particular, by chemical synthesis or preferably, by recombinant expression techniques.

Polyclonal antibodies immunospecific for CG53135 or an antigenic fragment thereof can be produced by various procedures well-known in the art. For example, a CG53135 protein can be administered to various host animals including, but not limited to, rabbits, mice, and rats, to induce the production of sera containing polyclonal antibodies specific for the CG53135. Various adjuvants may be used to increase the immunological response, depending on the host species, including but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T Cell Hybridomas 563 681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene II or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1 134; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non human immuoglobulin. A humanized antibody comprises substantially all of at least one, and typically two, variable domains (Fab, Fab′, F(ab′)2, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibody can be produced using variety of techniques known in the art, including but not limited to, CDR grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489 498; Studnicka et al., 1994, Protein Engineering 7(6):805 814; and Roguska et al., 1994, PNAS 91:969 973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16):10678 84 (1997), Roguska et al., Protein Eng. 9(10):895 904 (1996), Couto et al., Cancer Res. 55 (23 Supp):5973s 5977s (1995), Couto et al., Cancer Res. 55(8):1717 22 (1995), Sandhu J S, Gene 150(2):409 10 (1994), and Pedersen et al., J. Mol. Biol. 235(3):959 73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323.)

Further, the antibodies that immunospecifically bind to CG53135 or an antigenic fragment thereof can, in turn, be utilized to generate anti-idiotype antibodies that “mimic” CG53135 or an antigenic peptide thereof using techniques well-known to those skilled in the art. (See, e.g., Greenspan & Bona, 1989, FASEB J. 7(5):437-444; and Nissinoff, 1991, J. Immunol. 147(8):2429-2438).

### 5.3.1 Polynucleotide Sequences Encoding an Antibody

The invention provides polynucleotides comprising a nucleotide sequence encoding an antibody or fragment thereof that immunospecifically binds to CG53135 or an antigenic fragment thereof. The invention also encompasses polynucleotides that hybridize under high stringency, intermediate, or lower stringency hybridization conditions to polynucleotides that encode an antibody of the invention.

The polynucleotides may be obtained, and the nucleotide sequence of the polynucleotides determined, by any method known in the art. The nucleotide sequence of antibodies immunospecific for a desired antigen can be obtained, e.g., from the literature or a database such as GenBank. Once the amino acid sequences of a CG53135 or an antigenic fragment thereof is known, nucleotide sequences encoding this antibody or a fragment thereof (e.g., a CDR) can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a particular antigen. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

### 5.3.2 Recombinant Expression of an Antibody

Recombinant expression of an antibody of the invention, derivative, analog or fragement thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well-known in the art. See, e.g., U.S. Pat. No. 6,331,415. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication No. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715, and those describe in Section 5.2., supra). The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 5.4. Structural Prediction and Functional Analysis of CG53135

Any methods known in the art can be used to determine the identity of a purified CG53135 protein of the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG53135 protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure.

The purity of a CG53135 protein of the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG53135 protein in a composition used in accordance to the instant invention is 80%-100% pure by densitometry, or at least 97%, at least 98%, or at least 99% pure by densitometry. In another preferred embodiment, a CG53135 protein in a composition used in accordance to the instant invention is more than 97%, more than 98%, or more than 99% pure by densitometry.

The biological activities and/or potency of CG53135 of the present invention can be determined by any methods known in the art. For example, compositions for use in therapy in accordance to the methods of the present invention can be tested in suitable cell lines for one or more activities that FGF-20 possesses (e.g., cellular proliferation stimulatory activity). Non-limiting examples of such assays are described in Section 6, infra.

Structure prediction, analysis of crystallographic data, sequence alignment, as well as homology modeling, can also be accomplished using computer software programs available in the art, such as BLAST, CHARMm release 21.2 for the Convex, and QUANTA v.3.3, (Molecular Simulations, Inc., York, United Kingdom). Other methods of structural analysis can also be employed. These include, but are not limited to, X-ray crystallography (Engstom, A., 1974, Biochem. Exp. Biol. 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.).

The half life of a protein is a measurement of protein stability and indicates the time necessary for a one half reduction in activity of the protein. The half-life of a CG53135 protein can be determined by any method measuring activity of CG53135 in samples from a subject over a period of time. The normalization to concentration of CG53135 in the sample can be done by, e.g., immunoassays using anti-CG53135 antibodies to measure the levels of the CG53135 molecules in samples taken over a period of time after administration of the CG53135, or detection of radiolabelled CG53135 molecules in samples taken from a subject after administration of the radiolabeled CG53135 molecules. In specific embodiments, techniques known in the art can be used to prolong the half life of an CG53135 in vivo. For example, albumin or inert polymer molecules such as high molecular weight polyethyleneglycol (PEG) can be used. See, e.g., International Publication Nos. WO 93/15199, WO 93/15200, and WO 01/77137; and U.S. Pat. No. 6,528,485.

Compositions comprising one more CG53135 for use in a therapy can also be tested in suitable animal model systems prior to testing in humans. To establish an estimate of drug activity in relevant model experiments, an index can be developed that combines observational examination of the animals as well as their survival status. The effectiveness of CG53135 in preventing and/or treating a disease can be monitored by any methods known to one skilled in the art, including but not limited to, dinical evaluation, and measuring the level of CG53135 biomarkers in a biosample.

Any adverse effects during the use of CG53135 alone or in combination with another therapy (e.g., another therapeutic or prophylactic agent) are preferably also monitored. Undesired effects typically experienced by patients taking one or more agents other than CG53135 are numerous and known in the art. Many are described in the Physicians' Desk Reference (58th ed., 2004).

### 5.5. Uses of CG53135

The present invention provides nucleic acids, proteins, and antibodies of CG53135, and their uses in preventing and/or treating a disorder associated with a pathology of epithelial cells and/or mesenchymal cells. In one embodiment, the present invention provides methods of preventing and/or treating a pathology of epithelial cells and/or mesenchymal cells comprising administering to a subject in need thereof a composition comprising one or more CG53135 proteins. In another embodiment, the present invention provides methods of stimulating proliferation, differentiation or migration of epithelial cells and/or mesenchymal cells comprising administering to a subject in need thereof an effective amount of a composition comprising one or more CG53135 proteins.

Epithelial membranes are continuous sheets of cells with contiguous cell borders that have characteristic specialized sites of close contact called cell junction. Such membrane, which can be one or more cells thick, contain no capillaries. Epithelia are attached to the underlying connective tissue by a component known as a basement membrane, which is a layer of intercellular material of complex composition that is distributed as a thin layer between the epithelium and the connective tissue.

Stratified squamous nonkeratinizing epithelium is common on wet surfaces that are subject to considerable wear and tear at sites where absorptive function is not required. The secretions necessary to keep such surfaces wet have to come from appropriately situated glands. Sites lined by this type of epithelium include the esophagus and the floor and sides of the oral cavity.

Simple columnar epithelium is made up of a single layer of tall cells that again fit together in a hexagonal pattern. In simple secretory columnar epithelium, the columnar cells are all specialized to secret mucus in addition to being protective. Sites of this type of epithelium is present include the lining of the stomach.

A simple columnar epithelium that is made up of absorptive cells as well as secretory cells lines the intestine. To facilitate absorption, this membrane is only one cell thick. Interspersed with cells that are specialized for absorption, there are many goblet cells that secrete protective mucus.

Mesenchymal cells are stem cells that can differentiate into, e.g., osteoblasts, chondrocytes, myocytes, and adipocytes. Mesenchymal-epithelial interactions play an important role in the physiology and pathology of epithelial tissues. Messenchymal cells may associate with epithelium basement membrane (e.g., pericytes and perivascular monocyte-derived cells (MDCs)), or reside within epithelium (MDCs and T cells). The nature of the interactions between mesenchymal cells and tissue-specific cells may depend on the tissue type (e.g., brain versus epidermis), or on the prevention or allowance/stimulation of differentiation of cells into the suicidal state (apoptosis) by mesenchymal cells in a given epithelium. Specialized mesenchymal cells, such as pericytes, MDCs, and T lymphocytes, may significantly influence the differentiation and aging of epithelial cells.

The stromal compartment of the cavities of bone is composed of a net-like structure of interconnected mesenchymal cells. Stromal cells are closely associated with bone cortex, bone trabecule and to the hemopoietic cells. The bone marrow-stromal micro-environment, is a complex of cells, extracellular matrix (ECM) with growth factors and cytokines that regulate osteogenesis and hemopoiesis locally throughout the life of the individual. The role of the marrow stroma in creating the microenvironment for bone physiology and hemopoiesis lies in a specific subpopulation of the stroma cells. They differentiate from a common stem cell to the specific lineage each of which has a different role. Their combined function results in orchestration of a 3-D-architecture that maintains the active bone marrow within the bone.

In adults, blood cells are produced by the bone marrow, the spongy material filling the body's bones. The bone marrow produces two blood cell groups, myeloid and lymphoid. The myeloid cell line includes, e.g., the following: (1) Immature cells called erythrocytes that later develop into red blood cells; (2) Blood clotting agents (platelets); (3) Some white blood cells, including macrophages (which act as scavengers for foreign particles), eosinophils (which trigger allergies and also defend against parasites), and neutrophils (the main defenders against bacterial infections). The lymphoid cell line includes, e.g., the lymphocytes, which are the body's primary infection fighters. Among other vital functions, certain lymphocytes are responsible for producing antibodies, factors that can target and attack specific foreign agents (antigens). Lymphocytes develop in the thymus gland or bone marrow and are therefore categorized as either B-cells (bone marrow-derived cells) or T-cells (thymus gland-derived cells).

According to the present invention, a CG53135 protein can regulate proliferation, differentiation, and/or migration of epithelial cells and/or mesenchymal cells, and thus have prophylactic and/or therapeutic effects on a disorder associated with a pathology of epithelial cells and/or mesenchymal cells.

Accordingly, CG53135 may also be used in wound and/or burn repairing and healing, ligament repairing, cartilage growth and/or repairing, promoting skin graft growth, increasing bone density, stimulating stem cell growth and/or differentiation, preventing and/or treating stroke, Alzheimer's disease, ischemic heart disease and/or aneurysms, or ulcers. Additional uses of CG53135 have been described in, e.g., U.S. patent application Ser. No. 10/435,087, filed May 9, 2003 (preventing and/or treating oral mucositis), Ser. No. 09/992,840, filed Nov. 6, 2001, Ser. No. 10/011,364, filed Nov. 16, 2001, and Ser. No. 10/321,962, filed Dec. 16, 2002 (preventing and/or treating inflammatory bowel disease (“IBD”)), Ser. No. 10/842,206, filed May 10, 2004 (preventing and/or treating arthritis and/or certain diseases related to central nerve system, such as Parkinson's Disease, and certain diseases related to cardiovascular system, such as stroke); and Ser. No. 10/842,179, filed May 10, 2004 (preventing and/or treating a disorder or symptom associated with radiation exposure). The content of each reference is incorporated herein by reference in its entirety.

Toxicity and therapeutic efficacy of a composition of the invention (e.g., a composition comprising one or more CG53135 proteins) can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀/ED₅₀. Compositions that exhibit large therapeutic indices are preferred. While compositions that exhibit toxic side effects may be used, care should be taken to design a delivery system that targets such composition to the site of affected tissue in order to minimize potential damage to uninfected cells and, thereby, reduce side effects.

In one embodiment, the data obtained from the cell culture assays and animal studies can be used in formulating a range of dosage for use in humans. The dosage of complexes lies preferably within a range of circulating concentrations that include the E₅₀ with little or no toxicity. The dosage may vary within this range depending upon the dosage form employed, the route of administration utilized, the severity of the disease, age and weight of the subject, and other factors normally considered by a medical professional (e.g., a physician). For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell cultures. Such information can be used to more accurately determine useful doses in humans. Levels in plasma may be measured, for example, by enzyme linked immunosorbent assays (ELISAs).

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more CG53135 proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 25 mg/kg, at least 50 mg/kg, at least 75 mg/kg, or at least 100 mg/kg (as measured by UV assay). In another embodiment, the dosage of a composition comprising one or more CG53135 proteins for administration in a human patient provided by the present invention is between 0.001-100 mg/kg, between 0.001-50 mg/kg, between 0.001-25 mg/kg, between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or by UV absorbance, and the concentration may vary depending on what assay is being used. In a non-limiting example, the protein concentration in a pharmaceutical composition of the instant invention is measured by UV absorbance that uses a direct measurement of the UV absorption at a wavelength of 280 nm, and calibration with a well characterized reference standard of CG53135 protein. Test results obtained with this UV method (using CG53135 reference standard) are three times lower than test results for the same sample(s) tested with the Bradford method. For example, if a dosage of a composition comprising one or more CG53135 proteins for administration in a human patient provided by the present invention is between 0.001-10 mg/kg measured by UV assay, then the dosage is 0.003-30 mg/kg as measured by Bradford assay.

The appropriate and recommended dosages, formulation and routes of administration for treatment modalities such as chemotherapeutic agents, radiation therapy and biological/immunotherapeutic agents such as cytokines, which can be used in combination with a composition comprising one or more CG53135, are known in the art and described in such literature as the Physician's Desk Reference (58th ed., 2004).

### 5.6. Administration, Pharmaceutical Compositions and Kits

Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG53135 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG53135 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG53135, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g.,arginine in various salt forms, sulfobutyl ether Beta-cyclodextrin sodium, or sucrose), excipient, or vehicle with which CG53135 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG53135 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG53135 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG53135 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG53135 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, fatty acids, fatty acid esters, steroids, chelating agents and surfactants. The compositions comprising CG53135 may be encapsulated within liposomes or may form complexes thereto, in particular to cationic liposomes. Alternatively, the compositions comprising CG53135 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG53135 can be formulated in an aerosol form, spray, mist or in the form of drops or powder if intranasal administration is preferred. In particular, a composition comprising CG53135 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG53135 proteins may also be formulated into a microcapsule with one or more polymers (e.g., hydroxyethyl starch) form the surface of the microcapsule. Such formulations have benefits such as slow-release.

A composition comprising CG53135 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG53135 can be delivered to a subject by pulmonary administration, e.g., by use of an inhaler or nebulizer, of a composition formulated with an aerosolizing agent.

In a preferred embodiment, a composition comprising CG53135 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG53135 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG53135 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

In some embodiments, a formulation used in accordance to the methods of the invention comprises 0.02 M-0.2 M acetate, 0.5-5% glycerol, 0.2-0.5 M arginine-HCl, and one ore more CG53135 proteins, preferably 0.05-5 mg/ml (UV). In one embodiment, a formulation used in accordance to the methods of the invention comprises 0.04M sodium acetate, 3% glycerol (volume/volume), 0.2 M arginine-HCl at pH 5.3, and one or more isolated CG53135 proteins, preferably 0.8 mg/ml (UV). In some embodiments, a formulation used in accordance to the methods of the invention comprises 0.01-1 M of a stabilizer, such as arginine in a salt form, sulfobutyl ether Beta-cyclodextrin sodium, or sucrose, 0.01-0.1 M sodium phosphate monobasic (NaH₂PO₄.H₂O), 0.01%-0.1% weight/volume (“w/v”) polysorbate 80 or polysorbate 20, and one or more CG53135 proteins, preferably 0.005-50 mg/ml (UV). In one embodiment, a formulation used in accordance to the methods of the invention comprises 30 mM sodium citrate, pH 6.1, 2 mM EDTA, 200 mM sorbitol, 50 mM KCl, 20% glycerol, and one or more isolated CG53135 proteins.

The invention also provides kits for carrying out the therapeutic regimens of the invention. Such kits comprise in one or more containers prophylactically or therapeutically effective amounts of the composition of the invention (e.g., a composition comprising one or more CG53135 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG53135 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### 6. EXAMPLES

The present invention is further illustrated by the following non-limiting examples.

### 6.1. Example 1

### Identification of the FGF-20 Gene

FGF-20 was identified following a TBLASTN (Altschul, et al. (1990) J. Mol. Biol. 215, 403-410) search of Genbank human genomic DNA sequences with Xenopus FGF-20 (Koga, et al. (1999) Biochem. Biophys. Res. Comm. 261, 756-765; Accession No. ABO12615) as query. This search identified a locus (Accession No. AB020858) of high homology on chromosome 8. Intron/exon boundaries were deduced using consensus splicing parameters (Mount (1996) Science 271, 1690-1692), together with homologies derived from known FGFs. The FGF-20 initiation codon localizes to base pair (“bp”) 16214 of the sequence of AB020858, and the remaining 3′ portion of this exon continues to bp 15930. The 5′ UTR of FGF-20 was extended upstream of the initiation codon by an additional 606 bp using public ESTs (Accession Nos. AA232729, AA236522, A1272876 and A1272878). The remaining structure of the FGF-20 gene as it relates to locus AB020858 is as follows: intron 1 (bp 15929-9942); exon 2 (bp 9941-9838); intron 2 (bp 9837-7500); exon 3 (begins at bp 7499 and continues as shown in Table 2; the structure of the 3′ untranslated region has not yet been determined). Table 2 presents an analysis of the FGF-20 gene, including the nucleotide and deduced amino acid (SEQ ID NO:2) sequence of FGF-20. The initiation and stop codons are in bold, and an in frame stop codon residing in the 5′ UTR is underlined.

TABLE 2

| | Exon 1 |
|---|---|
| | . . . AGACAGTGAGAGCTTCCCTGCCATTTCAGTGCAAAGTCCCTCCGGAGCGACCTCAGAGGAGTAACCGGGCCTTAACT |
| | TTTTGCGCTCGTTTTGCTATAATTTTTCTCTATCCACCTCCATCCCACCCCCACAACACTCTTTACTGGGGGGGTCTTTT |
| | GTGTTCCGGATCTCCCCCTCCATGGCTCCCTTAGCCGAAGTCGGGGGCTTTCTGGGCGGCCTGGAGGGCTTGGGCCAGCA |
| 1 | M A P L A E V G G F L G G L E G L G Q Q |
| | GGTGGGTTCGCATTTCCTGTTGCCTCCTGCCGGGGAGCGGCCGCCGCTGCTGGGCGAGCGCAGGAGCGCGGCGGAGCGGA |
| 21 | V G S H F L L P P A G E R P P L L G E R R S A A E R S |
| | GCGCGCGCGGCGGGCCGGGGGCTGCGCAGCTGGCGCACCTGCACGGCATCCTGCGCCGCCGGCAGCTCTATTGCCGCACC |
| 48 | A R G G P G A A Q L A H L H G I L R R R Q L Y C R T |
| | <- \| -> Exon 2 |
| | GGCTTCCACCTGCAGATCCTGCCCGACGGCAGCGTGCAGGGCACCCGGCAGGACCACAGCCTCTTCGGTATCTTGGAATT |
| 74 | G F H L Q I L P D G S V Q G T R Q D H S L F G I L E F |
| | CATCAGTGTGGCAGTGGGACTGGTCAGTATTAGAGGTGTGGACAGTGGTCTCTATCTTGGAATGAATGACAAAGGAGAAC |
| 101 | I S V A V G L V S I R G V D S G L Y L G M N D K G E L |
| | <- \| -> Exon 3 |
| | TCTATGGATCAGAGAAACTTACTTCCGAATGCATCTTTAGGGAGCAGTTTGAAGAGAACTGGTATAACACCTATTCATCT |
| 128 | Y G S E K L T S E C I F R E Q F E E N W Y N T Y S S |
| | AACATATATAAACATGGAGACACTGGCCGCAGGTATTTTGTGGCACTTAACAAAGACGGAACTCCAAGAGATGGCGCCAG |
| 154 | N I Y K H G D T G R R Y F V A L N K D G T P R D G A R |
| | GTCCAAGAGGCATCAGAAATTTACACATTTCTTACCTAGACCAGTGGATCCAGAAAGAGTTCCAGAATTGTACAAGGACC |
| 181 | S K R H Q K F T H F L P R P V D P E R V P E L Y K D L |
| | TACTGATGTACACTTGA . . . |
| 208 | L M Y T |

The gene discovered by the procedure in the preceding paragraph includes 3 exons and 2 introns (Table 2). The DNA sequence predicts an ORF of 211 amino acid residues, with an in-frame stop codon 117 bp upstream of the initiator methionine. The DNA segment from which the gene was mined maps to chromosome 8p21.3-p22, a location that was confirmed by radiation hybrid analysis (see Example 2).

An FGF signature motif, G-X-[LI]-X-[STAGP]-X(6,7)-[DE]-C-X-[FLM]-X-E-X(6)-Y, identified by a PROSITE search (Bucher & Bairoch (1994) Ismb. 2, 53-61) located between amino acid residues 125-148 is double-underlined, and intron/exon boundaries are depicted with arrows. Introns 1 and 2 are 5988 bp and 2338 bp long, respectively. The 5′ UTR sequence was derived from public ESTs, and is not shown in its entirety.

### 6.2. Example 2

### Radiation Hybrid Mapping of FGF-20

Radiation hybrid mapping using human chromosome markers was carried out for FGF-20. The procedure used is analogous to that described in Steen, R G et al. (A High-Density Integrated Genetic Linkage and Radiation Hybrid Map of the Laboratory Rat, Genome Research 1999 (Published Online on May 21, 1999)Vol. 9, AP1-AP8, 1999). A panel of 93 cell clones containing the randomized radiation-induced human chromosomal fragments was screened in 96 well plates using PCR primers designed to identify the sought clones in a unique fashion. The DNA segment from which the nucleotide sequence encoding FGF-20 was identified was annotated as mapping to chromosome 8p21.3-p22. This result was refined by the present analysis by finding that FGF-20 maps to chromosome 8 at a locus which overlaps marker AFM177XB10, and which is 1.6 cR from marker WI-5104 and 3.2 cR from marker WI-9262.

### 6.3. Example 3

### Molecular Cloning of the Sequence Encoding a FGF-20 Protein

Oligonucleotide primers were designed for the amplification by PCR of a DNA segment, representing an open reading frame, coding for the full length FGF-20. The forward primer includes a BglII restriction site (AGATCT) and a consensus Kozak sequence (CCACC). The reverse primer contains an in-frame XhoI restriction site for further subcloning purposes. Both the forward and the reverse primers contain a 5′ clamp sequence (CTCGTC). The sequences of the primers are the following:

FGF-20-Forward:

(SEQ ID NO: 42)

5′ - CTCGTC AGATCT CCACC ATG GCT CCC TTA GCC GAA
GTC -3′
FGF-20-Reverse:

(SEQ ID NO: 43)

5′ - CTCGTC CTCGAG AGT GTA CAT CAG TAG GTC CTT
G -3′

PCR reactions were performed using a total of 5 ng human prostate CDNA template, 1 μM of each of the FGF-20-Forward and FGF-20-Reverse primers, 5 micromoles dNTP (Clontech Laboratories, Palo Alto Calif.) and 1 microliter of 50×Advantage-HF 2 polymerase (Clontech Laboratories) in 50 microliter volume. The following PCR reaction conditions were used:
a) 96° C. 3 minutes
b) 96° C. 30 seconds denaturation
c) 70° C. 30 seconds, primer annealing. This temperature was gradually decreased by 1° C./cycle.
d) 72° C. 1 minute extension.
Repeat steps (b)-(d) ten times
e) 96° C. 30 seconds denaturation
f) 60° C. 30 secondsannealing
g) 72° C. 1 minute extension
Repeat steps (e)-(g) 25 times
h) 72° C. 5 minutes final extension

A single PCR product, with the expected size of approximately 640 bp, was isolated after electrophoresis on agarose gel and ligated into a pCR2.1 vector (Invitrogen, Carlsbad, Calif.). The cloned insert was sequenced using vector specific M13 Forward(-40) and M13 Reverse primers, which verified that the nucleotide sequence was 100% identical to the sequence in Table 1 (SEQ ID NO:1) inserted directly between the upstream BglII cloning site and the downstream XhoI cloning site. The cloned sequence constitutes an open reading frame coding for the predicted FGF-20 full length protein. The clone is called TA-AB02085-S274-F19.

### 6.4. Example 4

### Preparation of Mammalian ExDression Vector PCEP4/Sec

The oligonucleotide primers pSec-V5-His Forward (CTCGT CCTCG AGGGT AAGCC TATCC CTAAC) (SEQ ID NO:44) and pSec-V5-His Reverse (CTCGT CGGGC CCCTG ATCAG CGGGT TTAAA C) (SEQ ID NO: 45), were designed to amplify a fragment from the pcDNA3.1-V5His (Invitrogen, Carlsbad, Calif.) expression vector that includes V5 and His6. The PCR product was digested with XhoI and ApaI and ligated into the XhoI/ApaI digested pSecTag2 B vector harboring an Ig kappa leader sequence (Invitrogen, Carlsbad Calif.). The correct structure of the resulting vector, pSecV5His, including an in-frame Ig-kappa leader and V5-His6 was verified by DNA sequence analysis. The vector pSecV5His was digested with Pmel and Nhel to provide a fragment retaining the above elements in the correct frame. The Pmel-Nhel fragment was ligated into the BamHI/Klenow and Nhel treated vector pCEP4 (Invitrogen, Carlsbad, Calif.). The resulting vector was named pCEP4/Sec and includes an in-frame Ig kappa leader, a site for insertion of a clone of interest, and the V5 epitope and 6×His under control of the PCMV and/or the PT7 promoter. pCEP4/Sec is an expression vector that allows heterologous protein expression and secretion by fusing any protein into a multiple cloning site following the Ig kappa chain signal peptide. Detection and purification of the expressed protein are aided by the presence of the V5 epitope tag and 6×His tag at the C-terminus (Invitrogen, Carlsbad, Calif.).

### 6.5. Example 5

### Expression of FGF-20 in Human Embryonic Kidney (HEK) 293 Cells

The BglII-XhoI fragment containing the FGF-20 sequence was isolated from TA-AB02085-S274-F1 9 (Example 3) and subcloned into the BamHI-XhoI digested pCEP4/Sec to generate the expression vector pCEP4/Sec-FGF-20. The pCEP4/Sec-FGF-20 vector was transfected into 293 cells using the LipofectaminePlus reagent following the manufacturer's instructions (Gibco/BRL/Life Technologies, Rockville, Md.). The cell pellet and supernatant were harvested 72 hours after transfection and examined for FGF-20 expression by Western blotting (reducing conditions) with an anti-V5 antibody. FIG. 2 shows that FGF-20 is expressed as a polypeptide having an apparent molecular weight (Mr) of approximately 34 kDa proteins secreted by 293 cells. In addition a minor band is observed at about 31 kDa.

### 6.6. Example 6

### Expression of FGF-20 in E. coli

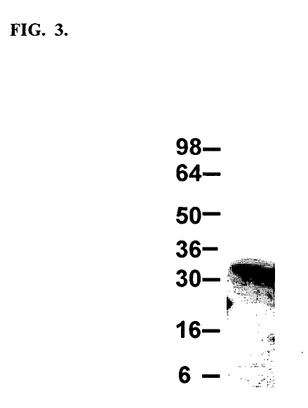

The vector PRSETA (InVitrogen Inc., Carlsbad, Calif.) was digested with XhoI and NcoI restriction enzymes. Oligonucleotide linkers of the sequence 5′ CATGGTCAGCCTAC 3′ (SEQ ID NO: 46) and 5′ TCGAGTAGGCTGAC 3′ (SEQ ID NO: 47) were annealed at 37 degree Celsius and ligated into the XhoI-NcoI treated pRSETA. The resulting vector was confirmed by restriction analysis and sequencing and was named pETMY. The BglII-XhoI fragment of the sequence encoding FGF-20 (see Example 3) was ligated into vector PETMY that was digested with BamHI and XhoIS restriction enzymes. The expression vector is named pETMY-FGF-20. In this vector, hFGF-20 was fused to the 6×His tag and T7 epitope at its N-terminus. The plasmid PETMY-FGF-20 was then transfected into the E. coli expression host BL21 (DE3, pLys) (Novagen, Madison, Wis.) and expression of protein FGF-20 was induced according to the manufacturer's instructions. After induction, total cells were harvested, and proteins were analyzed by Western blotting using anti-HisGly antibody (Invitrogen, Carlsbad, Calif.). FIG. 3 shows that FGF-20 was expressed as a protein of Mr approximately 32 kDa.

### 6.7. Example 7

### Comparison of Expression of Recombinant FGF-20 Protein With and Without a Cloned Signal Peptide.

### 6.7.1. Expression Without a Signal Peptide

As noted in the Detailed Description of the Invention, FGF-20 apparently lacks a classical amino-terminal signal sequence. To determine whether FGF-20 is secreted from mammalian cells, cDNA obtained as the BglII-XhoI fragment, encoding the full length FGF-20 protein, was subcloned from TA-AB02085-S274-F19 (Example 3) into BamHI/XhoI-digested pcDNA3.1 (Invitrogen). This provided a mammalian expression vector designated pFGF-20. This construct incorporates the V5 epitope tag and a polyhistidine tag into the carboxy-terminus of the protein to aid in its identification and purification, respectively, and should generate a polypeptide of about 27 kDa. Following transient transfection into 293 human embryonic kidney cells, conditioned media was harvested 48 hours post transfection.

In addition to secretion of tagged FGF-20 into conditioned media, it also found to be associated with the cell pellet/ECM. Since FGFs are known to bind to heparin sulfate proteoglycan (HSPG) present on the surface of cells and in the extracellular matrix (ECM), the inventors investigated the possibility that FGF-20 was sequestered in this manner. To this end, pFGF-20-transfected cells were extracted by treatment with 0.5 ml DMEM containing 100 μM suramin, a compound known to disrupt low affinity interactions between growth factors and HSPGs (La Rocca, R. V., Stein, C. A. & Myers, C. E. (1990) Cancer Cells 2, 106-115), for 30 min at 4° C. The suramin-extracted conditioned media was then harvested and clarified by centrifigation (5 min; 2000×g).

The conditioned media and the suramin extract were then mixed with equal volumes of 2× gel-loading buffer. Samples were boiled for 10 min, resolved by SDS-PAGE on 4-20% gradient polyacrylamide gels (Novex, Dan Diego, Calif.) under reducing conditions, and transferred to nitrocelluose filters (Novex). Western analysis was performed according to standard procedures using HRP-conjugated anti-V5 antibody (Invitrogen) and the ECL detection system (Amersham Pharmacia Biotech, Piscataway, N.J.).

One band having the expected molecular weight was identified in conditioned media from 293 cells transfected with pFGF-20 (FIG. 1A, lane 1). Conditioned media from cells transfected with control vector did not react with the antibody (FIG. 1A, lane 5). After suramin treatment, it was found that a significant quantity of tagged FGF-20 could in fact be released from the cell surface/ECM, indicating that HSPGs are likely to play a role in sequestering this protein (FIG. 1A, lane 2). These results indicate that FGF-20 can be secreted without a classical signal peptide.

Recombinant FGF-20 protein stimulates DNA synthesis and cell proliferation, effects that are likely to be mediated via high affinity binding of FGF-20 to a cell surface receptor, and modulated via low affinity interactions with HSPGs. The suramin extraction data suggests that FGF-20 binds to HSPGs present on the cell surface and/or the ECM.

### 6.7.2. Expression With a Signal Peptide

With the goal of enhancing protein secretion, a construct (pCEP4/Sec-FGF-20) was generated in which the FGF-20 cDNA was fused in frame with a cleavable amino-terminal secretory signal sequence derived from the IgK gene. The resulting protein also contained carboxy-terminal V5 and polyhistidine tags as described above for pFGF-20. Following transfection into 293 cells, a protein product having the expected molecular weight of about 31 kDa was obtained, and suramin was again found to release a significant quantity of sequestered FGF-20 protein (FIG. 1A; lanes 3 and 4). As expected, pCEP4/Sec-FGF-20 generated more soluble FGF-20 protein than did pFGF-20.

Results similar to those described above for 293 cells were also obtained with NIH 3T3 cells (FIG. 1B).

### 6.8. Example 8

### Real Time Quantitative Expression Analysis Of FGF-20 Nucleic Acids By PCR

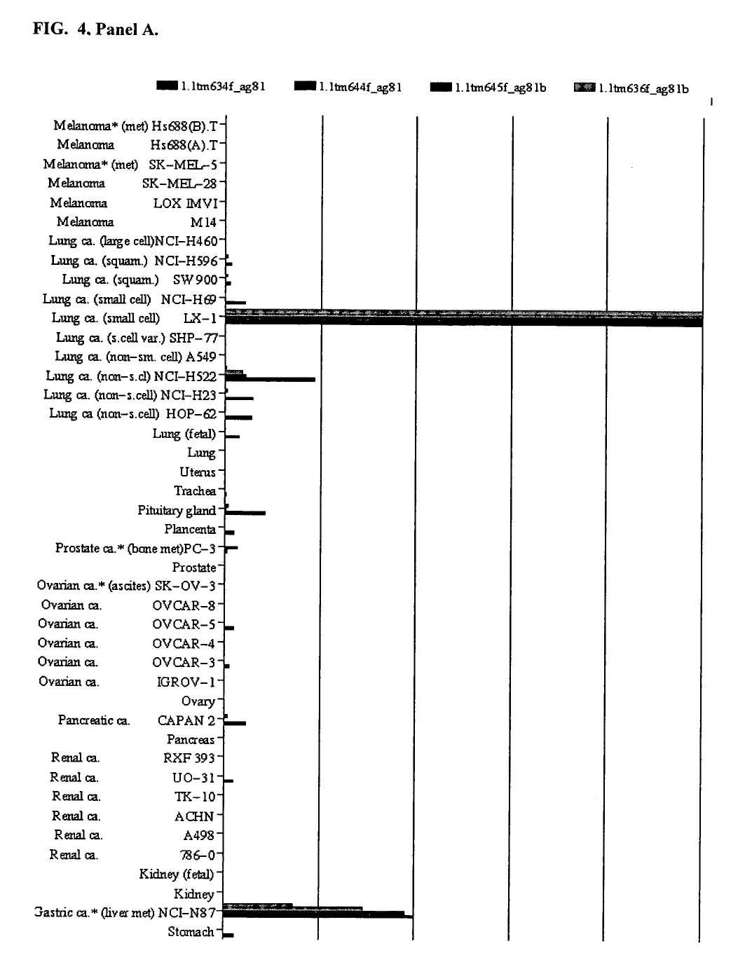

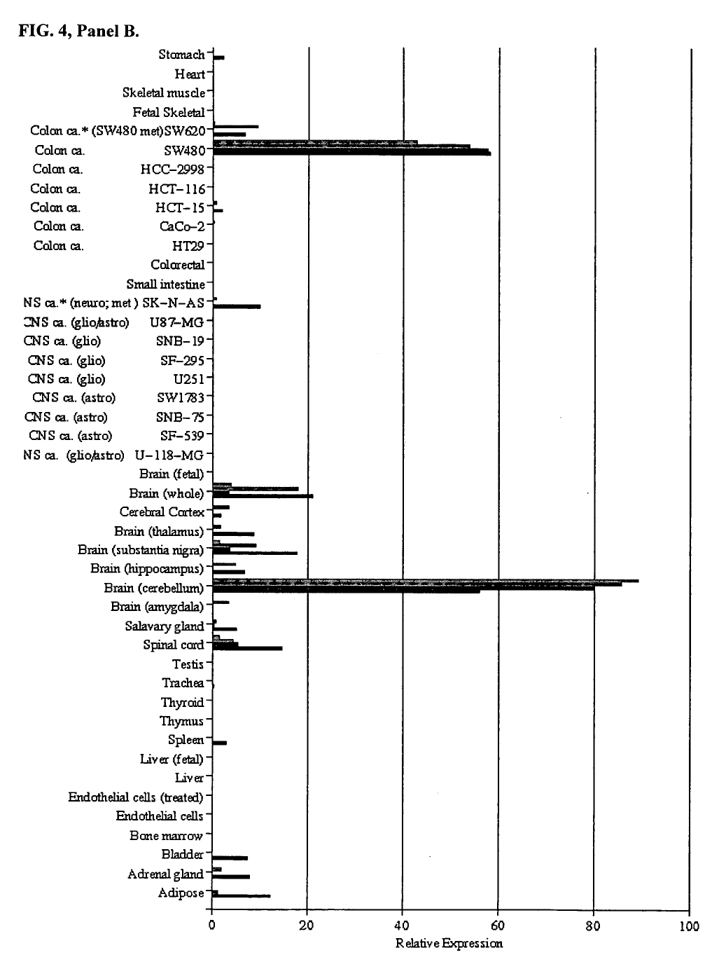

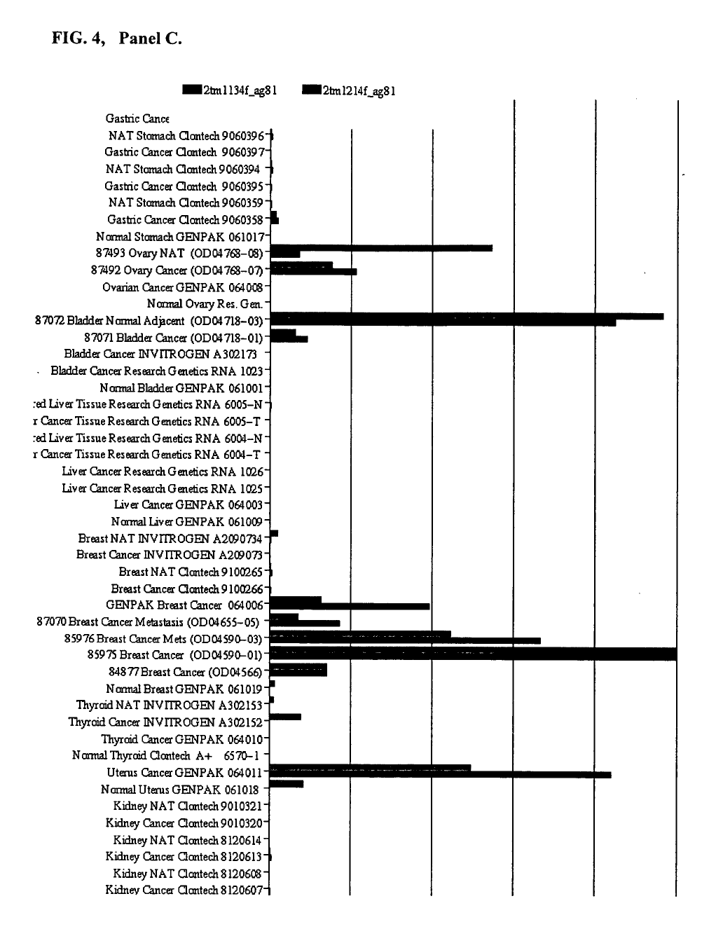

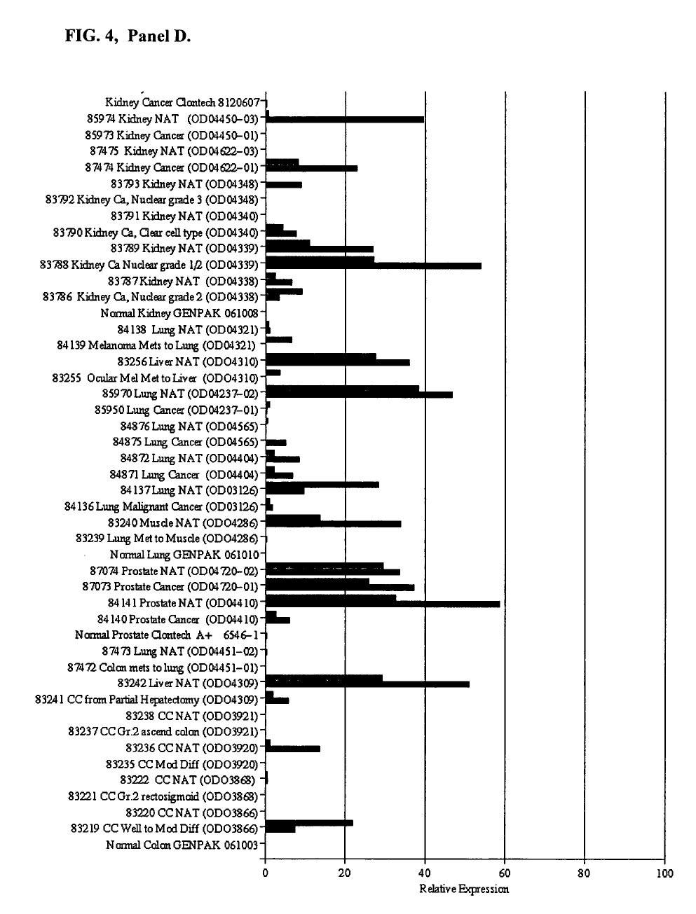

The quantitative expression of various clones was assessed in 41 normal and 55 tumor samples (in most cases, the samples presented in FIG. 4, Panels A and B are those identified in Table 3) by real time quantitative PCR (TAQMAN® analysis) performed on a Perkin-Elmer Biosystems ABI PRISM® 7700 Sequence Detection System. In Table 3, the following abbreviations are used:
ca.=carcinoma,
*=established from metastasis,
met=metastasis,
s cell var=small cell variant,
non-s=non-sm=non-small,
squam=squamous,
pi. eff=pi effusion=pleural effusion,
glio=glioma,
astro=astrocytoma, and
neuro=neuroblastoma.

First, 96 RNA samples were normalized to β-actin and glyceraldehyde-3-phosphate dehydrogenase (GAPDH). RNA (˜50 ng total or ˜1 ng polyA⁺) was converted to cDNA using the TAQMAN® Reverse Transcription Reagents Kit (PE Biosystems, Foster City, Calif.; cat # N808-0234) and random hexamers according to the manufacturer's protocol. Reactions were performed in 20 μl and incubated for 30 min. at 48° C. cDNA (5 μl) was then transferred to a separate plate for the TAQMAN® reaction using μ-actin and GAPDH TAQMAN® Assay Reagents (PE Biosystems; cat. no.'s 4310881 E and 4310884E, respectively) and TAQMAN® universal PCR Master Mix (PE Biosystems; cat # 4304447) according to the manufacturer's protocol. Reactions were performed in 25 μl using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 μl) was converted to cDNA and analyzed via TAQMAN® using One Step RT-PCR Master Mix Reagents (PE Biosystems; cat. #4309169) and gene-specific primers according to the manufacturer's instructions. Probes and primers were designed for each assay according to Perkin Elmer Biosystem's Primer Express Software package (version I for Apple Computer's Macintosh Power PC) using the sequence of clone 10326230.0.38 as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

For PCR, normalized RNA from each tissue and each cell line was spotted in each well of a 96 well PCR plate (Perkin Elmer Biosystems). PCR cocktails including two probes (one specific for FGF-20 and a second gene-specific probe to serve as an internal standard) were set up using 1× TaqMan™ PCR Master Mix for the PE Biosystems 7700, with 5 mM MgCl₂, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

TABLE 3
Tissue Samples used in TaqMan Expression Analysis

| No. | Tissue Sample |
|---|---|
| 1 | Endothelial cells |
| 2 | Endothelial cells (treated) |
| 3 | Pancreas |
| 4 | Pancreatic ca. CAPAN 2 |
| 5 | Adipose |
| 6 | Adrenal gland |
| 7 | Thyroid |
| 8 | Salivary gland |
| 9 | Pituitary gland |
| 10 | Brain (fetal) |
| 11 | Brain (whole) |
| 12 | Brain (amygdala) |
| 13 | Brain (cerebellum) |
| 14 | Brain (hippocampus) |
| 15 | Brain (hypothalamus) |
| 16 | Brain (Substantia nigra) |
| 17 | Brain (thalamus) |
| 18 | Spinal cord |
| 19 | CNS ca. (glio/astro) U87-MG |
| 20 | CNS ca. (glio/astro) U-118-MG |
| 21 | CNS ca. (astro) SW1783 |
| 22 | CNS ca.* (neuro; met) SK-N-AS |
| 23 | CNS ca. (astro) SF-539 |
| 24 | CNS ca. (astro) SNB-75 |
| 25 | CNS ca. (glio) SNB-19 |
| 26 | CNS ca. (glio) U251 |
| 27 | CNS ca. (glio) SF-295 |
| 28 | Heart |
| 29 | Skeletal muscle |
| 30 | Bone marrow |
| 31 | Thymus |
| 32 | Spleen |
| 33 | Lymph node |
| 34 | Colon (ascending) |
| 35 | Stomach |
| 36 | Small intestine |
| 37 | Colon ca. SW480 |
| 38 | Colon ca.* (SW480 met)SW620 |
| 39 | Colon ca. HT29 |
| 40 | Colon ca. HCT-116 |
| 41 | Colon ca. CaCo-2 |
| 42 | Colon ca. HCT-15 |
| 43 | Colon ca. HCC-2998 |
| 44 | Gastric ca.* (liver met) NCI-N87 |
| 45 | Bladder |
| 46 | Trachea |
| 47 | Kidney |
| 48 | Kidney (fetal) |
| 49 | Renal ca. 786-0 |
| 50 | Renal ca. A498 |
| 51 | Renal ca. RXF 393 |
| 52 | Renal ca. ACHN |
| 53 | Renal ca. UO-31 |
| 54 | Renal ca. TK-10 |
| 55 | Liver |
| 56 | Liver (fetal) |
| 57 | Liver ca. (hepatoblast) HepG2 |
| 58 | Lung |
| 59 | Lung (fetal) |
| 60 | Lung ca. (small cell) LX-1 |
| 61 | Lung ca. (small cell) NCI-H69 |
| 62 | Lung ca. (s. cell var.) SHP-77 |
| 63 | Lung ca. (large cell)NCI-H460 |
| 54 | Lung ca. (non-sm. cell) A549 |
| 55 | Lung ca. (non-s. cell) NCI-H23 |
| 66 | Lung ca (non-s. cell) HOP-62 |
| 57 | Lung ca. (non-s. cl) NCI-H522 |
| 68 | Lung ca. (squam.) SW 900 |
| 69 | Lung ca. (squam.) NCI-H596 |
| 70 | Mammary gland |
| 71 | Breast ca.* (pl. effusion) MCF-7 |
| 72 | Breast ca.* (pl. ef) MDA-MB-231 |
| 73 | Breast ca.* (pl. effusion) T47D |
| 74 | Breast ca. BT-549 |
| 75 | Breast ca. MDA-N |
| 76 | Ovary |
| 77 | Ovarian ca. OVCAR-3 |
| 78 | Ovarian ca. OVCAR-4 |
| 79 | Ovarian ca. OVCAR-5 |
| 80 | Ovarian ca. OVCAR-8 |
| 81 | Ovarian ca. IGROV-1 |
| 82 | Ovarian ca.* (ascites) SK-OV-3 |
| 83 | Myometrium |
| 84 | Uterus |
| 85 | Placenta |
| 86 | Prostate |
| 87 | Prostate ca.* (bone met)PC-3 |
| 88 | Testis |
| 89 | Melanoma Hs688(A).T |
| 90 | Melanoma* (met) Hs688(B).T |
| 91 | Melanoma UACC-62 |
| 92 | Melanoma M14 |
| 93 | Melanoma LOX IMVI |
| 94 | Melanoma* (met) SK-MEL-5 |
| 95 | Melanoma SK-MEL-28 |
| 96 | Melanoma UACC-257 |

The CG53135 gene disclosed in this invention is expressed in at least the following tissues: Mammalian Tissue, Colon, Lung, Brain, Liver, Kidney, and Stomach. Expression information was derived from the tissue sources of the sequences that were included in the derivation of the sequence of CG53135-02.

The following primers and probe were designed. Each possesses a minimum of three mismatches for corresponding regions of the highly homologous human FGF-9 and FGF-16 genes so as to be specific for FGF-20. Set Ag81b covers the region from base 270 to base 343 of Table 1 (SEQ ID NO:1). It should not detect other known FGF family members. The primers and probe utilized were:

Ag81b (F):

(SEQ ID NO: 48)

5′-GGACCACAGCCTCTTCGGTA-3′;
Ag81b (R):

(SEQ ID NO: 49)

5′-TGTCCACACCTCTAATACTGACCAG-3′;
and
Ag81b (P):

(SEQ ID NO: 50)

5′-FAM-CCCACTGCCACACTGATGAATTCCAA-TAMRA-3′.

The results from a representative experiment are shown in FIG. 4, Panels A and B. Expression is plotted as a percentage of the sample exhibiting the highest level of expression. Four replicate runs were made, presented in variously shaded bars. In 39 normal human tissues examined, CG53135 was found to be most highly expressed in the brain, particularly the cerebellum (FIG. 4, Panels A and B). Other tissues of the central nervous system expressed much lower levels of CG53135. Of the 54 human tumor cell lines examined, CG53135 was found to be most highly expressed in a lung carcinoma cell line (LX-1), a colon carcinoma cell line (SW-480) a colon cancer cell line and metastasis (SW480) and a gastric carcinoma cell line (NCI-N87; see FIG. 4, Panels A and B).

Additional real time expression analysis was done on an extensive panel of tumor tissues obtained during surgery. These tissues include portions obtained from the actual tumors themselves, as well as the portions termed “normal adjacent tissue (NAT)”, which typically are already inflamed and show histological evidence of dysplasia. A primer-probe set (Ag81) selected to be specific for CG53135 was employed in a TaqMan experiment with such surgical tissue samples, in which two replicate runs were performed:

Ag81 (F):

(SEQ ID NO: 51)

5′-AGGCAGAAGCGGGAGATAGAT-3′;
Ag81 (R):

(SEQ ID NO: 52)

5′-AGCAGCTTTACCTCATTCACAATG-3′;
and
Ag81 (P):

(SEQ ID NO: 53)

TET-5′-CCATCTACATCCACCACCAGTTGCAGAA-3′-TAMRA.

Set Ag81 covers the region from base 477 to base 554 of Table 1 (SEQ ID NO:1). The replicates are shown as bars of grey and black shading in FIG. 4, Panels C and D. The results show dramatically that for many matched pairs of tumors and their dysplastic NAT samples, CG53135 is highly expressed in the NAT but not in the tumor itself; more specifically, in the parenchymal cells adjacent to the tumor. Examples in which this matched pattern arises include ovarian cancer, bladder cancer, uterine cancer, lung cancer, prostate cancer and liver cancer.

### 6.9. Example 9

### Stimulation of Bromodeoxyuridine Incorporation by Recombinant CGS3135

293-EBNA cells (Invitrogen) were transfected using Lipofectamine 2000 according to the manufacturer's protocol (Life Technologies, Gaithersburg, Md.). Cells were supplemented with 10% fetal bovine serum (FBS; Life Technologies) 5 hours post-transfection. To generate protein for BrdU and growth assays (Example 10), cells were washed and fed with Dulbecco's modified Eagle medium (DMEM; Life Technologies) 18 hours post-transfection. After 48 hours, the media was discarded and the cell monolayer was incubated with 100 μM suramin (Sigma, St. Louis, Mo.) in 0.5 ml DMEM for 30 min at 4° C. The suramin-extracted conditioned media was then removed, clarified by centrifugation (5 min; 2000×g), and subjected to TALON metal affinity chromatography according to the manufacturer's instructions (Clontech, Palo Alto, Calif.) taking advantage of the carboxy-terminal polyhistidine tag. Retained fusion protein was released by washing the column with imidazole.

FGF-20 protein concentrations were estimated by Western analysis using a standard curve generated with a V5-tagged protein of known concentration. For Western analysis, conditioned media was harvested 48 hours post transfection, and the cell monolayer was then incubated with 0.5 ml DMEM containing 100 μM suramin for 30 min at 4° C. The suramin-containing conditioned media was then harvested.

To generate control protein, 293-EBNA cells were transfected with pCEP4 plasmid (Invitrogen) and subjected to the purification procedure outlined above.

Recombinant FGF-20 was tested for its ability to induce DNA synthesis in a bromodeoxyuridine (BrdU) incorporation assay. NIH 3T3 cells (ATCC number CRL-1658, American Type Culture Collection, Manassas, Va.), CCD-1070Sk cells (ATCC Number CRL-2091) or MG-63 cells (ATCC Number CRL-1427) were cultured in 96-well plates to ˜100% confluence, washed with DMEM, and serum-starved in DMEM for 24 hr (NIH 3T3) or 48 hours (CCD-1070Sk and MG-63). Recombinant FGF-20 or control protein was then added to the cells for 18 hours. The BrdU assay was performed according to the manufacturer's specifications (Roche Molecular Biochemicals, Indianapolis, Ind.) using a 5 hour BrdU incorporation time.

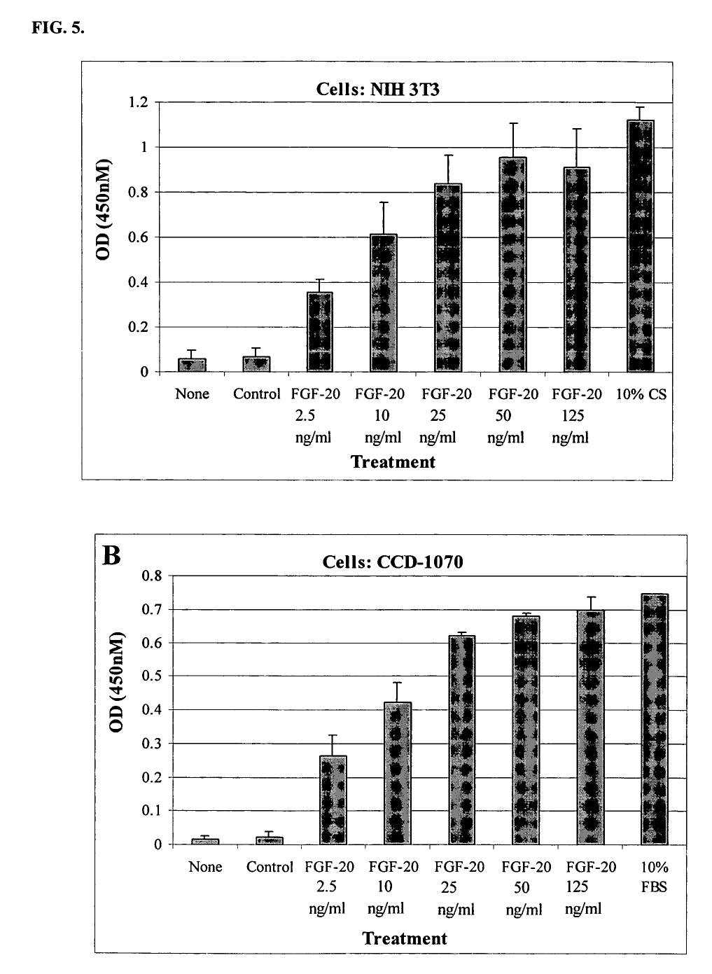

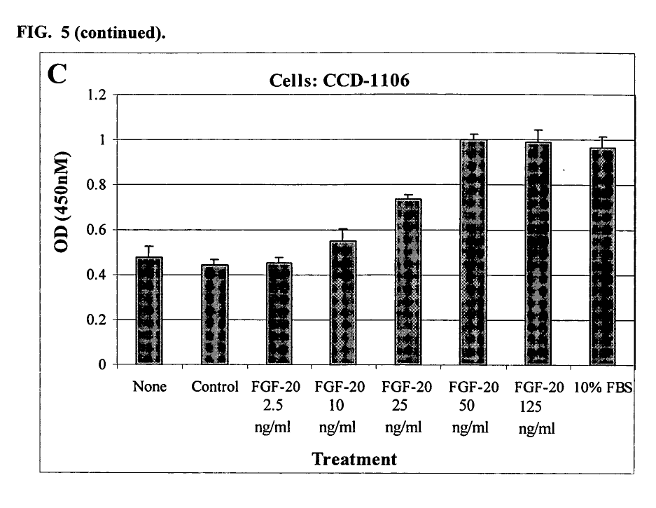

It was found that FGF-20 induced DNA synthesis in NIH 3T3 mouse fibroblasts at a half maximal concentration of ˜5 ng/ml (FIG. 5 Panel A). In contrast, protein purified from cells transfected with control vector did not induce DNA synthesis. It was also found that FGF-20 induces DNA synthesis, as determined by BrdU incorporation, at comparable dosing levels in a variety of human cell lines including CCD-1070Sk normal human skin fibroblasts (FIG. 5, Panel B), CCD-1106 keratinocytes (FIG. 5, Panel C), MG-63 osteosarcoma cells, and breast epithelial cells.

### 6.10. Example 10

### Expression of FGF-20

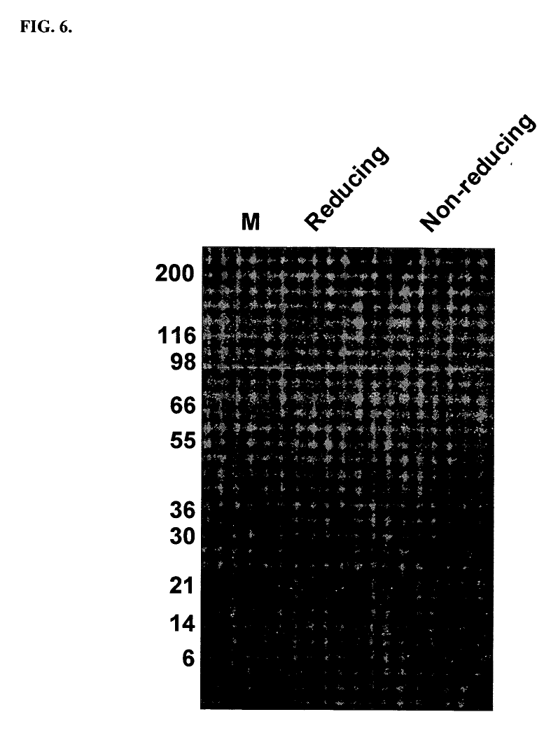

FGF-20 was expressed essentially as described in Example 6. The protein was purified using Ni²⁺-affinity chromatography, subjected to SDS-PAGE under both reducing and nonreducing conditions, and stained using Coomassie Blue. The results are shown in FIG. 6. It is seen that under both sets of conditions, the protein migrates with an apparent molecular weight of approximately 29-30 kDa.

### 6.11. Example 11

### Receptor Binding Specificity of FGF-20

Fibroblast growth factors (FGFs) play important roles in diverse functions including morphogenesis, cellular differentiation, angiogenesis, tissue remodeling, inflammation, and oncogenesis. FGFs contain a conserved 120-amino acid FGF core domain with a common tertiary structure. FGF signaling is generally assumed to occur by activation of transmembrane tyrosine kinase receptors. Four FGF receptors, FGFR1 through FGFR4, have been identified, and activating or inactivating receptor mutations have been described for a subset of these genes in both mice and humans.

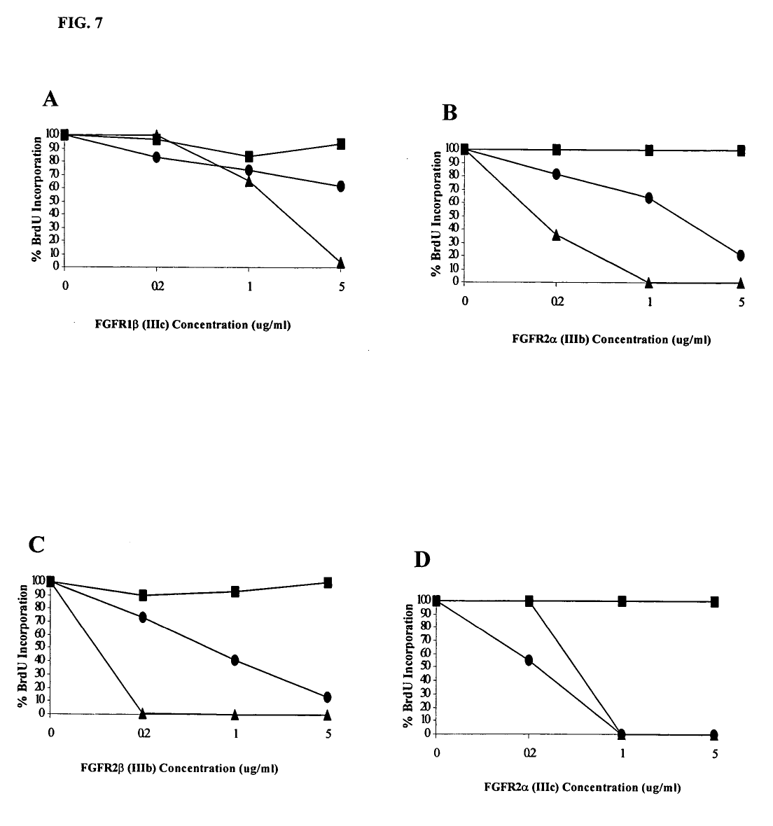

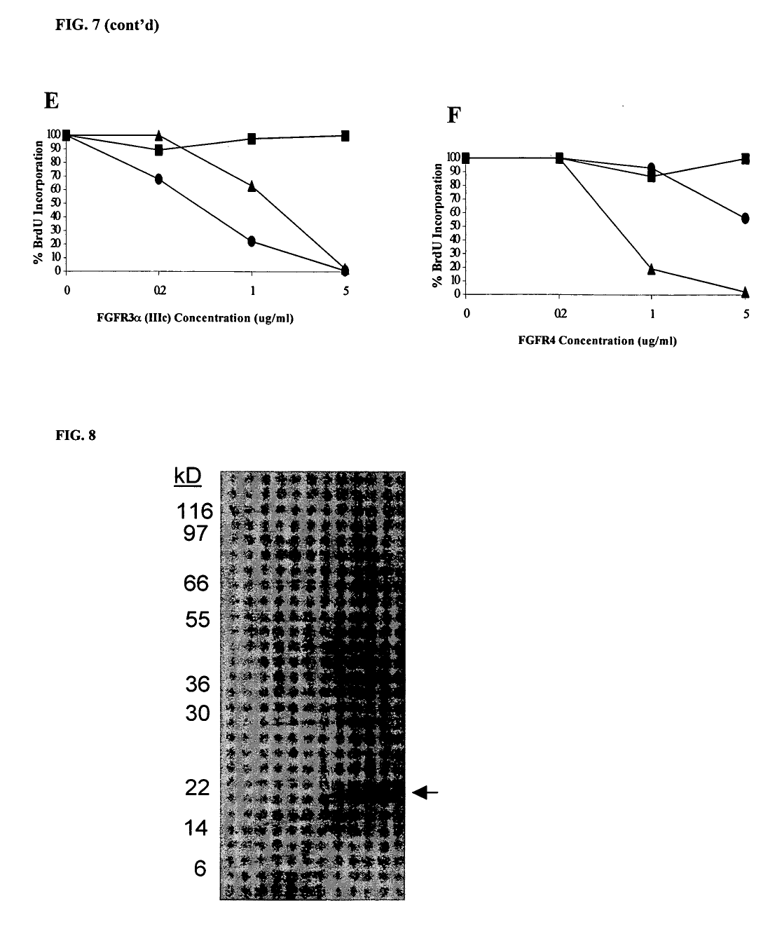

To determine the receptor binding specificity of FGF-20, we examined the effect of soluble FGF receptors (FGFRs) on the induction of DNA synthesis in NIH 3T3 cells by recombinant FGF-20. Four receptors have been identified to date (Klint P and Claesson-Welsh L. Front. Biosci., 4: 165-177, 1999; Xu X, et al. Cell Tissue Res., 296: 33-43, 1999). Soluble receptors for FGFR1β(IIIc), FGFR2α(IIIb), FGFR2β(IIIb), FGFR2α(IIIc), FGFR3α(IIIc) and FGFR4 were utilized. It was found that soluble forms of each of these FGFRs were able to specifically inhibit the biological activity of FGF-20 (see FIG. 7). Complete or nearly complete inhibition was obtained with soluble FGFR2α(IIIb), FGFR2β(IIIb), FGFR2α(IIIc), and FGFR3α(IIIc), whereas partial inhibition was achieved with soluble FGFR1β(IIIc) and FGFR4. None of the soluble receptor reagents interfered with the induction of DNA synthesis by PDGF-BB, thereby demonstrating their specificity. The integrity of each soluble receptor reagent was demonstrated by showing its ability to inhibit the induction of DNA synthesis by aFGF (acidic FGF), a factor known to interact with all of the FGFRs under analysis.

### 6.12. Example 12

### Cloning and Expression of an N-Terminal Deletion Form of FGF-20

E. coli strain BL21 (DE3) (Invitrogen) harboring the plasmid pET24a- FGF20X-del54-codon were grown in LB medium at 37° C. This plasmid encodes the C-terminal deletant of FGF-20 beginning at position 55. When cell densities reached an OD of 0.6, IPTG was added to final concentration of 1 mM. Induced cultures were then incubated for an additional 4 hours at 37° C. Cells were harvested by centrifugation at 3000×g for 15 minutes at 4° C., suspended in PBS and then disrupted with two passes through a microfluidizer. To separate soluble and insoluble proteins, the lysate was subjected to centrifugation at 10,000×g for 20 minutes at 4° C. The insoluble fraction (pellet) was extracted with PBS containing 1 M L-arginine. The remaining insoluble material was then removed by centrifugation and the soluble fraction of the arginine extract was filtered through 0.2 micron low-protein binding membrane and analyzed by SDS PAGE. The result is shown in FIG. 8, which indicates that the product is a polypeptide with an apparent molecular weight of approximately 20 kDa (see arrow). N-terminal sequencing of the expressed polypeptide provides the sequence AQLAHLHGILRRRQL which is 100% identical to residues 55-69 of FGF-20 (Table 1, SEQ ID NO:2).

### 6.13. Example 13

### Stimulation of Bromodeoxyuridine Incorporation into NIH 3T3 Cells in Response to a Truncated Form of FGF-20

A vector expressing residues 24-211 of FGF-20 ((d1-23)FGF-20; See Table 1 and SEQ ID NO:32 (CG53135-17) was prepared. The incorporation of BrdU by NIH 3T3 cells treated with conditioned medium obtained using the vector incorporating this truncated form was compared to the incorporation in response to treatment with conditioned medium using a vector encoding full length FGF-20. This experiment was carried out as described in Example 9.

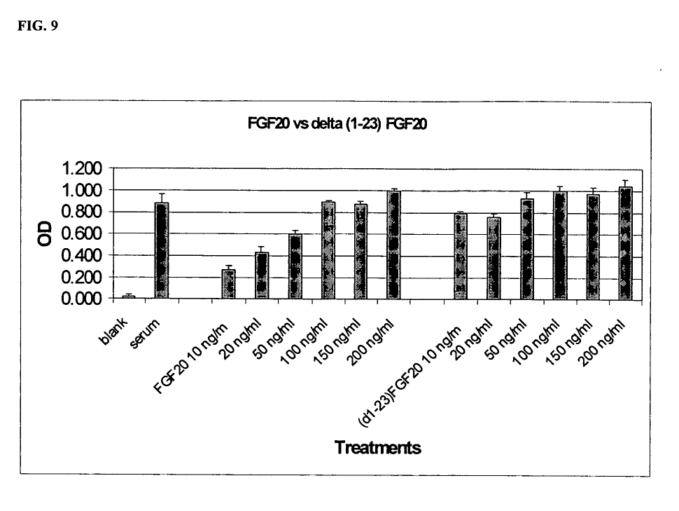

The results are shown in FIG. 9. It is seen that (d1-23)FGF-20 retains high activity at the lowest concentration tested, 10 ng/mL. At this concentration, the activity of full length FGF-20 has fallen considerably, approaching the level of the control. It is estimated that (d1-23)FGF-20 may be at least 5-fold more active than full length FGF-20.

### 6.14. Example 14

### Cloning and Expression of FGF-20 Variant CG53135-04

A nucleotide sequence encoding a variant of FGF-20, CG53135-04, was identified. The sequence of CG53135-04 was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof. The laboratory cloning was performed using one or more of the methods summarized below:

SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Exon Linking: The cDNA coding for the CG53135-04 sequence was cloned by the polymerase chain reaction (PCR) using the primers: 5′-AGGTCACCATGGCTGTTATTGGC-3′ (SEQ ID NO: 54) and 5′-CTGTCTGTCCTCAGAAGAAGTTCTTGATC-3′ (SEQ ID NO:55). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: The PCR product derived by exon linking, covering the entire open reading frame, was cloned into the pCR2.1 vector from Invitrogen to provide clone 137627::160083874.1043010.A9.

The DNA sequence and protein sequence for a novel Fibroblast Growth Factor-20-like gene were obtained by exon linking and are reported here as CG53135-04.

The novel nucleic acid of 540 nucleotides (CG53135-04) is shown in Table 4. An open reading frame was identified beginning at nucleotides 1-3 and ending at nucleotides 538-540. This polypeptide represents a novel functional Fibroblast Growth Factor-20-like protein. The start and stop codons of the open reading frame are highlighted in bold type. Putative untranslated regions (underlined), if any, are found upstream from the initiation codon and downstream from the termination codon. The encoded protein having 179 amino acid residues is presented using the one-letter code in Table 5.

TABLE 4
Nucleotide sequence of CG53135-04 (SEQ ID NO: 6)

| >CG53135-04 | |
|---|---|
| ATGGCTCCCTTAGCCGAAGTCGGGGGCTTTCTGGGCGGCCTGGAGGGCTTGGGCCAGCCG | 60 |
| GGGGCAGCGCAGCTGGCGCACCTGCACGGCATCCTGCGCCGCCGGCAGCTCTATTGCCGC | 120 |
| ACCGGCTTCCACCTGCAGATCCTGCCCGACGGCAGCGCGCAGGGCACCCGGCAGGACCAC | 180 |
| AGCCTCTTCGGTATCTTGGAATTCATCAGTGTGGCAGTGGGACTGGTCAGTATTAGAGGT | 240 |
| GTGGACAGTGGTCTCTATCTTGGAATGAATGACAAAGGAGAACTCTATGGATCAGAGAAA | 300 |
| CTTACTTCCGAATGCATCTTTAGGGAGCAGTTTGAAGAGAACTGGTATAACACCTATTCA | 360 |
| TCTAACATATATAAACATGGAGACACTGGCCGCAGGTATTTTGTGGCACTTAACAAAGAC | 420 |
| GGAACTCCAAGAGATGGCGCCAGGTCCAAGAGGCATCAGAAATTTACACATTTCTTACCT | 480 |
| AGACCAGTGGATCCAGAAAGAGTTCCAGAATTGTACAAGGACCTACTGATGTACACTTAG | 540 |

TABLE 5

Protein sequence encoded by the nucteotide
sequence shown in Table 4 above (SEQ ID NO: 7)

>CG53135-04
MAPLAEVGGFLGGLEGLGQPGAAQLAHLHGILRRRQLYCRTGFHLQILPD
GSAQGTRQDHSLFGILEFISVAVGLVSIRGVDSGLYLGMNDKGELYGSEK
LTSECIFREQFEENWYNTYSSNIYKHGDTGRRYFVALNKDGTPRDGARSK
RHQKFTHFLPRPVDPERVPELYKDLLMYT

The presence of identifiable domains in the protein disclosed herein was determined by searches versus domain databases such as Pfam, PROSITE, ProDom, Blocks or Prints and then identified by the Interpro domain accession number. Significant match was found to the IPR002209; (HBGF_FGF) domain, as summarized in Table 6.

TABLE 6
Domain Analysis for CG53135-04

| Model | Domain | seq-f | seq-t | hmm-f | hmm-t | score | E-value |
|---|---|---|---|---|---|---|---|
| FGF | 1/1 | 31 | 162 . . . | 1 | 146 [symbol] | 280.8 | 2e−81 |

IPR002209; (HBGF_FGF) Heparin-binding growth factors I and II (HBGF) (also known as acidic and basic fibroblast growth factors (FGF) are structurally related mitogens which stimulate growth or differentiation of a wide variety of cells of mesodermal or neuroectodermal origin. See, e.g., Burgess & Maciag, 1989) Annu. Rev. Biochem. 58: 575-606; Thomas 1988 Trends Biochem. Sci. 13: 327-328. These two proteins belong to a family of growth factors and oncogenes which is a member of a superfamily that also contains the interleukin-1 proteins, Kunitz-type soybean trypsin inhibitors (STI) and histactophilin. All have very similar structures, but although the HBGF and interleukin-1 families share some sequence similarity (about 25%), they show none at all to the STIs. See, e.g., Burgess & Maciag, 1989) Annu. Rev. Biochem. 58: 575-606; Thomas 1988 Trends Biochem. Sci. 13: 327-328; Heath et al. 1995 Curr. Biol. 5: 500-507; Matthews et al. 1991 Proc. Natl. Acad. Sci. U.S.A. 88: 3441-3445; Murzin 1992 J. Mol. Biol. 223: 531-543; Gimenez-Gallego et al. 1985 Science 230: 1385-1388; Copeland et al. 1996 Proc. Natl. Acad. Sci. U.S.A. 93: 9850-9857; and Ayres et al. 1994 Virology 202: 586-605.

HBGFs are involved in many different processes related to cell differentiation and growth control. See, e.g., Burgess & Maciag, 1989) Annu. Rev. Biochem. 58: 575-606. HBGF1 and HBGF2 have similar effects: they induce mesoderm formation in embryogenesis, and mediate wound repair, angiogenesis and neural outgrowth; they also induce proliferation and migration of fibroblasts, endothelial cells and astroglial cells. HBGF7, keratinocyte growth factor, is possibly the major paracrine effector of normal epithelial cell proliferation.

These growth factors cause dimerization of their tyrosine kinase receptors leading to intracellular signaling. There are currently four known tyrosine kinase receptors for fibroblast growth factors. These receptors can each bind several different members of this family. See, e.g., Heath et al. 1995 Curr. Biol. 5: 500-507.

The crystal structures of HBGF1 and HBGF2 have been solved. See, e.g., Matthews et al. 1991 Proc. Natl. Acad. Sci. U.S.A. 88: 3441-3445. HBGF1 and HBGF2 have the same twelve-stranded beta-sheet structure as both interleukin-1 and the Kunitz-type soybean trypsin inhibitors. See, e.g., Murzin 1992 J. Mol. Biol. 223: 531-543. HBGF1 and interleukin-1 had been found to be similar, and they were predicted to have similar structures. See, e.g., Gimenez-Gallego et al. 1985 Science 230: 1385-1388. The beta-sheets are arranged in three similar lobes around a central axis, six strands forming an anti-parallel beta-barrel. Several regions of HBGF1 have been implicated in receptor binding, notably beta-strands one through three, and the loop between strands eight and nine. The loop between strands ten and eleven is thought to be involved in binding heparin.

This indicates that the sequence of the invention has properties similar to those of other proteins known to contain the HBGF1-like and HBGF2-like domain(s) and similar to the properties of these domains.

The nucleic acids and proteins of the invention have applications in the diagnosis and/or treatment of various diseases and disorders. For example, the compositions of the present invention will have efficacy for the treatment of patients suffering from: Hirschsprung's disease, Crohn's Disease, appendicitis, inflammatory bowel disease, diverticular disease, systemic lupus erythematosus, autoimmune disease, asthma, emphysema, scleroderma, allergy, ARDS, Von Hippel-Lindau (VHL) syndrome, cirrhosis, transplantation, hypercalcemia, ulcers, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Alzheimer's disease, stroke, tuberous sclerosis, hypercalcemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration as well as other diseases, disorders and conditions.

### 6.15. Example 15

### Cloning and Expression of CG53135-06

A nucleotide sequence encoding a variant of FGF-20, referred to as CG53135-06, was identified, as shown in Tables 7 and 8. SeqCalling assembly sequences were initially identified by searching CuraGen Corporation's proprietary Human SeqCalling® database for DNA sequences that translate into proteins with similarity to SNP variant of FGF20 and/or members of the FGF20 family. One or more SeqCalling assemblies 174203299 were identified as having suitable similarity. The selected assembly was analyzed further to identify any open reading frames encoding novel full length proteins as well as novel splice forms. The resulting DNA sequence and protein sequence for a novel SNP variant of FGF20 gene are reported here as CG53135-06.

TABLE 7

Nucleotide sequence encoding CG53135-06
(SEQ ID NO: 9) of the invention

| | |
|---|---|
| >CG53135-06 | |
| ATGGCTCCCTTAGCCGAAGTCGGGGGCTTTCTGGGCGGCCTGGAGGGCTTGGGCCAGCCG | 60 |
| GGGGCAGCGCAGCTGGCGCACCTGCACGGCATCCTGCGCCGCCGGCAGCTCTATTGCCGC | 120 |
| ACCGGCTTCCACCTGCAGATCCTGCCCGACGGCAGCGTGCAGGGCACCCGGCAGGACCAC | 180 |
| AGCCTCTTCGGTATCTTGGAATTCATCAGTGTGGCAGTGGGACTGGTCAGTATTAGAGGT | 240 |
| GTGGACAGTGGTCTCTATCTTGGAATGAATGACAAAGGAGAACTCTATGGATCAGAGAAA | 300 |
| CTTACTTCCGAATGCATCTTTAGGGAGCAGTTTGAAGAGAACTGGTATAACACCTATTCA | 360 |
| TCTAACATATATAAACATGGAGACACTGGCCGCAGGTATTTTGTGGCACTTAACAAAGAC | 420 |
| GGAACTCCAAGAGATGGCGCCAGGTCCAAGAGGCATCAGAAATTTACACATTTCTTACCT | 480 |
| AGACCAGTGGATCCAGAAAGAGTTCCAGAATTGTACAAGGACCTACTGATGTACACTTAG | 540 |

TABLE 8

Protein sequence encoded by the nucleotide
sequence shown in Table 7 (SEQ ID NO: 10)

>CG53135-06
MAPLAEVGGFLGGLEGLGQPGAAQLAHLHGILRRRQLYCRTGFHLQILPD
GSVQGTRQDHSLFGILEFISVAVGLVSIRGVDSGLYLGMNDKGELYGSEK
LTSECIFREQFEENWYNTYSSNIYKHGDTGRRYFVALNKDGTPRDGARSK
RHQKFTHFLPRPVDPERVPELYKDLLMYT

A multiple sequence alignment is given in Table 9, with the protein of the invention being shown on the first line in a ClustalW analysis comparing the protein of the invention with related protein sequences. Note this sequence represents a SNP of CG53135-04 as indicated in position 53 of CG53135-06. Additional SNPs are described in Example 18, below.

[image]

The presence of identifiable domains in the protein disclosed herein was determined by searches versus domain databases such as Pfam, PROSITE, ProDom, Blocks or Prints and then identified by the Interpro domain accession number. Significant domains are summarized in Table 10. The IntroPro IPR002209 FGF domain is described above.

TABLE 10
Domain analysis for CG53135-06

| Model | Description | Score | E-value | N |
|---|---|---|---|---|
| FGF | (InterPro) Fibroblast growth factor | 286.8 | 3.4e−83 | 1 |

Parsed for domains:

| Model | Domain | seq-f | seq-t | hmm-f | hmm-t | score | E-value |
|---|---|---|---|---|---|---|---|
| FGF | 1/1 | 31 | 162 . . . | 1 | 146 [symbol] | 286.8 | 3.4e−83 |

### 6.16. Example 16

### Cloning and Characterization of FGF-20 Variants Including Optimized FGF-20

Additional FGF-20 variants were cloned as described above. Nucleotide and polypeptide are shown in Tables 11-17. Codon optimized FGF-20 is shown in Table 13.

TABLE 11

Nucleotide and Polypeptide Sequence for
CG53135-03, Consensus DNA Sequence:

>CG53135-03 636 nt
ATGGCTCCCTTAGCCGAAGTCGGGGGCTTTCTGGGCGGCCTGGAGGGCTT
GGGCCAGCAGGTGGGTTCGCATTTCCTGTTGCCTCCTGCCGGGGAGCGGC
CGCCGCTGCTGGGCGAGCGCAGGAGCGCGGCGGAGCGGAGCGCGCGCGGC
GGGCCGGGGGCTGCGCAGCTGGCGCACCTGCACGGCATCCTGCGCCGCCG
GCAGCTCTATTGCCGCACCGGCTTCCACCTGCAGATCCTGCCCGACGGCA
GCGTGCAGGGCACCCGGCAGGACCACAGCCTCTTCGGTATCTTGGAATTC
ATCAGTGTGGCAGTGGGACTGGTCAGTATTAGAGGTGTGGACAGTGGTCT
CTATCTTGGAATGAATGACAAAGGAGAACTCTATGGATCAGAGAAACTTA
CTTCCGAATGCATCTTTAGGGAGCAGTTTGAAGAGAACTGGTATAACACC
TATTCATCTAACATATATAAACATGGAGACACTGGCCGCAGGTATTTTGT
GGCACTTAACAAAGACGGAACTCCAAGAGATGGCGCCAGGTCCAAGAGGC
ATCAGAAATTTACACATTTCTTACCTAGACCAGTGGATCCAGAAAGAGTT
CCAGAATTGTACAAGGACCTACTGATGTACACTTGA
Protein Sequence:
ORF Start: 1 ORF Stop: 634 Frame: 1
>CG53135-03-prot 211 aa
MAPLAEVGGFLGGLEGLGQQVGSHFLLPPAGERPPLLGERRSAAERSARG
GPGAAQLAHLHGILRRRQLYCRTGFHLQILPDGSVQGTRQDHSLFGILEF
ISVAVGLVSIRGVDSGLYLGMNDKGELYGSEKLTSECIFREQFEENWYNT
YSSNIYKHGDTGRRYFVALNKDGTPRDGARSKRHQKFTHFLPRPVDPERV
PELYKDLLMYT

TABLE 12

Nucleotide and Polypeptide Sequence for
CG53135-04:Consensus DNA Sequence:

>CG53135-04 540 nt
ATGGCTCCCTTAGCCGAAGTCGGGGGCTTTCTGGGCGGCCTGGAGGGCTT
GGGCCAGCCGGGGGCAGCGCAGCTGGCGCACCTGCACGGCATCCTGCGCC
GCCGGCAGCTCTATTGCCGCACCGGCTTCCACCTGCAGATCCTGCCCGAC
GGCAGCGCGCAGGGCACCCGGCAGGACCACAGCCTCTTCGGTATCTTGGA
ATTCATCAGTGTGGCAGTGGGACTGGTCAGTATTAGAGGTGTGGACAGTG
GTCTCTATCTTGGAATGAATGACAAAGGAGAACTCTATGGATCAGAGAAA
CTTACTTCCGAATGCATCTTTAGGGAGCAGTTTGAAGAGAACTGGTATAA
CACCTATTCATCTAACATATATAAACATGGAGACACTGGCCGCAGGTATT
TTGTGGCACTTAACAAAGACGGAACTCCAAGAGATGGCGCCAGGTCCAAG
AGGCATCAGAAATTTACACATTTCTTACCTAGACCAGTGGATCCAGAAAG
AGTTCCAGAATTGTACAAGGACCTACTGATGTACACTTAG
>CG53135-04-prot 179 aa
MAPLAEVGGFLGGLEGLGQPGAAQLAHLHGILRRRQLYCRTGFHLQILPD
GSAQGTRQDHSLFGILEFISVAVGLVSIRGVDSGLYLGMNDKGELYGSEK
LTSECIFREQFEENWYNTYSSNIYKHGDTGRRYFVALNKDGTPRDGARSK
RHQKFTHFLPRPVDPERVPELYKDLLMYT

TABLE 13

Nucleotide and Polypeptide Sequence for Codon
Optimized FGF-20, CG53135-05

>CG53135-05 636 nt
ATGGCTCCGCTGGCTGAAGTTGGTGGTTTCCTGGGCGGTCTGGAGGGTCT
GGGTCAGCAGGTTGGTTCTCACTTCCTGCTGCCGCCGGCTGGTGAACGTC
CGCCACTGCTGGGTGAACGTCGCTCCGCAGCTGAACGCTCCGCTCGTGGT
GGCCCGGGTGCTGCTCAGCTGGCTCACCTGCATGGTATCCTGCGTCGCCG
TCAGCTGTACTGCCGTACTGGTTTCCACCTGCAGATCCTGCCGGATGGTT
CTGTTCAGGGTACCCGTCAGGACCACTCTCTGTTCGGTATCCTGGAATTC
ATCTCTGTTGCTGTTGGTCTGGTTTCTATCCGTGGTGTTGACTCTGGCCT
GTACCTGGGTATGAACGACAAAGGCGAACTGTACGGTTCTGAAAAACTGA
CCTCTGAATGCATCTTCCGTGAACAGTTTGAAGAGAACTGGTACAACACC
TACTCTTCCAACATCTACAAACATGGTGACACCGGCCGTCGCTACTTCGT
TGCTCTGAACAAAGACGGTACCCCGCGTGATGGTGCTCGTTCTAAACGTC
ACCAGAAATTCACCCACTTCCTGCCGCGCCCAGTTGACCCGGAGCGTGTT
CCAGAACTGTATAAAGACCTGCTGATGTACACCTAA
Protein Sequence:
>CG53135-05-prot 211 aa
MAPLAEVGGFLGGLEGLGQQVGSHFLLPPAGERPPLLGERRSAAERSARG
GPGAAQLAHLHGILRRRQLYCRTGFHLQILPDGSVQGTRQDHSLFGILEF
ISVAVGLVSIRGVDSGLYLGMNDKGELYGSEKLTSECIFREQFEENWYNT
YSSNIYKHGDTGRRYFVALNKDGTPRDGARSKRHQKFTHFLPRPVDPERV
PELYKDLLMYT

TABLE 14

Nucleotide and Polypeptide Sequence for
CG53135-07:

CG53135-07 54 nt
ATGGCTCCCTTAGCCGAAGTCGGGGGCTTTCTGGGCGGCCTGGAGGGCTT
GGGC
Protein Sequence of CG53135-07:
CG53135-07-prot 18 aa
MAPLAEVGGFLGGLEGLG

TABLE 15

Nucleotide and Polypeptide Sequence for
CG53135pep2, CG53135-08:

CG53135-08 63 nt
GAGCGGCCGCCGCTGCTGGGCGAGCGCAGGAGCGCGGCGGAGCGGAGCGC
GCGCGGCGGGCCG
CG53135-08-prot 21 aa
ERPPLLGERRSAAERSARGGP

TABLE 16

Nucleotide and Polypeptide Sequence for
CG53135pep3, CG53135-09:

CG53135-09 63 nt
CGCAGGTATTTTGTGGCACTTAACAAAGACGGAACTCCAAGAGATGGCGC
CAGGTCCAAGAGG
CG53135-09-prot 21 aa
RRYFVALNKDGTPRDGARSKR

TABLE 17

Nucleotide and Polypeptide Sequences for
CG53135pep4, CG53135-10:

CG53135-10 60 nt
CCTAGACCAGTGGATCCAGAAAGAGTTCCAGAATTGTACAAGGACCTACT
GATGTACACT
CG53135-10-prot 20 aa
PRPVDPERVPELYKDLLMYT

### 6.17. Example 17

### Cloning and Expression of FGF-20 SNP Variants

SeqCalling™ Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences identified in human genomic DNA are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nudeotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs were identified by analyzing genomic sequence assemblies generated by a process called Deep SNP Mining (DSM) in CuraGen's proprietary SNPTool algorithm. The SNPTool identifies variation in assemblies with the following criteria: SNPs are not analyzed within 10 base pairs on both ends of an alignment; window size (number of bases in a view) is 10; the allowed number of mismatches in a window is 2; minimum SNP base quality (PHRED score) is 23; minimum number of changes to score an SNP is 2/assembly position. SNPTool analyzes the assembly and displays SNP positions, associated individual variant sequences in the assembly, the depth of the assembly at that given position, the putative assembly allele frequency, SNP sequence variation, and the genomic DNA pool source. Sequence traces are then selected and brought into view for manual validation. Built-in FrameSearch software allows for the concurrent identification of amino acid changing SNPs. SNPs that border the intron/exon boundary were double checked by importing the SNP consensus into CuraTools and performing a 1×1 TBLASTN against the CGUID protein sequence of interest. Comprehensive SNP data analysis is then exported into the SNPCalling database.

Method of novel SNP Confirmation: SNPs are confirmed employing a validated method know as Pyrosequencing. Detailed protocols for Pyrosequencing can be found in: Alderborn et al. (2000). Genome Research. 10, Issue 8, August. 1249-1265. SNP results are shown in Table 18.

TABLE 18
Variants of nucleotide sequence described in FIG. 1

| | Nucleotides | | | Amino Acids | | |
|---|---|---|---|---|---|---|
| | Posi- | | Modi- | Posi- | | Modi- |
| Variant | tion | Initial | fied | tion | Initial | fied |
| 13377871 | 301 | A | G | 101 | Ile | Val |
| 13374151 | 308 | T | G | 103 | Val | Gly |
| 13375519 | 361 | A | G | 121 | Met | Val |
| 13375518 | 517 | G | A | 173 | Gly | Arg |
| 13375516 | 523 | C | G | 175 | Pro | Ala |
| 13375517 | 616 | G | A | 206 | Asp | Asn |

### 6.18. Example 18

### Molecular Cloning of FGF-20 Variant CG53135-06

### 6.18.1. Molecular Cloning of CG53135-06 Residue 1 to 179

The cDNA coding for the full-length form of CG53135-04 from residue 1 to 179 was targeted for “in-frame” cloning by PCR. The PCR template is based on the previously identified plasmid.

The following oligonucleotide primers were used to clone the target cDNA sequence:

F1

(SEQ ID NO: 56)

5′-CACCAGATCT ATGGCTCCCTTAGCCGAAGTCGGGGGC-3′
R1

(SEQ ID NO: 57)

5′-GCCGTCGAC AGTGTACATCAGTAGGTCCTTGTACAATTC-3′

For downstream cloning purposes, the forward primer includes an in-frame Bgl II restriction site and the reverse primer contains an in-frame Sal I restriction site.

Two PCR reactions were set up using a total of 1-5 ng of the plasmid that contains the insert for CG53135-06.

The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The following reaction conditions were used:
PCR condition 1:
a) 96° C. 3 minutes
b) 96° C. 30 seconds denaturation
c) 60° C. 30 seconds, primer annealing
d) 72° C. 6 minutes extension
Repeat steps b-d 15 times
e) 96° C. 15 seconds denaturation
f) 60° C. 30 seconds, primer annealing
g) 72° C. 6 minutes extension
Repeat steps e-g 29 times
e) 72° C. 10 minutes final extension
PCR condition 2:
a) 96° C. 3 minutes
b) 96° C. 15 seconds denaturation
c) 76° C. 30 seconds, reducing the temperature by 1° C. per cycle
d) 72° C. 4 minutes extension
Repeat steps b-d 34 times
e) 72° C. 10 minutes final extension.

An amplified product was detected by agarose gel electrophoresis. The fragment was gel-purified and ligated into the pCR2.1 TOPO vector (Invitrogen, Carlsbad, Calif.) following the manufacturer's recommendation. Twelve clones per PCR reaction were picked and sequenced. The inserts were sequenced using vector-specific M13 Forward and M13 Reverse primers.

| | | |
|---|---|---|
| SF1: | GTATCTTGGAATTCATCAGTGTGGC | (SEQ ID NO: 58) |
| SF2: | TGGTCTCTATCTTGGAATGAATGAC | (SEQ ID NO: 59) |
| SR1: | GAAGAGGCTGTGGTCCTGCC | (SEQ ID NO: 60) |
| SR2: | ACTGTCCACACCTCTAATACTGACC | (SEQ ID NO: 61) |

The insert assembly 250059596 was found to encode an open reading frame between residues 1 and 179 of the target sequence of CG53135-06. See Tables 19-22. The cloned inserts are 100% identical to the original sequence. The first 3 and the last 3 amino acid residues of the assemblies are derived from the restriction enzyme sites added in the primers for the purpose of sub-cloning. Note that differing amino acids have a white or grey background, and deleted/inserted amino acids can be detected by a dashed line in the sequence that does not code at that position.

TABLE 19
Cloned Sequences

>CG53135-06
ATGGCTCCCTTAGCCGAAGTCGGGGGCTTTCTGGGCGGCCTGGAGGGCTT
GGGCCAGCCGGGGGCAGCGCAGCTGGCGCACCTGCACGGCATCCTGCGCC
GCCGGCAGCTCTATTGCCGCACCGGCTTCCACCTGCAGATCCTGCCCGAC
GGCAGCGTGCAGGGCACCCGGCAGGACCACAGCCTCTTCGGTATCTTGGA
ATTCATCAGTGTGGCAGTGGGACTGGTCAGTATTAGAGGTGTGGACAGTG
GTCTCTATCTTGGAATGAATGACAAAGGAGAACTCTATGGATCAGAGAAA
CTTACTTCCGAATGCATCTTTAGGGAGCAGTTTGAAGAGAACTGGTATAA
CACCTATTCATCTAACATATATAAACATGGAGACACTGGCCGCAGGTATT
TTGTGGCACTTAACAAAGACGGAACTCCAAGAGATGGCGCCAGGTCCAAG
AGGCATCAGAAATTTACACATTTCTTACCTAGACCAGTGGATCCAGAAAG
AGTTCCAGAATTGTACAAGGACCTACTGATGTACACTTAG

TABLE 20
Cloned Sequences

>250059596
CACCAGATCTATGGCTCCCTTAGCCGAAGTCGGGGGCTTTCTGGGCGGCC
TGGAGGGCTTGGGCCAGCCGGGGGCAGCGCAGCTGGCGCACCTGCACGGC
ATCCTGCGCCGCCGGCAGCTCTATTGCCGCACCGGCTTCCACCTGCAGAT
CCTGCCCGACGGCAGCGTGCAGGGCACCCGGCAGGACCACAGCCTCTTCG
GTATCTTGGAATTCATCAGTGTGGCAGTGGGACTGGTCAGTATTAGAGGT
GTGGACAGTGGTCTCTATCTTGGAATGAATGACAAAGGAGAACTCTATGG
ATCAGAGAAACTTACTTCCGAATGCATCTTTAGGGAGCAGTTTGAAGAGA
ACTGGTATAACACCTATTCATCTAACATATATAAACATGGAGACACTGGC
CGCAGGTATTTTGTGGCACTTAACAAAGACGGAACTCCAAGAGATGGCGC
CAGGTCCAAGAGGCATCAGAAATTTACACATTTCTTACCTAGACCAGTGG
ATCCAGAAAGAGTTCCAGAATTGTACAAGGACCTACTGATGTACACTGTC
GACGGC

TABLE 21

View DNA Sequence Analysis of CG53135-06
Translated Protein - Frame: 1 - Nucleotide 1 to 537

| | |
|---|---|
| 1 | ATGGCTCCCTTAGCCGAAGTCGGGGGCTTTCTGGGCGGCCTGGAGGGCTTGGGCCAGCCGGGGGCAGCGCAGCTGGCGCA |
| | M A P L A E V G G F L G G L E G L G Q P G A A Q L A H |
| 81 | CCTGCACGGCATCCTGCGCCGCCGGCAGCTCTATTGCCGCACCGGCTTCCACCTGCAGATCCTGCCCGACGGCAGCGTGC |
| | L H G I L R R R Q L Y C R T G F H L Q I L P D G S V Q |
| 161 | AGGGCACCCGGCAGGACCACAGCCTCTTCGGTATCTTGGAATTCATCAGTGTGGCAGTGGGACTGGTCAGTATTAGAGGT |
| | G T R Q D H S L F G I L E F I S V A V G L V S I R G |
| 241 | GTGGACAGTGGTCTCTATCTTGGAATGAATGACAAAGGAGAACTCTATGGATCAGAGAAACTTACTTCCGAATGCATCTT |
| | V D S G L Y L G M N D K G E L Y G S E K L T S E C I F |
| 321 | TAGGGAGCAGTTTGAAGAGAACTGGTATAACACCTATTCATCTAACATATATAAACATGGAGACACTGGCCGCAGGTATT |
| | R E Q F E E N W Y N T Y S S N I Y K H G D T G R R Y F |
| 401 | TTGTGGCACTTAACAAAGACGGAACTCCAAGAGATGGCGCCAGGTCCAAGAGGCATCAGAAATTTACACATTTCTTACCT |
| | V A L N K D G T P R D G A R S K R H Q K F T H F L P |
| 481 | AGACCAGTGGATCCAGAAAGAGTTCCAGAATTGTACAAGGACCTACTGATGTACACTTAG |
| | R P V D P E R V P E L Y K D L L M Y T |

TABLE 22

View DNA Sequence Analysis of 250059596
Translated Protein - Frame: 2 - Nucleotide 2 to 556

| | |
|---|---|
| 1 | CACCAGATCTATGGCTCCCTTAGCCGAAGTCGGGGGCTTTCTGGGCGGCCTGGAGGGCTTGGGCCAGCCGGGGGCAGCGC |
| | T R S M A P L A E V G G F L G G L E G L G Q P G A A Q |
| 81 | AGCTGGCGCACCTGCACGGCATCCTGCGCCGCCGGCAGCTCTATTGCCGCACCGGCTTCCACCTGCAGATCCTGCCCGAC |
| | L A H L H G I L R R R Q L Y C R T G F H L Q I L P D |
| 161 | GGCAGCGTGCAGGGCACCCGGCAGGACCACAGCCTCTTCGGTATCTTGGAATTCATCAGTGTGGCAGTGGGACTGGTCAG |
| | G S V Q G T R Q D H S L F G I L E F I S V A V G L V S |
| 241 | TATTAGAGGTGTGGACAGTGGTCTCTATCTTGGAATGAATGACAAAGGAGAACTCTATGGATCAGAGAAACTTACTTCCG |
| | I R G V D S G L Y L G M N D K G E L Y G S E K L T S E |
| 321 | AATGCATCTTTAGGGAGCAGTTTGAAGAGAACTGGTATAACACCTATTCATCTAACATATATAAACATGGAGACACTGGC |
| | C I F R E Q F E E N W Y N T Y S S N I Y K H G D T G |
| 401 | CGCAGGTATTTTGTGGCACTTAACAAAGACGGAACTCCAAGAGATGGCGCCAGGTCCAAGAGGCATCAGAAATTTACACA |
| | R R Y F V A L N K D G T P R D G A R S K R H Q K F T H |
| 481 | TTTCTTACCTAGACCAGTGGATCCAGAAAGAGTTCCAGAATTGTACAAGGACCTACTGATGTACACTGTCGACGGC |
| | F L P R P V D P E R V P E L Y K D L L M Y T V D G |

### 6.18.2. Molecular Cloning of 31-162 amino acids of CG53135-06

The cDNA coding for the domain of CG53135-06 from residue 31 to 162 was targeted for “in-frame” cloning by PCR. The PCR template is based on the previously identified plasmid.

The following oligonucleotide primers were used to clone the target cDNA sequence:

| | | |
|---|---|---|
| F2 | 5′-CACCAGATCT ATCCTGCGCCGCCGGCA | (SEQ ID NO: 62) |
| | GCTCTATTGCC-3′ | |
| R2 | 5′-GCCGTCGAC TGGTCTAGGTAAGAAATG | (SEQ ID NO: 63) |
| | TGTAAATTTCTGATGCC-3′ | |

For downstream cloning purposes, the forward primer includes an in-frame Bgl II restriction site and the reverse primer contains an in-frame Sal I restriction site.

Two PCR reactions were set up using a total of 1-5 ng of the plasmid that contains the insert for CG53135-06.

The reaction mixtures contained 2 microliters of each of the primers (original concentration: 5 pmol/ul), 1 microliter of 10 mM dNTP (Clontech Laboratories, Palo Alto Calif.) and 1 microliter of 50× Advantage-HF 2 polymerase (Clontech Laboratories) in 50 microliter-reaction volume. The reaction conditions used are provided in above in Section 6.18.1.

An amplified product was detected by agarose gel electrophoresis. The fragment was gel-purified and ligated into the pCR2.1 TOPO vector (Invitrogen, Carlsbad, Calif.) following the manufacturer's recommendation. Twelve clones per PCR reaction were picked and sequenced. The inserts were sequenced using vector-specific M13 Forward and M13 Reverse primers.

The insert assembly 250059629 was found to encode an open reading frame between residues 31 and 162 of the target sequence of CG53135-06. The cloned inserts are 100% identical to the original sequence. See Tables 23-26. The alignment with CG53135-04 is displayed in a ClustalW in Table 27. The first 3 and the last 3 amino acid residues of the assemblies are derived from the restriction enzyme sites added in the primers for the purpose of sub-cloning. Note that differing amino acids have a white or grey background, and deleted/inserted amino acids can be detected by a dashed line in the sequence that does not code at that position.

TABLE 23
Cloned Sequences

>CG53135-06
ATGGCTCCCTTAGCCGAAGTCGGGGGCTTTCTGGGCGGCCTGGAGGGCTT
GGGCCAGCCGGGGGCAGCGCAGCTGGCGCACCTGCACGGCATCCTGCGCC
GCCGGCAGCTCTATTGCCGCACCGGCTTCCACCTGCAGATCCTGCCCGAC
GGCAGCGTGCAGGGCACCCGGCAGGACCACAGCCTCTTCGGTATCTTGGA
ATTCATCAGTGTGGCAGTGGGACTGGTCAGTATTAGAGGTGTGGACAGTG
GTCTCTATCTTGGAATGAATGACAAAGGAGAACTCTATGGATCAGAGAAA
CTTACTTCCGAATGCATCTTTAGGGAGCAGTTTGAAGAGAACTGGTATAA
CACCTATTCATCTAACATATATAAACATGGAGACACTGGCCGCAGGTATT
TTGTGGCACTTAACAAAGACGGAACTCCAAGAGATGGCGCCAGGTCCAAG
AGGCATCAGAAATTTACACATTTCTTACCTAGACCAGTGGATCCAGAAAG
AGTTCCAGAATTGTACAAGGACCTACTGATGTACACTTAG

TABLE 24
Cloned Sequences

>250059629
CACCAGATCTATCCTGCGCCGCCGGCAGCTCTATTGCCGCACCGGCTTCC
ACCTGCAGATCCTGCCCGACGGCAGCGTGCAGGGCACCCGGCAGGACCAC
AGCCTCTTCGGTATCTTGGAATTCATCAGTGTGGCAGTGGGACTGGTCAG
TATTAGAGGTGTGGACAGTGGTCTCTATCTTGGAATGAATGACAAAGGAG
AACTCTATGGATCAGAGAAACTTACTTCCGAATGCATCTTTAGGGAGCAG
TTTGAAGAGAACTGGTATAACACCTATTCATCTAACATATATAAACATGG
AGACACTGGCCGCAGGTATTTTGTGGCACTTAACAAAGACGGAACTCCAA
GAGATGGCGCCAGGTCCAAGAGGCATCAGAAATTTACACATTTCTTACCT
AGACCAGTCGACGGC

TABLE 25

View DNA Sequence Analysis of CG53135-06
Translated Protein - Frame: 1 - Nucleotide 1 to 537

| | |
|---|---|
| 1 | ATGGCTCCCTTAGCCGAAGTCGGGGGCTTTCTGGGCGGCCTGGAGGGCTTGGGCCAGCCGGGGGCAGCGCAGCTGGCGCA |
| | M A P L A E V G G F L G G L E G L G Q P G A A Q L A H |
| 81 | CCTGCACGGCATCCTGCGCCGCCGGCAGCTCTATTGCCGCACCGGCTTCCACCTGCAGATCCTGCCCGACGGCAGCGTGC |
| | L H G I L R R R Q L Y C R T G F H L Q I L P D G S V Q |
| 161 | AGGGCACCCGGCAGGACCACAGCCTCTTCGGTATCTTGGAATTCATCAGTGTGGCAGTGGGACTGGTCAGTATTAGAGGT |
| | G T R Q D H S L F G I L E F I S V A V G L V S I R G |
| 241 | GTGGACAGTGGTCTCTATCTTGGAATGAATGACAAAGGAGAACTCTATGGATCAGAGAAACTTACTTCCGAATGCATCTT |
| | V D S G L Y L G M N D K G E L Y G S E K L T S E C I F |
| 321 | TAGGGAGCAGTTTGAAGAGAACTGGTATAACACCTATTCATCTAACATATATAAACATGGAGACACTGGCCGCAGGTATT |
| | R E Q F E E N W Y N T Y S S N I Y K H G D T G R R Y F |
| 401 | TTGTGGCACTTAACAAAGACGGAACTCCAAGAGATGGCGCCAGGTCCAAGAGGCATCAGAAATTTACACATTTCTTACCT |
| | V A L N K D G T P R D G A R S K R H Q K F T H F L P |
| 481 | AGACCAGTGGATCCAGAAAGAGTTCCAGAATTGTACAAGGACCTACTGATGTACACTTAG |
| | R P V D P E R V P E L Y K D L L M Y T |

TABLE 26

View DNA Sequence Analysis of 250059629
Translated Protein - Frame: 2 - Nucleotide 2 to 415

| | |
|---|---|
| 1 | CACCAGATCTATCCTGCGCCGCCGGCAGCTCTATTGCCGCACCGGCTTCCACCTGCAGATCCTGCCCGACGGCAGCGTGC |
| | T R S I L R R R Q L Y C R T G F H L Q I L P D G S V Q |
| 81 | AGGGCACCCGGCAGGACCACAGCCTCTTCGGTATCTTGGAATTCATCAGTGTGGCAGTGGGACTGGTCAGTATTAGAGGT |
| | G T R Q D H S L F G I L E F I S V A V G L V S I R G |
| 161 | GTGGACAGTGGTCTCTATCTTGGAATGAATGACAAAGGAGAACTCTATGGATCAGAGAAACTTACTTCCGAATGCATCTT |
| | V D S G L Y L G M N D K G E L Y G S E K L T S E C I F |
| 241 | TAGGGAGCAGTTTGAAGAGAACTGGTATAACACCTATTCATCTAACATATATAAACATGGAGACACTGGCCGCAGGTATT |
| | R E Q F E E N W Y N T Y S S N I Y K H G D T G R R Y F |
| 321 | TTGTGGCACTTAACAAAGACGGAACTCCAAGAGATGGCGCCAGGTCCAAGAGGCATCAGAAATTTACACATTTCTTACCT |
| | V A L N K D G T P R D G A R S K R H Q K F T H F L P |
| 401 | AGACCAGTCGACGGC |
| | R P V D G |

[image]

The cDNA coding for the mature form of CG53135-06 from residue 31 to 179 was targeted for “in-frame” cloning by PCR. The PCR template is based on the previously identified plasmid.

The following oligonucleotide primers were used to clone the target cDNA sequence:

F2

(SEQ ID NO: 64)

5′-CACCAGATCT ATCCTGCGCCGCCGGCAGCTCTATTGCC-3′
R1

(SEQ ID NO: 65)

5′-GCCGTCGAC AGTGTACATCAGTAGGTCCTTGTACAATTC-3′

For downstream cloning purposes, the forward primer includes an in-frame Bgl II restriction site and the reverse primer contains an in-frame Sal I restriction site. Two PCR reactions were set up using a total of 1-5 ng of the plasmid that contains the insert for CG53135-06. The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The reaction conditions used are provided in above in Section 6.18.1.

An amplified product was detected by agarose gel electrophoresis. The fragment was gel-purified and ligated into the pCR2.1 TOPO vector (Invitrogen, Carlsbad, Calif.) following the manufacturer's recommendation. Twelve clones per PCR reaction were picked and sequenced. The inserts were sequenced using vector-specific M13 Forward and M13 Reverse primers.

The insert assembly 250059669 was found to encode an open reading frame between residues 31 and 179 of the target sequence of CG53135-06. The cloned inserts are 100% identical to the original sequence. See Tables 28-31. The alignment with CG53135-06 is displayed in a ClustalW in Table 32. The first 3 and the last 3 amino acid residues of the assemblies are derived from the restriction enzyme sites added in the primers for the purpose of sub-cloning. Note that differing amino acids have a white or grey background, and deleted/inserted amino acids can be detected by a dashed line in the sequence that does not code at that position.

TABLE 28
Cloned Sequences

>CG53135-06
ATGGCTCCCTTAGCCGAAGTCGGGGGCTTTCTGGGCGGCCTGGAGGGCTT
GGGCCAGCCGGGGGCAGCGCAGCTGGCGCACCTGCACGGCATCCTGCGCC
GCCGGCAGCTCTATTGCCGCACCGGCTTCCACCTGCAGATCCTGCCCGAC
GGCAGCGTGCAGGGCACCCGGCAGGACCACAGCCTCTTCGGTATCTTGGA
ATTCATCAGTGTGGCAGTGGGACTGGTCAGTATTAGAGGTGTGGACAGTG
GTCTCTATCTTGGAATGAATGACAAAGGAGAACTCTATGGATCAGAGAAA
CTTACTTCCGAATGCATCTTTAGGGAGCAGTTTGAAGAGAACTGGTATAA
CACCTATTCATCTAACATATATAAACATGGAGACACTGGCCGCAGGTATT
TTGTGGCACTTAACAAAGACGGAACTCCAAGAGATGGCGCCAGGTCCAAG
AGGCATCAGAAATTTACACATTTCTTACCTAGACCAGTGGATCCAGAAAG
AGTTCCAGAATTGTACAAGGACCTACTGATGTACACTTAG

TABLE 29
Cloned Sequences

>250059669
CACCAGATCTATCCTGCGCCGCCGGCAGCTCTATTGCCGCACCGGCTTCC
ACCTGCAGATCCTGCCCGACGGCAGCGTGCAGGGCACCCGGCAGGACCAC
AGCCTCTTCGGTATCTTGGAATTCATCAGTGTGGCAGTGGGACTGGTCAG
TATTAGAGGTGTGGACAGTGGTCTCTATCTTGGAATGAATGACAAAGGAG
AACTCTATGGATCAGAGAAACTTACTTCCGAATGCATCTTTAGGGAGCAG
TTTGAAGAGAACTGGTATAACACCTATTCATCTAACATATATAAACATGG
AGACACTGGCCGCAGGTATTTTGTGGCACTTAACAAAGACGGAACTCCAA
GAGATGGCGCCAGGTCCAAGAGGCATCAGAAATTTACACATTTCTTACCT
AGACCAGTGGATCCAGAAAGAGTTCCAGAATTGTACAAGGACCTACTGAT
GTACACTGTCGACGGC

TABLE 30

View DNA Sequence Analysis of CG53135-06
Translated Protein - Frame: 1 - Nucleotide 1 to 537

| | |
|---|---|
| 1 | ATGGCTCCCTTAGCCGAAGTCGGGGGCTTTCTGGGCGGCCTGGAGGGCTTGGGCCAGCCGGGGGCAGCGCAGCTGGCGCA |
| | M A P L A E V G G F L G G L E G L G Q P G A A Q L A H |
| 81 | CCTGCACGGCATCCTGCGCCGCCGGCAGCTCTATTGCCGCACCGGCTTCCACCTGCAGATCCTGCCCGACGGCAGCGTGC |
| | L H G I L R R R Q L Y C R T G F H L Q I L P D G S V Q |
| 161 | AGGGCACCCGGCAGGACCACAGCCTCTTCGGTATCTTGGAATTCATCAGTGTGGCAGTGGGACTGGTCAGTATTAGAGGT |
| | G T R Q D H S L F G I L E F I S V A V G L V S I R G |
| 241 | GTGGACAGTGGTCTCTATCTTGGAATGAATGACAAAGGAGAACTCTATGGATCAGAGAAACTTACTTCCGAATGCATCTT |
| | V D S G L Y L G M N D K G E L Y G S E K L T S E C I F |
| 321 | TAGGGAGCAGTTTGAAGAGAACTGGTATAACACCTATTCATCTAACATATATAAACATGGAGACACTGGCCGCAGGTATT |
| | R E Q F E E N W Y N T Y S S N I Y K H G D T G R R Y F |
| 401 | TTGTGGCACTTAACAAAGACGGAACTCCAAGAGATGGCGCCAGGTCCAAGAGGCATCAGAAATTTACACATTTCTTACCT |
| | V A L N K D G T P R D G A R S K R H Q K F T H F L P |
| 481 | AGACCAGTGGATCCAGAAAGAGTTCCAGAATTGTACAAGGACCTACTGATGTACACTTAG |
| | R P V D P E R V P E L Y K D L L M Y T |

TABLE 31

View DNA Sequence Analysis of 250059669
Translated Protein - Frame: 2 - Nucleotide 2 to 466

| | |
|---|---|
| 1 | CACCAGATCTATCCTGCGCCGCCGGCAGCTCTATTGCCGCACCGGCTTCCACCTGCAGATCCTGCCCGACGGCAGCGTGC |
| | T R S I L R R R Q L Y C R T G F H L Q I L P D G S V Q |
| 81 | AGGGCACCCGGCAGGACCACAGCCTCTTCGGTATCTTGGAATTCATCAGTGTGGCAGTGGGACTGGTCAGTATTAGAGGT |
| | G T R Q D H S L F G I L E F I S V A V G L V S I R G |
| 161 | GTGGACAGTGGTCTCTATCTTGGAATGAATGACAAAGGAGAACTCTATGGATCAGAGAAACTTACTTCCGAATGCATCTT |
| | V D S G L Y L G M N D K G E L Y G S E K L T S E C I F |
| 241 | TAGGGAGCAGTTTGAAGAGAACTGGTATAACACCTATTCATCTAACATATATAAACATGGAGACACTGGCCGCAGGTATT |
| | R E Q F E E N W Y N T Y S S N I Y K H G D T G R R Y F |
| 321 | TTGTGGCACTTAACAAAGACGGAACTCCAAGAGATGGCGCCAGGTCCAAGAGGCATCAGAAATTTACACATTTCTTACCT |
| | V A L N K D G T P R D G A R S K R H Q K F T H F L P |
| 401 | AGACCAGTGGATCCAGAAAGAGTTCCAGAATTGTACAAGGACCTACTGATGTACACTGTCGACGGC |
| | R P V D P E R V P E L Y K D L L M Y T V D G |

[image]

### 6.19. Example 19

### Expression of CG53135

Several different expression constructs were generated to express CG53135 proteins (Table 33). The CG53135-05 construct, a codon-optimized, phage-free construct encoding the full-length gene (construct #3 in Table 3), was expressed in E. coli BLR (DE3), and the purified protein product was used in toxicology studies and clinical trials.

TABLE 33
Constructs Generated to Express CG53135

| Con- | | | | |
|---|---|---|---|---|
| struct | Construct Description | Construct Diagram | | |
| 1a | NIH 3T3 cells were transfected with | CG53135-01 | V5 | His |

| | pFGF-20, which incorporates an epitope | | | | |
|---|---|---|---|---|---|
| | tag (V5) and a polyhistidine tag into | | | | |
| | the carboxyterminus of the CG53135-01 | | | | |
| | protein in the pcDNA3.1 vector (Invitrogen) | | | | |
| 1b | Human 293-EBNA embryonic kidney cells | IgK | CG53135-01 | V5 | His |

| | or NIH 3T3 cells were transfected with | | | |
|---|---|---|---|---|
| | CG53135-01 using pCEP4 vector (Invitrogen) | | | |
| | containing an IgK signal sequence, | | | |
| | multiple cloning sites, a V5 epitope tag, | | | |
| | and a polyhistidine tag | | | |
| 2 | E. coli BL21 cells were transformed with | His | T7 | CG53135-01 |

| | CG53135-01 using pETMY vector (CuraGen | |
|---|---|---|
| | Corporation) containing a polyhistidine | |
| | tag and a T7 epitope tag (this construct | |
| | is also referred to as E. coli/pRSET) | |
| 3 | E. coli BLR (DE3) cells (NovaGen) were | CG53135-05 |
| | transformed with CG53135-05 (full-length, | |
| | codon-optimized) using pET24a vector | |
| | (NovaGen) | |
| 4 | E. coli BLR (DE3) cells (NovaGen) were | CG53135-02 (deletion mutant) |

transformed with CG53135 (deletion of
amino acids 2-54, codon-optimized)
using pET24a vector (NovaGen)

In one construct, CG53135-01 (the full-length CG53135 gene) was cloned as a Bgl II-Xho I fragment into the Bam HI-Xho I sites in mammalian expression vector, pcDNA3.1V5His (Invitrogen Corporation, Carlsbad, Calif.). The resultant construct, pFGF-20 (construct 1a) has a 9 amino acid V5 tag and a 6 amino acid histidine tag (His) fused in-frame to the carboxy-terminus of CG53135-01. These tags aid in the purification and detection of CG53135-01 protein. After transfection of pFGF-20 into murine NIH 3T3 cells, CG63135-01 protein was detected in the conditioned medium using an anti-V5 antibody (Invitrogen, Carlsbad, Calif.).

The full-length CG53135-01 gene was also cloned as a Bgl II-Xho I fragment into the Bam HI-Xho I sites of mammalian expression vector pCEP4/Sec (CuraGen Corporation). The resultant construct, plgK-FGF-20 (construct 1b) has a heterologous immunoglobulin kappa (IgK) signal sequence that could aid in secretion of CG53135-01. After transfection of plgK-FGF-20 into human 293 EBNA cells (Invitrogen, Carlsbad, Calif.; catalog #R620-07), CG53135-01 was detected in the conditioned medium using an anti-V5 antibody.

In order to increase the yield of CG53135 protein, a Bgl II-Xho I fragment encoding the full-length CG53135-01 gene was cloned into the Bam HI-Xho I sites of E. coli expression vector, pETMY (CuraGen Corporation). The resultant construct, pETMY-FGF-20 (construct 2) has a 6 amino acid histidine tag and a T7 tag fused in-frame to the amino terminus of CG53135. After transformation of pETMY-FGF-20 into BL21 E. coli (Novagen, Madison, Wis.), followed by T7 RNA polymerase induction, CG53135-01 protein was detected in the soluble fraction of the cells.

In order to express CG53135 without tags, CG53135-05 (a codon-optimized, full-length FGF-20 gene) and CG53135-02 (a codon-optimized deletion construct of FGF-20, with the N-terminal amino acids 2-54 removed) were synthesized. For the full-length construct (CG53135-05), an Nde I restriction site (CATATG) containing the initiator codon was placed at the 5′ end of the coding sequence. At the 3′ end, the coding sequence was followed by 2 consecutive stop codons (TAA) and a Xho restriction site (CTCGAG). The synthesized gene was cloned into pCRScript (Stratagene, La Jolla, Calif.) to generate pCRScript-CG53135. An Nde I-Xho I fragment containing the codon-optimized CG53135 gene was isolated from the pCRscript-CG53135 and subcloned into Nde I-Xho I-digested pET24a to generate pET24a-CG53135 (construct 3). The full-length, codon-optimized version of CG53135 is referred to as CG53135-05.

To generate a codon-optimized deletion construct for CG53135, oligonucleotide primers were designed to amplify the deleted CG53135 gene from pCRScript-CG53135. The forward primer contained an Nde I site (CATATG) followed by coding sequence starting at amino acid 55. The reverse primer contained a HindIII restriction site. A single PCR product of approximately 480 base pairs was obtained and cloned into pCR2.1 vector (Invitrogen) to generate pCR2.1-CG53135del. An Nde I-Hind III fragment was isolated from pCR2.1-53135del and subcloned into Nde I-Hind III-digested pET24a to generate pET24a-CG53135-02 (construct 4).

The plasmids, pET24a-CG53135-05 (construct 3) and pET24a-CG53135-02 (construct 4) have no tags. Each vector was transformed into E. coli BLR (DE3), induced with isopropyl thiogalactopyranoside. Both the full-length and the N-terminally truncated CG53135 protein was detected in the soluble fraction of cells.

### 6.20. Example 20

### Protein Expression and Purification

The pET24a-CG53135-05 (construct 3, see Example 18) was expressed in Escherichia coli BLR (DE3), purified to homogeneity, and characterized by standard protein chemistry techniques.

Fermentation and Primary Recovery Recombinant

CG53135-05 was expressed using Escherichia coli BLR (DE3) cells (Novagen). These cells were transformed with full length, codon optimized CG53135-05 using pET24a vector (Novagen). A Manufacturing Master Cell Bank (MMCB) of these cells was produced and qualified. The fermentation and primary recovery processes were performed at the 100 L (i.e., working volume) scale reproducibly.

Seed preparation was started by thawing and pooling of 1-6 vials of the MMCB and inoculating 4-7 shake flasks each containing 750 mL of seed medium. At this point, 3-6 L of inoculum was transferred to a production fermentor containing 60-80 L of start-up medium. The production fermentor was operated at a temperature of 37° C. and pH of 7.1. Dissolved oxygen was controlled at 30% of saturation concentration or above by manipulating agitation speed, air sparging rate and enrichment of air with pure oxygen. Addition of feed medium was initiated at a cell density of 30-40 AU (600 nm) and maintained until end of fermentation. The cells were induced at a cell density of 40-50 AU (600 nm) using 1 mM isopropyl-beta-D-thiogalactoside (IPTG) and CG53135-05 protein was produced for 4 hours post-induction. The fermentation was completed in 10-14 hours and about 100˜110 L of cell broth was concentrated using a continuous centrifuge. The resulting cell paste was stored frozen at −70° C.

The frozen cell paste was suspended in lysis buffer (containing 3M urea, final concentration) and disrupted by high-pressure homogenization. The cell lysate was clarified using continuous flow centrifugation. The resulting clarified lysate was directly loaded onto a SP-sepharose Fast Flow column equilibrated with SP equilibration buffer (3 M urea, 100 mM sodium phosphate, 20 mM sodium chloride, 5 mM EDTA, pH 7.4). CG53135-05 protein was eluted from the column using SP elution buffer (100 mM sodium citrate, 1 M arginine, 5 mM EDTA, pH 6.0). The collected material was then diluted with an equal volume of SP elution buffer. After thorough mixing, the SP Sepharose FF pool was filtered through a 0.2 μm PES filter and frozen at −80° C.

Purification of the Drug Substance

The SP-sepharose Fast Flow pool was precipitated with ammonium sulfate. After overnight incubation at 4° C., the precipitate was collected by bottle centrifugation and subsequently solubilized in Phenyl loading buffer (100 mM sodium citrate, 500 mM L-arginine, 750 mM NaCl, 5 mM EDTA, pH 6.0). The resulting solution was filtered through a 0.45 uM PES filter and loaded onto a Phenyl-sepharose HP column. After washing the column, the protein was eluted with a linear gradient with Phenyl elution buffer (100 mM sodium citrate, 500 mM L-arginine, 5 mM EDTA, pH 6.0). The Phenyl-sepharose HP pool was filtered through a 0.2 μm PES filter and frozen at −80° C. in 1.8 L aliquots.

Formulation and Fill/Finish

Four batches of purified drug substance were thawed for 24-48 hours at 2-8° C. and pooled into the collection tank of tangential flow ultrafiltration (TFF) equipment. The pooled drug substance was concentrated ˜5-fold via TFF, followed by about 5-fold diafiltration with the formulation buffer (40 mM sodium acetate, 0.2 M L-arginine, 3% glycerol). This buffer-exchanged drug substance was concentrated further to a target concentration of >10 mg/mL. Upon transfer to a collection tank, the concentration was adjusted to ˜10 mg/mL with formulation buffer. The formulated drug product was sterile-filtered into a sterile tank and aseptically filled (at 10.5 mL per 20 mL vial) and sealed. The filled and sealed vials were inspected for fill accuracy and visual defects. A specified number of vials were drawn and labeled for release assays, stability studies, safety studies, and retained samples. The remaining vials were labeled for the clinical study, and finished drug product was stored at −80±15° C.

The finished drug product is a sterile, clear, colorless solution in single-use sterile vials for injection. CG53135-05 E. coli purified product was formulated at a final concentration of 8.2 mg/ml.

The final purified protein product described above was analyzed using techniques such as Liquid Chromatography, Mass spectrometry and N-terminal sequencing. Such analyses indicate that the final purified protein product includes some truncated form of FGF-20 (e.g., CG53135-13 (SEQ ID NO:24), CG53135-15 (SEQ ID NO:28), CG53135-16 (SEQ ID NO:30), and CG53135-17 (SEQ ID NO:32)) in addition to the full length FGF-20, and a protein consisting of amino acids 3-211 (CG53135-13, SEQ ID NO:24) of FGF-20 constitutes the majority of the final purified protein product.

All the variants/fragments in the final purified product have high activity in the proliferation assays. Thus these variants/fragments are expected to have same utility as that of FGF-20. For the purpose of convenience, the term “CG53135-05 E. coli purified product” is used herein to refer to a purified protein product from E. coli expressing a CG53135-05 construct. For example, a CG53135-05 E. coli purified product may contain a mixture of the full length CG53135-05 protein (SEQ ID NO:2), CG53135-13 (SEQ ID NO:24), CG53135-15 (SEQ ID NO:28), CG53135-16 (SEQ ID NO:30), and CG53135-17 (SEQ ID NO:32), with the majority of the content being CG53135-13 (SEQ ID NO:24).

RP-HPLC Assay: Peak Identification

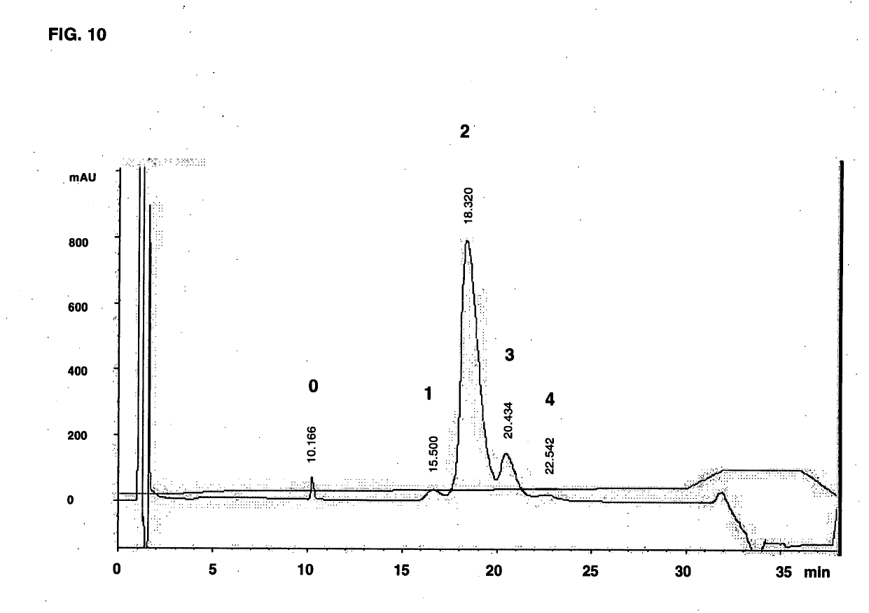

Purified drug substance was further analyzed by reversed-phase high-performance liquid chromatography (RP-HPLC) with both UV and electrospray mass spectrometric detection. Purified protein was loaded onto a Protein C4 column (Vydac, 5 μm, 150 mm×4.6 mm) using a standard HPLC system in a mobile phase containing water, acetonitrile and trifluoroacetic acid. The elution gradient for this method was modified to resolve four distinct chromatographic peaks eluting at 26.6, 27.3, 28.5 and 30.0 min respectively (FIG. 10). These peaks were characterized by electrospray mass spectrometry. As can be observed from the chromatograms, the four equipotent peaks are present in the purified final product.

The identities of each peak from the RP-HPLC separation are indicated in Table 34.

TABLE 34
Identity of peaks from the RP-HPLC separation of CG53135-05 E. coli purified
product based upon accurate molecular weight determination

| | | MOLECULAR | | PREDICTED |
|---|---|---|---|---|
| PEAK | RETENTION | WEIGHT | ASSIGNMENT | MOLECULAR |
| # | TIME (MIN) | OBSERVED | (RESIDUE #) | WEIGHT |
| 1 | 26.6 | 21329.2 | 24-211 | 21329.2 |
| 1 | 26.6 | 22185.1 | 15-211 | 22185.1 |
| 1 | 26.6 | 22412.4 | 12-211 | 22412.4 |
| 2 | 27.3 | 23296.5 | 3-211 | 23296.4 |
| 3 | 28.5 | 23498.9 | 1-211 | 23498.7 |
| 4 | 30.0 | 23339.3 | 3-211 | 23339.4 |
| | | | (CARBAMYLATED) | |
| 4 | 30.0 | 23539.7 | 1-211 | 23539.7 |
| | | | (CARBAMYLATED) | |

Edman Sequencing and Total Amino Acid Analysis

The experimental N-terminal amino acid sequence of the reference standard of the purified product, DEV1 0, was determined qualitatively. The reference standard was resolved by SDS-AGE and electrophoretically transferred to a polyvinylidenefluoride membrane; the Coomassie-stained ˜23 kDa major band corresponding to each reference standard was excised from the membrane and analyzed by an automated Edman sequencer (Procise, Applied Biosystems, Foster City, Calif.). The major sequences is shown in Table 35 below. The predominant sequence was corresponded to residues 3-20 in the theoretical N-terminal sequence of CG53135-05.

TABLE 35
Edman sequencing data for the first 20 amino acids
of CG53135-05 E. coli purified product

| Theoretical Residue Position | Amino Acid Residue |
|---|---|
| 3 | Pro |
| 4 | Leu |
| 5 | Ala |
| 6 | Glu |
| 7 | Val |
| 8 | Gly |
| 9 | Gly |
| 10 | Phe |
| 11 | Leu |
| 12 | Gly |
| 13 | Gly |
| 14 | Leu |
| 15 | Glu |
| 16 | Gly |
| 17 | Leu |
| 18 | Gly |
| 19 | Gln |
| 20 | Gln |

The experimental amino acid composition of the DEV10 reference standard was determined. Quadruplicate samples of the reference standard were hydrolyzed for 16 hours at 115° C. in 100 μL of 6 N HCl, 0.2% phenol containing 2 nmol norleucine as an internal standard. Samples were dried in a Speed Vac Concentrator and dissolved in 100 μL sample buffer containing 2 nmol homoserine as an internal standard. The amino acids in each sample were separated on a Beckman Model 7300 amino acid analyzer. Note that Cys and trp are destroyed during acid hydrolysis of the protein. Asn and gln are converted to asp and glu, respectively, during acid hydrolysis and thus their totlas are reported as asx and glx. Mat and his were both unresolved in this procedure.

TABLE 36
Quantitive amino acid analysis for CG53135-05
E. coli purified product

| | Mole Percent |
|---|---|
| Amino Acid Residue | DEV10 |
| asx | 7.1 |
| thr | 4.0 |
| ser | 6.3 |
| glx | 12.2 |
| pro | 6.0 |
| gly | 14.4 |
| ala | 5.8 |
| val | 5.3 |
| ile | 3.5 |
| leu | 13.6 |
| tyr | 4.6 |
| phe | 5.2 |
| lys | 3.7 |
| arg | 8.5 |

Peptide Mapping

CG53135-05 E. coli purified product (25 mg) was denatured and reduced in urea and dithiothreitol at 50° C. and then alkylated with iodoacetate. After lowering the concentration of urea, the samples were treated with trypsin for 40 hours at 20° C. The resulting peptide fragments were separated by RP-HPLC (using a C-18 column with an acetonitrile gradient in trifluoroacetate) to obtain a peptide map (FIGS. 11A and 11B). The chromatogram in FIG. 11A is consistent with the 20 peptides expected from the digestion of CG53135-05 with trypsin, and the chromatogram in FIG. 11B reveals a single peak as expected for the single tryptophan residue in CG53135-05.

Bioassay

The biological activity of CG53135-05 related species collected from the 4 peaks identified by LC and MS was measured by treatment of serum-starved cultured NIH 3T3 murine embryonic fibroblast cells with various doses of the isolated CG53135-05 related species and measurement of incorporation of bromodeoxyuridine (BrdU) during DNA synthesis. For this assay, cells were cultured in Dulbecco's modified Eagle's medium supplemented with 10% fetal bovine serum. Cells were grown in 96-well plates to confluence at 37° C. in 10% CO₂/air and then starved in Dulbecco's modified Eagle's medium for 24-72 hours. CG53135-05-related species were added and incubated for 18 hours at 37° C. in 10% CO₂/air. BrdU (10 mM final concentration) was added and incubated with the cells for 2 hours at 37° C. in 10% CO₂/air. Incorporation of BrdU was measured by enzyme-linked immunosorbent assay according to the manufacturer's specifications (Roche Molecular Biochemicals, Indianapolis, Ind.).

Peak 4 was not included in this assay since insufficient material was collected (Peak 4 is less than 3% of the total peak area for CG53135-05). CG53135-05 and material collected from all 3 remaining fractions (i.e., Peak 1, 2, and 3) induced DNA synthesis in NIH 3T3 mouse fibroblasts in a dose-dependent manner (Table 37). The PI₂₀₀ was defined as the concentration of protein that resulted in incorporation of BrdU at 2 times the background. CG53135-05 and CG53135-05 related species recovered from all 3 measurable peaks demonstrated similar biological activity with a PI₂₀₀ of 0.7-11 ng/mL (Table 37).

TABLE 37
BIOLOGICAL ACTIVITY OF CG53135-05 (DEV10):
INDUCTION OF DNA SYNTHESIS
PI₂₀₀ (ng/mL)

| CG53135-05 (DEV10 | Peak 1 | Peak 2 | Peak 3 |
|---|---|---|---|
| 1.0 | 0.7 | 11 | 8.6 |

### 6.21. Example 21

### Wound Repair Test

In vitro cell culture: The human colon cancer cell line Caco2, HT29 and THP-1 cells were obtained from the American Type Culture Collection (Rockville, Md.), HT-29 MTX were provided by Dr. Lesuffier, INSERM, Dillejuis, France. These cell lines (Caco2, HT-29 and HT-29MTX) were grown as described previously. THP-1 cell lines were grown in RPMI-1640 medium (Life Technologies, Gaithersburg, Md.) with 10% fetal bovine serum, 100 units/ml of antibiotics/antimycotics (Life Technologies, Gaithersburg, Md.).

An in vitro healing assay was performed using a modified method. Briefly, reference lines were drawn horizontally across the outer bottom of 24-well plates. HT-29 and Caco-2 cells were seeded and grown to confluence, then incubated with media containing 0.1% FBS for 24 hours.

Linear wounds were made with a sterile plastic pipette tip perpendicular to the lines on the bottom of the well. Isolated FGF-20 protein (100 ng/ml) was then added. The size of the wound was measured at three predetermined locations at various times after wounding (0, 6, 20 and 24 hours). The closure of the wounds was measured microscopically at 20× magnification over time, and the mean percentage of wound closure was calculated relative to baseline values (time 0). To investigate whether the effect of FGF-20 on cell restitution is involved with TGF-α and ITF pathway, anti-TGFα antibody (R&Dsystem, Minneapolis, Minn.) and polyclonal anti-ITF antibody (a gift from D K Podolsky, Harvard Medical School, Boston, Mass.) were used.

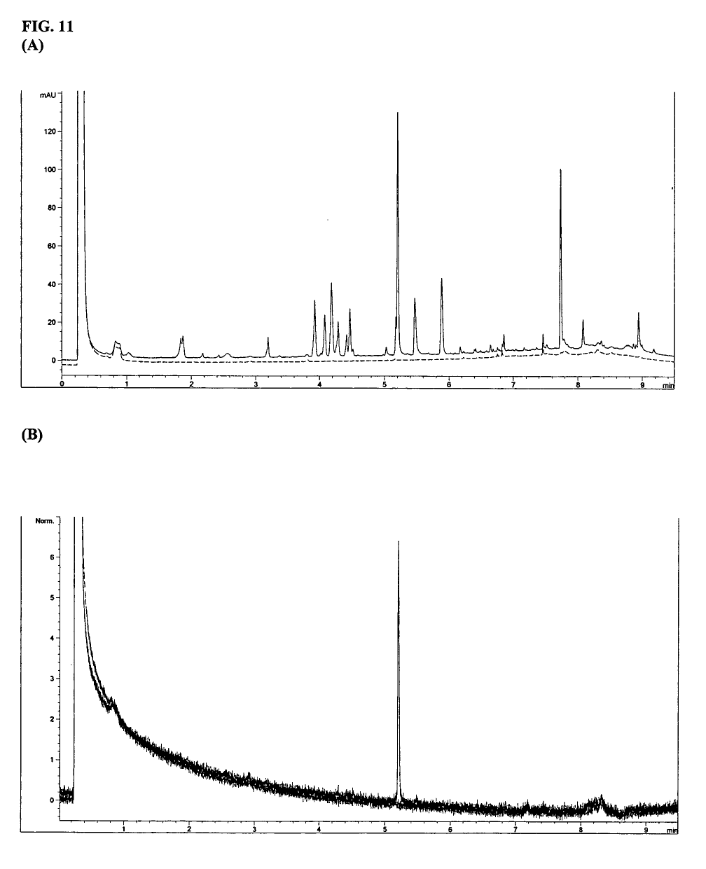

FIG. 12 show the effect of FGF-20 in the closure of wounds in various human cell lines. There is a dose dependent increase in the effectiveness of FGF-20 in the closure of wounds in all the cell lines tested, demonstrating the role of FGF-20 in wound repair.

### 6.22. Example 22

### Cellular Proliferation Responses with CG53135 (Studies L-117.01 and L-117.02)

Experiments were performed to evaluate the proliferative response of representative cell types to CG53135, e.g., a full-length tagged variant (CG53135-01), a deletion variant (CG53135-02), and a full-length codon-optimized untagged variant (CG53135-05).

Materials and Methods:

Heterologous Protein Expression: CG53135-01 (batch 4A and 6) was used in these experiments. Protein was expressed using Escherichia coli (E. coli), BL21 (Novagen, Madison, Wis.), transformed with full-length CG53135-01 in a pETMY-hFGF20X/BL21 expression vector. Cells were harvested and disrupted, and then the soluble protein fraction was clarified by filtration and passed through a metal chelation column. The final protein fraction was dialyzed against phosphate buffered saline (PBS) plus 1 M L-arginine. Protein samples were stored at −70° C.

CG53135-02 (batch 1 and 13) was also used in these experiments. Protein was expressed in E. coli, BLR (DE3) (Novagen), transformed with the deletion variant CG53135-02 inserted into a pET24a vector (Novagen). A research cell bank (RCB) was produced and cell paste containing CG53135-02 was produced by fermentation of cells originating from the RCB. Cell membranes were disrupted by high-pressure homogenization, and lysate was clarified by centrifugation. CG53135-02 was purified by ion exchange chromatography. The final protein fraction was dialyzed against the formulation buffer (100 mM citrate, 1 mM ethylenediaminetetraacetic acid (EDTA), and 1 M L-arginine).

CG53135-05, DEV10, which were also used in these experiments, was prepared by Cambrex Biosciences (Hopkinton, Mass.) according to the method described in Section 6.20, supra.

BrdU Incorporation: proliferative activity was measured by treatment of serum-starved cultured cells with a given agent and measurement of BrdU incorporation during DNA synthesis. Cells were cultured in respective manufacturer recommended basal growth medium supplemented with 10% fetal bovine serum or 10% calf serum as per manufacturer recommendations. Cells were grown in 96-well plates to confluence at 37+ C. in 10% CO₂/air (to subclonfluence at 5% CO₂ for dedifferentiated chondrocytes and NHOst). Cells were then starved in respective basal growth medium for 24-72 hours. CG53135 protein purified from E. coli or pCEP4/Sec or pCEP4/Sec-FGF 20× enriched conditioned medium was added (10 μL/100 μL of culture) for 18 hours. BrdU (10 μM final concentration) was then added and incubated with the cells for 5 hours. BrdU incorporation was assayed according to the manufacturer's specifications (Roche Molecular Biochemicals, Indianapolis, Ind.).

Growth Assay: growth activity was obtained by measuring cell number following treatment of cultured cells with a given agent for a specified period of time. In general, cells grown to ˜20% confluency in 6-well dishes were treated with basal medium supplemented with CG53135 or control, incubated for several days, trypsinized and counted using a Coulter Z1 Particle Counter.

Results:

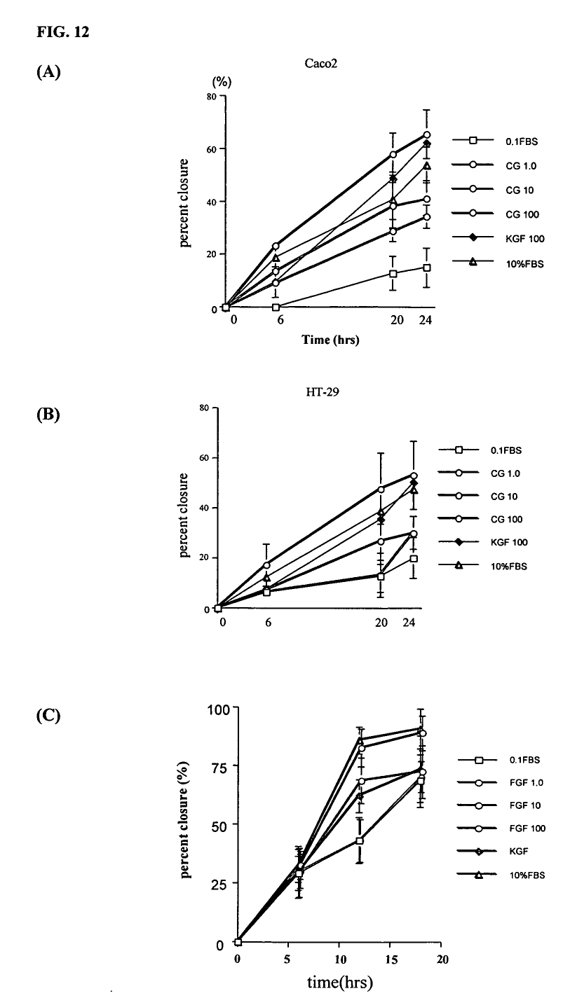

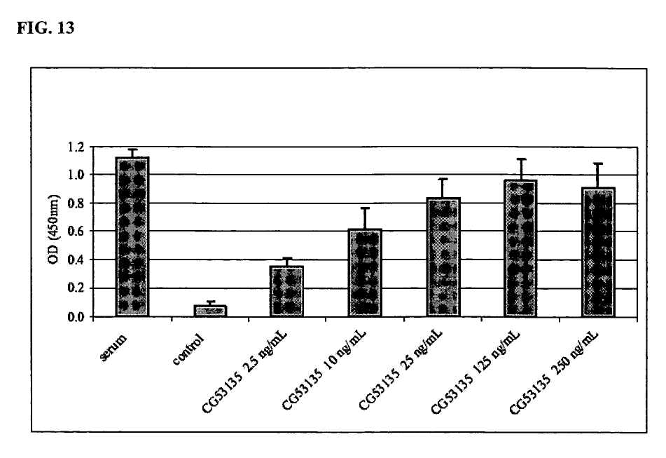

Proliferation in Mesenchymal Cells: to determine if recombinant CG53135 could stimulate DNA synthesis in fibroblasts, a BrdU incorporation assay was performed using CG53135-01 treated NIH 3T3 murine embryonic lung fibroblasts. Recombinant CG53135-01 induced DNA synthesis in NIH 3T3 mouse fibroblasts in a dose-dependent manner (FIG. 13). DNA synthesis was generally induced at a half maximal concentration of ˜10 ng/mL. In contrast, treatment with vehicle control purified from cells did not induce any DNA synthesis.

CG53135-01 also induced DNA synthesis in other cells of mesenchymal origin, including CCD-1070Sk normal human foreskin fibroblasts, MG-63 osteosarcoma cell line, and rabbit synoviocyte cell line, HIG-82. In contrast, CG53135-01 did not induce any significant increase in DNA synthesis in primary human osteoblasts (NHOst), human pulmonary artery smooth muscle cells, human coronary artery smooth muscle cells, human aorta smooth muscle cells (HSMC), or in mouse skeletal muscle cells.

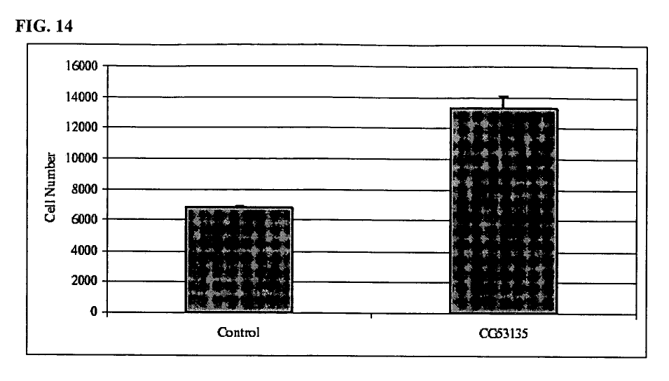

To determine if recombinant CG53135-01 sustained cell growth, NIH 3T3 cells were cultured with 1 μg CG53135-01 or control for 48 hours and then counted (FIG. 14). CG53135 induced an approximately 2-fold increase in cell number relative to control in this assay. These results show that CG53135 acts as a growth factor.

Proliferation of Epithelial Cells: to determine if recombinant CG53135 can stimulate DNA synthesis and sustain cell growth in epithelial cells, a BrdU incorporation assay was performed in representative epithelial cell lines treated with CG53135. Cell counts following protein treatment were also determined for some cell lines.

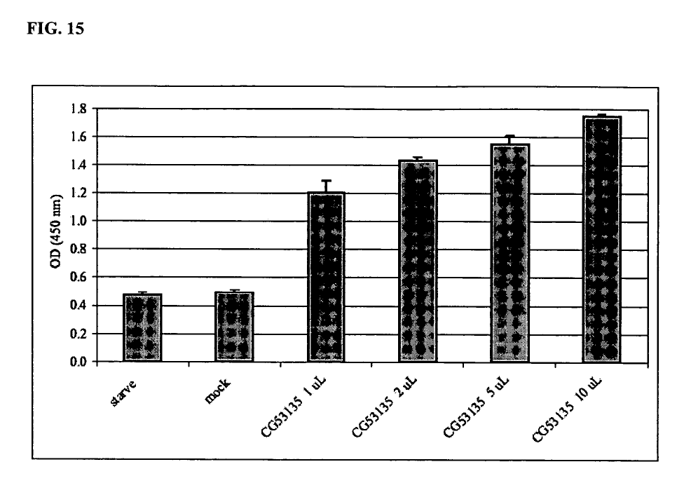

CG53135 was found to induce DNA synthesis in the 786-O human renal carcinoma cell line in a dose-dependent manner (FIG. 15). In addition, CG53135-01 induced DNA synthesis in other cells of epithelial origin, including CCD 1106 KERTr human keratinocytes, Balb MK mouse keratinocytes, and breast epithelial cell line, B5589.

Proliferation of Hematopoietic Cells: no stimulatory effect on DNA synthesis was observed upon treatment of TF-1, an erythroblastic leukemia cell line with CG53135-01. These data suggest that CG53135-01 does not induce proliferation in cells of erythroid origin. In addition, Jurkat, an acute T-lymphoblastic leukemia cell line, did not show any response when treated with CG53135-01, whereas a robust stimulation of BrdU incorporation was observed with serum treatment.

Effects of CG53135 on Endothelial Cells: protein therapeutic agents may inhibit or promote angiogenesis, the process through which endothelial cells differentiate into capillaries. Because CG53135 belongs to the fibroblast growth factor family, some members of which have angiogenic properties, the antiangiogenic or pro-angiogenic effects of CG53135 on endothelial cell lines were evaluated. The following cell lines were chosen because they are cell types used in understanding angiogenesis in cancer: HUVEC (human umbilical vein endothelial cells), BAEC (bovine aortic endothelial cells), HMVEC-d (human endothelial, dermal capillary). These endothelial cell types undergo morphogenic differentiation and are representative of large vessel (HUVEC, BAEC) as well as capillary endothelial cells (HMVEC-d).

CG53135-01 treatment did not alter cell survival or have stimulatory effects on BrdU incorporation in human umbilical vein endothelial cells, human dermal microvascular endothelial cells or bovine aortic endothelial cells. Furthermore, CG53135-01 treatment did not inhibit tube formation, an important event in formation of new blood vessels, in HUVECS. This result suggests that CG53135 does not have anti-angiogenic properties. Finally, CG53135-01 had no effect on VEGF induced cell migration in HUVECs, suggesting that it does no play a role in metastasis.

The above described experiments were also performed using CG53135-02 and CG53135-05 protein products, and the results are summarized in the Conclusion section below.

Conclusions

Recombinant CG53135-01 induces a proliferative response in mesenchymal and epithelial cells in vitro (i.e., NIH 3T3 mouse fibroblasts, CCD-1070 normal human skin fibroblasts, CCD-1 106 human keratinocytes, 786-O human renal carcinoma cells, MG-63 human osteosarcoma cells and human breast epithelial cells), but not in human smooth muscle, erythroid, or endothelial cells. Like CG53135-01, CG53135-02 and CG53135-05 also induce proliferation of mesenchymal and epithelial cells. In addition, CG53135-02 (but not CG53135-01 nor CG53135-05) induces proliferation of endothelial cells.

### 6.23. Example 23

### Production of Rabbit Polyclonal Anti-CG53135-01 Sera

Rabbit polyclonal anti-CG53135 sera were produced as follows: two female New Zealand White rabbits (identification numbers 2447 and 2448, age 8-12 wk, weight 4-5 lbs., Gingrich Animal Supply, Inc., Fredericksburg, Pa.) were immunized intradermally with 500 pg of CG53135-01 protein (batch 6) in complete Freund's adjuvant on 19 Jan. 2001. Boosters comprising 250 μg in incomplete Freund's adjuvant were given intradermally at 1 wk and subcutaneously at 2 and 4 wk. Five additional boosters (100-250 μg) were given every 4-6 wk; post-immunization sera were collected intermittently for approximately 31 wk; and rabbits were exsanguinated on 23 Aug. 2001 for final sera collection. Pre-immunization sera was collected 4 d prior to the primary immunization.

### 6.24. Example 24

### Purification of Pooled Rabbit Polyclonal Anti-CG5313501 Antibody

The IgG fraction was purified from rabbits #2447 and #2448 post-immunization serum (collected approximately 10 week post-primary immunization, 4.2.01) by protein G-Sepharose chromatography according to the manufacturer's instructions (Amersham Pharmacia Biotech, Uppsala, Sweden). Briefly, the 5 mL column was washed with 50 mL of manufacturer's binding buffer; 5 mL of rabbit serum was applied to the column; and the column was washed again with 25 mL of manufacturer's binding buffer. The IgG fractions were eluted with 2-5 column volumes of manufacturer's elution buffer, and the purified fractions were buffer exchanged by PBS dialysis overnight at 4° C. The presence of IgG in the protein G-purified fraction was confirmed by Western blot analysis. The concentration of the protein G-purified IgG fraction (i.e., rabbit anti-CG53135-01 antibody) was 4.46 mg/mL (batch #4) and 10.4 mg/mL (batch #5) for rabbits #2447 and #2448 respectively as determined by Bradford protein measurement method. To keep the ratio of the pooled polyclonal antibodies identical to previous batch #3, the concentrations of batch #4 and #5 were diluted to obtain 3.4 mg/mL and 4.4 mg/mL respectively (identical to batch #1 and #2). The pooled rabbit polyclonal anti-CG53135-01 antibody was then obtained by combining equal volumes of each rabbit IgG fraction. The concentration of this pooled antibody was the mean of the two fraction concentrations and was 3.9 mg/mL. This preparation was assayed for reactivity to CG53135-05 in an indirect ELISA.

### 6.25. Example 25

### Protein-G Purification of Rabbit Anti-CG53135 Polyclonal Antibody

Rabbit polyclonal anti-CG53135 sera from rabbit #2448 were titered and of 11 bleeds tested, 4 were chosen to be individually purified on 4 separate Hi Trap Protein G HP 1 mL protein-G columns (Amersham Biosciences, #17-0404-01). The 4 bleeds chosen were: May 7, 2001, Jun. 4, 2001, Aug. 13, 2001, and the termination bleed—Aug. 20, 2001. A summary of the purification steps follow:
Clarified crude antisera prior to putting on column by diluting sample 1:5 in binding buffer (20 mM sodium phosphate, pH 7.0-7.3), or 1 mL crude serum and 4 mL binding buffer.
Equilibrated column with 10 column volumes of binding buffer (20 mM sodium phosphate, pH 7.0-7.3) at a rate of approximately 1mL/min. Discarded buffer flow-through.
Applied prediluted crude sample to column with a 5 mL syringe, and collected in 5×1 mL fractions in 1.5 mL Eppendorf tubes labeled “FT1” to “FT5” (for 5 consecutive flow-through tubes). Saved fractions.
Washed column with 7 column volumes of binding buffer. Note: Instruction booklet gives the option of collecting from 5-10 column volumes, so arbitrarily chose 7. Collected and saved 7 tubes at approximately 1 mL/fraction.
Eluted sample with 5 column volumes of Elution buffer (0.1M glycine, pH 2.85). Collected and saved at approximately 1 mL/fraction.
Added 1/10 volume of 1 M Tris, pH 8.0 (Ambion, #9856). Most of the eluate collected was a 1 mL fraction, so added ˜100 uL of 1 M Tris to each tube.
Later in the day, washed column(s) with 5 column volumes of 20% ethanol. Stored in 20% ethanol, wrapped column(s) in parafilm, and retired columns upright at 4° C.

The purified fractions were run on two Coomassie gels (4-20% Tris-Glycine 15well gels, #EC60255BOX, Invitrogen) to identify the presence and purity of antibody. For all 4 bleeds, it was evident that the purified pAb consistently eluted from fractions #2-#5, with the heaviest staining at fraction #2. Consequently, for each of the 4 bleeds, fractions 2 through 5 were pooled (˜4mL total per bleed) and all 4 pools were dialyzed against 20 mM sodium phosphate pH 7.3 twice-once overnight at 4° C. and once for 2 hours at 4° C. the following morning. (Slide-A-Lyzer 10K MWCO dialysis cassettes, 3-15 mL sample volume, #66410, Pierce). The concentration of the protein G-purified IgG fractions (i.e., rabbit anti-CG53135-01 pAbs) were determined by BCA Protein Assay (Pierce, #23225) as noted below:

TABLE 38

| pAb | Conc | Final vol | |
|---|---|---|---|
| bleed # | (mg/mL) | (mL) | Amt (mg) |
| Bleed 1 | 2.2 | 3.9 | 8.58 |
| Bleed 2 | 3.0 | 3.8 | 11.4 |
| Bleed 3 | 3.35 | 3.6 | 12.06 |
| Bleed 4 | 3.48 | 3.5 | 12.18 |

Two days later, each IgG pool was sterile-filtered through a 0.2 uM filtration membrane, aliquoted at ˜1 mL/vial, and stored at 4° C.

A week later, OD280 readings were done on each of the 4 bleeds (bovine IgG standard), and were compared with the prior week's BCA data. See below for a comparison.

TABLE 39

| pAb | pAb/bleed | BCA conc | OD280 conc |
|---|---|---|---|
| bleed# | date | (mg/mL) | (mg/mL) |
| 1 | 5/7/2001 | 2.20 | 1.822 |
| 2 | 6/4/2001 | 3.00 | 2.238 |
| 3 | 8/13/2001 | 3.35 | 2.576 |
| 4 | 8/20/2001 | 3.48 | 2.352 |

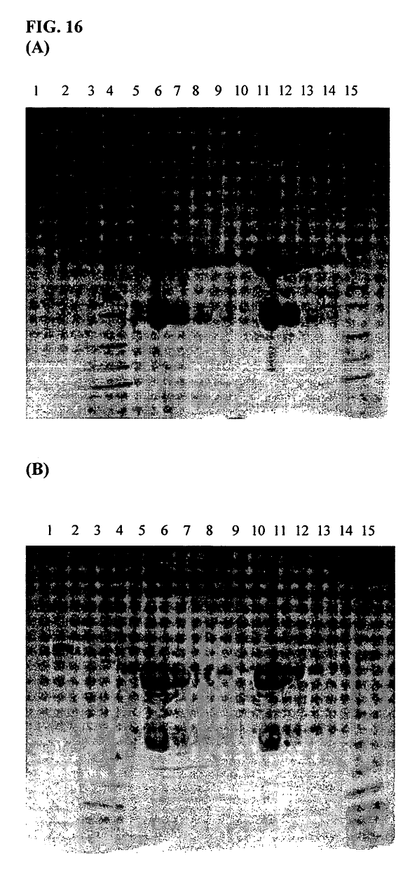

Fractions from purified IgG were analyzed under reducing conditions on Tris-Glycine SDS gels (4-20%). Twenty microliters from each sample were loaded and eluted at 200 V constant. Gels were stained with Simply Blue SafeStain (Invitrogen) (FIGS. 16(A) and (B)).

### 6.26. Example 26

### Quantitation of CG53135 in Biological Samples

CG53135 is detected in serum and plasma of rodent species such as mice, hamsters and rats as well as primate and human samples using an Enzyme Linked Immunosorbent Assays (ELISA). Briefly, a monoclonal antibody to CG53135-05 is immobilized on 96-well microtiter plates and CG53135-05 is captured from the biological matrix of the test species. Captured CG53135-05 is detected with the purified rabbit polyclonal antibody described above. The colorimetric signal is generated with a polyclonal donkey anti-rabbit horseradish peroxidase conjugate followed by addition of the chromogenic substrate, tetramethylbenzidine.

### 6.27. Example 27

### Modulation of Intestinal Crypt Cell Proliferation and Apoptosis by CG53135-05 Administration to Mice (Study N-342)

This study evaluated the effect of CG53135 on small intestinal crypt cell turnover in order to discriminate stem cell versus daughter cell effects, and to draw insights regarding the mode of action of CG53135 in syndromes associated with gastrointestinal stem cell damage (e.g., mucositis). Furthermore, the effect of CG53135 on stem cell radiosensitivity was also assessed. Protein concentrations in this example were measured by Bradford assay.

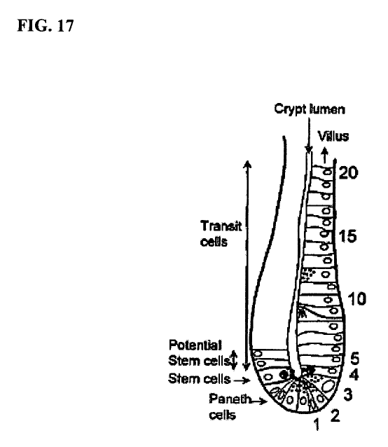

A “crypt” is a hierarchical structure with the stem cells towards the crypt base. As cells become more mature, they move progressively from the bottom of the crypt towards the top of the crypt. Therefore, changes that may be affecting stem cells versus their transit amplifying daughter cells can be detected by looking at changes in event frequency at each cell position. The cell positions are marked in FIG. 17. Thus, the effects of CG53135 on the crypt microarchitecture were analyzed in the context of crypt cellularity.

Experimental Design

Animals were sacrificed at various times after a single 12 mg/kg (Bradford, IP) dose of the CG53135-05 E. coli purified product. Just prior to sacrifice the mice were labeled with a single injection of bromodeoxyuridine to label S-phase cells and determine the effect of the drug on crypt cell proliferation/apoptosis. Two further groups of mice were used to assess effects on stem cell radiosensitivity. One group was treated with the CG53135-05 E. coli purified product (12 mg/kg, Bradford single injection, IP) and another group was injected with a placebo control. Twenty-four hours post injection, animals were irradiated with 1Gy X-ray (specifically to induce stem cell aptosis) followed by routine in vivo BrdU labeling. Animals were sacrificed 4.5 hours later (at time of peak apoptosis).

Mice were weighed and then dosed with the CG53135-05 E. coli purified product (12 mg/kg, Bradford, single injection, IP). Groups of 6 animals were sacrificed 0, 3, 6, 9,12, 24, 48 hours post injection with CG53135-05 E. coli purified product. All received a single injection of bromodeoxyuridine 40 minutes prior to sacrifice (see Table 40).

An additional two groups of 6 mice were used to assess the effects of CG53135-05 on stem cell radiosensitivity (groups 8 and 9, see Table 8). One group was treated with CG53135-05 (12 mg/kg Bradford, single injection, ip) and one group was injected with a placebo control. 24 hours post injection, animals were irradiated with 1 Gy X-ray and sacrificed 4.5 hours later.

TABLE 40
Study Design

| Group | Number of | | Treatment |
|---|---|---|---|
| Number | Animals | Treatment | Schedule* |
| 1 | 6 males | CG53135-05 E. coli | Injected and euthanize |
| | | purified product, | 3 hr later |
| | | 12 mg/kg, IP | 40 mg/kg BrdU 40 min prior |
| | | | to sacrifice |
| 2 | 6 males | CG53135-05 E. coli | Injected and euthanize |
| | | purified product, | 6 hr later |
| | | 12 mg/kg, IP | 40 mg/kg BrdU 40 min prior |
| | | | to sacrifice |
| 3 | 6 males | CG53135-05 E. coli | Injected and euthanize |
| | | purified product, | 9 hr later |
| | | 12 mg/kg, IP | 40 mg/kg BrdU 40 min prior |
| | | | to sacrifice |
| 4 | 6 males | CG53135-05 E. coli | Injected and euthanize |
| | | purified product, | 12 hr later |
| | | 12 mg/kg, IP | 40 mg/kg BrdU 40 min prior |
| | | | to sacrifice |
| 5 | 6 males | CG53135-05 E. coli | Injected and euthanize |
| | | purified product, | 24 hr later |
| | | 12 mg/kg, IP | 40 mg/kg BrdU 40 min prior |
| | | | to sacrifice |
| 6 | 6 males | CG53135-05 E. coli | Injected and euthanize |
| | | purified product, | 48 hr later |
| | | 12 mg/kg, IP | 40 mg/kg BrdU 40 min prior |
| | | | to sacrifice |
| 7 | 6 males | Untreated | 40 mg/kg BrdU 40 min prior |
| | | | to sacrifice |
| 8 | 6 males | CG53135-05 E. coli | Dose 24 hr prior to |
| | | purified product, | irradiation |
| | | 12 mg/kg, IP | Euthanize 4.5 hr post |
| | | IGy X ray | irradiation |
| | | | 40 mg/kg BrdU 40 min prior |
| | | | to sacrifice |
| 9 | 6 males | PBS, IP | Dose 24 hr prior to |
| | | 1Gy X-ray | irradiation |
| | | | Euthanize 4.5 hr post |
| | | | irradiation |
| | | | 40 mg/kg BrdU 40 min prior |
| | | | to sacrifice |

Intestinal Crypt Cell Proliferation and Apoptosis Modulation: Procedure

All S-phase dividing cells incorporate the injected Bromodeoxyuridine (BrdU) and hence were marked as cycling cells. Animals that were irradiated were placed, unanaesthetised, in a perspex jig and subjected to whole body radiation of 1 Gy X-ray at a dose rate of 0.7 Gy/min. This low level of radiation induced apoptosis in the small intestinal stem cell population, but not in the more mature cells.

The small intestine was removed, fixed in Carnoy's fixative, and processed for histological analysis (paraffin embedded). One set of 3 mm sections were immunolabeled for BrdU and one set of sections were stained with H&E. Longitudinal sections of small intestinal crypts were analyzed for the presence of either BrdU or apoptoticimitotic nuclei. Fifty half crypts were scored per animal.

Groups 1-7 (Group A in the results) were tested to determine the effect of the CG53135-05 E. coli purified product over a 48 hour period. Groups 8-9 (Group B in the results) were tested to determine whether the CG53135-05 E. coli purified product changes the number of apoptotic cells generated after low dose irradiation, i.e., whether the CG53135-05 E. coli purified product influences the radiosensitive stem cell population.

The results generated show a frequency distribution for the crypts in each group of animals that were further analyzed for statistical differences. Tissue samples were harvested at 3, 6, 9,12, 24, and 48 hours after treatment with the CG53135-05 E. coli purified product. Apoptosis, mitotic index, and proliferation were the end points for this study.

Results:

Group A

In groups 1-7 (Table 40), the CG53135-05 E. coli purified product had no significant effect on spontaneous apoptosis. Similar results were obtained with the mitotic index. However, results of BrdU uptake as in Table 41, revealed the following:
a) At 3 hour, there was extension/increase of proliferative region (cell positions 12-22).
b) By 9 hours, large proliferative effects were noted in many cell positions.
c) By 12 hours, only cell positions 4-8 showed increase in uptake (stem cells).
d) By 24 hours, there was a significant inhibition of proliferation.

e) By 48 hours, the uptake was comparable to control levels.

TABLE 41
Summary of significant cell positions in the crypt after
assessment of apoptosis, mitosis, and proliferation

| Sample time | Significant Cell Positions | | |
|---|---|---|---|
| (hours) | BrdU labeling | Apoptotic | Mitotic |
| After treatment | Index | Index | Index |
| 3 | 12 to 22 | None | None |
| 6 | None | None | None |
| 9 | 5 to 9 & | None | None |
| | 11 to 20 to 21 | | |
| 12 | 4 to 8 | None | None |
| 24 | 4 to 8 | None | None |
| 48 | None | None | None |

The comparisons shown in Table 41 are between treated groups versus the untreated group. The cell positions shown are the ones that are significantly different from the untreated control (P<0.05).

Group B:

In Groups 8 and 9 (Table 40), stem cell radiosensitivity was assessed. As shown in Table 40, the CG53135-05 E. coli purified product or PBS was administered one day before dosing with 1 Gy radiation. Tissues were harvested 4.5 hours after radiation dosing. There was no significant effect of CG53135-05 administration on either radiation-induced apoptosis or mitotic index. However, increased uptake in cell positions 4-8 by 12 hours and significant inhibition of proliferation were seen in mice pretreated with CG53135-05 and irradiated, consistent with the Group A results (Table 41).

### 6.28. Example 28

### Effect of CG53135 Prophylactic Administration on Mice Intestinal Crypt Survival After Radiation Injury (Study N-343)

The purpose of this study was to evaluate the efficacy of CG53135 against radiation-induced crypt cell mortality in vivo using the Clonoquan™ assay. Protein concentrations in this example were measured by Bradford assay.

Mice were weighed and then dosed with the CG53135-05 E. coli purified product (12 mg/kg) or placebo. A single injection was given, intraperitoneally (ip), 24 hours prior to irradiation. Each group of 6 animals was irradiated as per table below. For each radiation dose, the response of a drug treated group and a placebo treated group was compared.

The small intestine was removed, fixed in Carnoy's fixative, and processed for histological analysis (paraffin embedded). H&E sections were prepared following conventional protocols. For each animal, ten intestinal circumferences were analyzed, the number of surviving crypts per circumference was scored, and the average per group was determined. Only crypts containing 10 or more strongly H&E stained cells (excluding Paneth cells) and only intact circumferences, not containing Peyers patches, were scored.

The average crypt width (measured at its widest point) was also measured in order to correct for scoring errors due to crypt size difference. The correction was applied as follows:

Corrected number of crypts per circumference=Mean number of surviving crypts per circumference in treatment group X (Mean crypt width in untreated control/Mean crypt width in treated animal).

TABLE 42
STUDY DESIGN

| Group | Number of | | | Treatment |
|---|---|---|---|---|
| Number | Animals | Induction | Treatment | Schedule* |
| 1 | 6 males | 10 Gy, Day 0 | PBS | Day −1 |
| 2 | 6 males | 11 Gy, Day 0 | PBS | Day −1 |
| 3 | 6 males | 12 Gy, Day 0 | PBS | Day −1 |
| 4 | 6 males | 13 Gy, Day 0 | PBS | Day −1 |
| 5 | 6 males | 14 Gy, Day 0 | PBS | Day −1 |
| 6 | 6 males | 10 Gy, Day 0 | CG53135-05 E. coli | Day −1 |
| | | | purified product, | |
| | | | 1 mg/kg, IP | |
| 7 | 6 males | 11 Gy, Day 0 | CG53135-05 E coli | Day −1 |
| | | | purified product, | |
| | | | 12 mg/kg, IP | |
| 8 | 6 males | 12 Gy, Day 0 | CG53135-05 E. coli | Day −1 |
| | | | purified product, | |
| | | | 12 mg/kg, IP | |
| 9 | 6 males | 13 Gy, Day 0 | CG53135-05 E. coli | Day −1 |
| | | | purified product, | |
| | | | 12 mg/kg, IP | |
| 10 | 6 males | 14 Gy, Day 0 | CG53135-05 E. coli | Day −1 |
| | | | purified product, | |
| | | | 12 mg/kg, IP | |
| 11 | 6 males | Untreated | | |

Results:

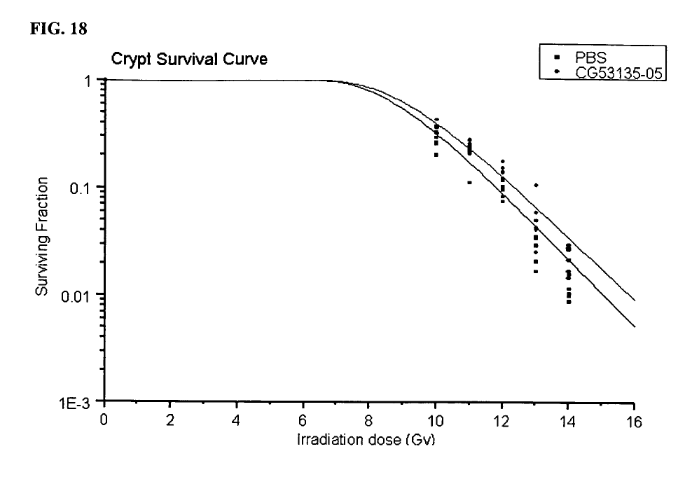

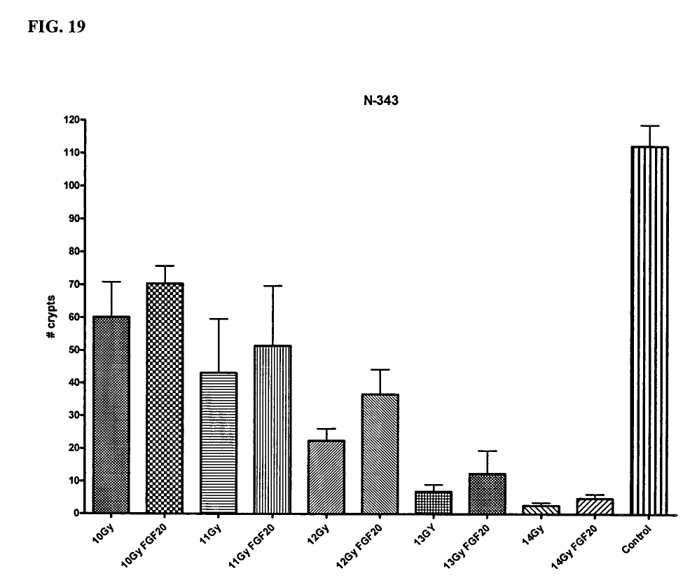

The crypt survival following prophylactic administration of the CG53135-05 E. coli purified product showed inverse correlation to the irradiation dose, that is, the smaller the radiation dose, the higher the crypt survival (FIGS. 18 and 19). Prophylactic administration of the CG53135-05 E. coli purified product significantly increased the number of crypts (P<0.001). Table 43 shows the protection factor achieved for the radiation doses following prophylactic administration of the protein (the CG53135-05 E. coli purified product). Protection factor (Table 43) represents the ratio of surviving crypt cells between treated and untreated cells. On average, 1.55 times as many cells survived irradiation dose of 12 Gy, when animals were administered with the CG53135-05 E. coli purified product prior to the radiation insult.

TABLE 43

| Radiation dose (Gy) | Protection Factor |
|---|---|
| 10 | 1.29 |
| 11 | 1.21 |
| 12 | 1.55 |
| 13 | 1.71 |
| 14 | 1.73 |

### 6.29. Example 5

### Effects of CG53135 Prophylactic Dose Schedule on Survival of Irradiated Intestinal Crypt Cells (N-375)

The objective of this study was to evaluate the ability of CG53135 to protect against radiation-indiced intestinal crypt cell mortality in vivo when administered once daily for 4 days prior to irradiation. CG53135-05 E. coli purified product (12 mg/kg) or PBS was administered to BDF1 mice intraperitoneally (IP) once daily for 4 consecutive days prior to exposure to lethal radiation doses from 10-14 Gy on Day 0. The number of surviving regenerating crypt foci was measured 4 days after irradiation. Protein concentrations in this example were measured by Bradford assay.

When animals received CG53135 once daily for 4 days, an overall increase in crypt cell survival was noted when compared to PBS-treated, irradiated animals (Table 44).

TABLE 44
Intestinal Crypt Protection Factors Resulting from
CG53135-05 E. coli purified product
Multiple-Dose Administration Prior to Irradiation

| | Mean Crypt | Mean Crypt Survival (#) | |
|---|---|---|---|
| Radiation | Survival (#) | CG53135-05 | Protection |
| Dose | PBS | (12 mg/kg) | Factor^{a} |
| 10 Gy | 32.7 | 32.2 | 0.98 |
| 11 Gy | 13.8 | 19.8 | 1.43 |
| 12 Gy | 6.6 | 8.9 | 1.35 |
| 13 Gy | 2.3 | 4.8* | 2.09 |
| 14 Gy | 1.7 | 1.3 | 0.76 |

^{a}Protection factor value indicates the number of surviving crypts per circumference in the CG53135-05-treated animals compared to PBS, expressed as a ratio.
*P ≦ 005 versus corresponding value from PBS-treated control animals by ANOVA.
# = number of crypts.

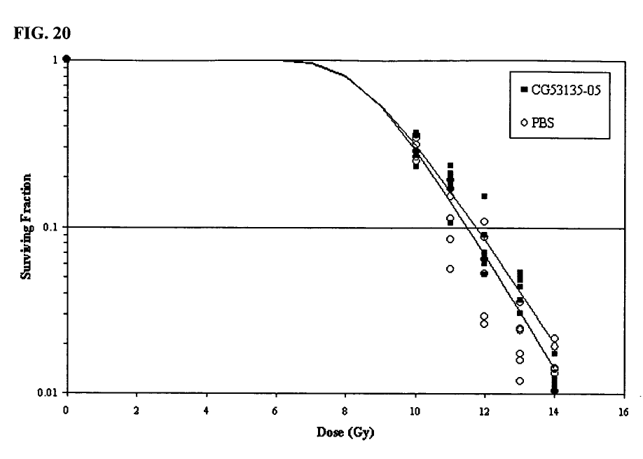

The greatest level of radioprotection occurred following 13 Gy of radiation, with a protection factor of 2.09 (e.g., a 2-fold increase in the number of surviving crypt cells). The crypt survival curves indicated a significantly reduced sensitivity to the radiation following CG53135-05 treatment (FIG. 20). Thus, pretreatment with CG53135 for 4 consecutive days increased the overall crypt cell survival. This study indicates the use of multiple-day prophylactic dosing with CG53135-05 as a schedule with radioprotective properties.

### 6.30. Example 30

### Effect of CG53135 on Repopulation of Thymus Following Bone Marrow Ablation and Subsequent Bone Marrow Transplant

Long-term effects of CG53135 specifically in the thymus microenvironment on reconstitution of the immune system were also examined. Protein concentrations in this example were measured by UV absorbance. The CG53135 E. coli purified product was tested in a bone marrow ablation and transplantation model and repopulation of the thymus with thymocytes was examined. Mice were irradiated with 9 Gy to ablate the bone marrow, and subsequently underwent bone marrow transplantation. Prior to this, one group of mice was dosed with 16 mg/kg (UV) CG53135 (IP), once daily on days −3, −2, −1, 0 and +1 relative to the day of bone marrow ablation. Thirty days after bone marrow transplantation, the thymi of both untreated and treated mice were harvested and thymocytes collected. Cells were counted (A) as well as stained (B) for the T-cell specific markers CD4 and CD8.

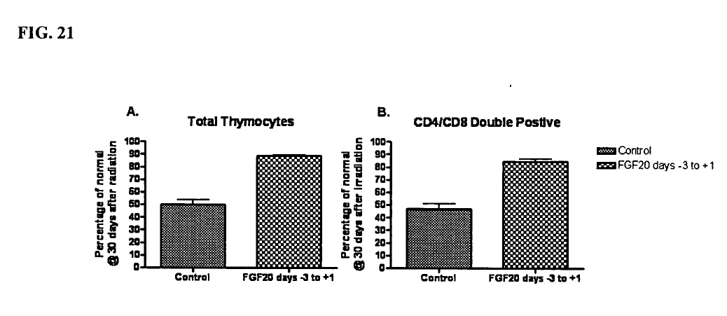

FIG. 21 shows that the total thymocyte cell population, as well as mature CD4/CD8 positive T-cells within the thymus, was significantly increased in animals treated with the CG53135-05 E. coli purified product (p=0.00003).

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

1. An isolated nucleic acid molecule selected from the group consisting of:
(a) a nucleic acid molecule comprising a nucleotide sequence selected from the group consisting of SEQ ID NOs: 1, 3, 5,6, 8, 9, 11, 13, 15, 17, 19, 21, 23, 25, 27, 29, 31, 33, 35, 37, 39 and 41;
(b) a nucleic acid molecule encoding a protein comprising an amino acid sequence selected from the group consisting of SEQ ID NOs: 2, 4, 7, 10, 12, 14, 16, 18, 20, 22, 24, 26, 28, 30, 32, 34, 36, 38, and 40;
(c) a nucleic acid molecule hybridizes under stringent conditions to a nucleotide sequence of SEQ ID NO: 1, 3, 5, 6, 8, 9, 11, 13, 15, 17, 19, 21, 23, 25, 27, 29, 31, 33, 35, 37, 39 or 41, or a complement of said nucleic acid molecule, and wherein said stringent conditions comprise a salt concentration from about 0.1 M to about 1.0 M sodium ion, a pH from about 7.0 to about 8.3, a temperature is at least about 60° C., and at least one wash in 0.2×SSC, 0.01% BSA;
(d) a fragment of an nucleic acid molecule of any of (a)-(c); and
(e) a complement of an nucleic acid molecule of any of (a)-(d).

2. The isolated nucleic acid molecule of claim 1 comprising SEQ ID NO:1.

3. The isolated nucleic acid molecule of claim 1 comprising SEQ ID NO:8.

4. The isolated nucleic acid molecule of claim 1 comprising SEQ ID NO:23.

5. A vector comprising the nucleic acid molecule of claim 1.

6. The vector of claim 5, wherein said nucleic acid molecule is operably linked to an expression control sequence.

7. A prokaryotic or eukaryotic host cell containing a nucleic acid molecule of claim 1.

8. A prokaryotic or eukaryotic host cell containing the vector of claim 5.

9. A prokaryotic or eukaryotic host cell containing the vector of claim 6.

10. A method comprising culturing the host cell of claim 8 or 9 in a suitable nutrient medium.

11. The method of claim 10, wherein said host cell is E. coli.

12. The method of claim 10 further comprising isolating a polypeptide encoded by said nucleic acid molecule from said cultured cells or said nutrient medium.

13. An isolated protein by method of claim 12.

14. An isolated protein selected from the group consisting of:
(a) a protein comprising an amino acid sequence of SEQ ID NO: 2, 4, 7, 10, 12, 14, 16, 18, 20, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NO: 2, 4, 7, 10, 12, 14, 16, 18, 20, 22, 24, 26, 28, 30, 32, 34, 36, 38, or 40, and wherein said protein with one or more amino acid substitutions retains cell proliferation stimulatory activity; and
(c) a fragment of the protein of (a) or (b).

15. The isolated protein of claim 14 comprising an amino acid sequence of SEQ ID NO:2.

16. The isolated protein of claim 14 comprising an amino acid sequence of SEQ ID NO:24.

17. An isolated polypeptide comprising an amino acid sequence, wherein said amino acid sequence has one or more conservative amino acid substitutions relative to SEQ ID NO: 2, 4, 7, 10, 12, 14, 16, 18, 20, 22, 24, 26, 28, 30, 32, 34, 36, 38, or 40.

18. An isolated polypeptide comprising an amino acid sequence, wherein said amino acid sequence is a fragment of SEQ ID NO: 2, 4, 7, 10, 22, 24, 26, 28, 30, 32, 34, 36, 38, or 40, and wherein said fragment retains cellular proliferation stimulatory activity.

19. A pharmaceutical composition comprising a pharmaceutically acceptable carrier, and a protein of any of claims 14-18.

20. A method of preventing or treating a disorder associated with pathology of epithelial cells or mesenchymal cells comprising administering to a subject in need thereof an effective amount of a composition comprising an isolated protein of claim 14.

21. A method of stimulating proliferation, differentiation or migration of epithelial cells or mesenchymal cells comprising administering to a subject in need thereof an effective amount of a composition comprising an isolated protein of claim 14.

22. The method of claim 20 or 21, wherein said composition further comprising a pharmaceutically acceptable carrier.

23. The method of any of claims 20 or 21, wherein said epithelial cells or mesenchymal cells locate at the alimentary tract of said subject.

24. The method of claim 20 or 21, wherein said epithelial cells or mesenchymal cells locate at the pulmonary tract of said subject.

25. The method of claim 24, wherein said epithelial cells or mesenchymal cells locate at trachea.

26. The method of claims 20 or 21, wherein said subject is a mammal.

27. The method of claim 26, wherein said mammal is a human.


SEQUENCE LISTING (65 sequences)

SEQ ID NO: 1 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 4 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 5 - DNA, 636 bp, Homo sapiens
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 6 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 7 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 8 - DNA, 636 bp, Homo sapiens
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 9 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 10 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 11 - DNA, 54 bp, Homo sapiens
  CDS (1)..(54)
atggctcccttagccgaagtcgggggctttctgggcggcctggagggcttgggc

SEQ ID NO: 12 - protein, 18 aa, Homo sapiens
MAPLAEVGGFLGGLEGLG

SEQ ID NO: 13 - DNA, 63 bp, Homo sapiens
  CDS (1)..(63)
gagcggccgccgctgctgggcgagcgcaggagcgcggcggagcggagcgcgcgcggcgggccg

SEQ ID NO: 14 - protein, 21 aa, Homo sapiens
ERPPLLGERRSAAERSARGGP

SEQ ID NO: 15 - DNA, 63 bp, Homo sapiens
  CDS (1)..(63)
cgcaggtattttgtggcacttaacaaagacggaactccaagagatggcgccaggtccaagagg

SEQ ID NO: 16 - protein, 21 aa, Homo sapiens
RRYFVALNKDGTPRDGARSKR

SEQ ID NO: 17 - DNA, 60 bp, Homo sapiens
  CDS (1)..(60)
cctagaccagtggatccagaaagagttccagaattgtacaaggacctactgatgtacact

SEQ ID NO: 18 - protein, 20 aa, Homo sapiens
PRPVDPERVPELYKDLLMYT

SEQ ID NO: 19 - DNA, 51 bp, Homo sapiens
  CDS (1)..(51)
atgaacgacaagggcgagctgtacggcagcgagaagctgaccagcgagtgc

SEQ ID NO: 20 - protein, 17 aa, Homo sapiens
MNDKGELYGSEKLTSEC

SEQ ID NO: 21 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 22 - protein, 211 aa, Homo sapiens [sequence omitted]

SEQ ID NO: 23 - DNA, 630 bp, Homo sapiens
  CDS (1)..(627)
ccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 24 - protein, 209 aa, Homo sapiens
PLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 612 bp, Homo sapiens
  CDS (1)..(609)
ggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 26 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 27 - DNA, 603 bp, Homo sapiens
  CDS (1)..(600)
ggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 28 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 29 - DNA, 594 bp, Homo sapiens
  CDS (1)..(591)
gagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 30 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 31 - DNA, 567 bp, Homo sapiens
  CDS (1)..(564)
cacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 32 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 33 - DNA, 402 bp, Homo sapiens
  CDS (1)..(402)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgac

SEQ ID NO: 34 - protein, 134 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVD

SEQ ID NO: 35 - DNA, 447 bp, Homo sapiens
  CDS (1)..(447)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 36 - protein, 149 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 37 - DNA, 396 bp, Homo sapiens
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagacca

SEQ ID NO: 38 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRP

SEQ ID NO: 39 - DNA, 396 bp, Homo sapiens
  CDS (1)..(396)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagacca

SEQ ID NO: 40 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRP

SEQ ID NO: 41 - DNA, 537 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 42 - DNA, 38 bp, Artificial Sequence
  Description of Artifical Sequence Primer/Probe
ctcgtcagatctccaccatggctcccttagccgaagtc

SEQ ID NO: 43 - DNA, 34 bp, Artificial Sequence
  Description of Artifical Sequence Primer/Probe
ctcgtcctcgagagtgtacatcagtaggtccttg

SEQ ID NO: 44 - DNA, 30 bp, Artificial Sequence
  Description of Artifical Sequence Primer/Probe
ctcgtcctcgagggtaagcctatccctaac

SEQ ID NO: 45 - DNA, 31 bp, Artificial Sequence
  Description of Artifical Sequence Primer/Probe
ctcgtcgggcccctgatcagcgggtttaaac

SEQ ID NO: 46 - DNA, 14 bp, Artificial Sequence
  Description of Artifical Sequence Primer/Probe
catggtcagcctac

SEQ ID NO: 47 - DNA, 14 bp, Artificial Sequence
  Description of Artifical Sequence Primer/Probe
tcgagtaggctgac

SEQ ID NO: 48 - DNA, 20 bp, Artificial Sequence
  Description of Artifical Sequence Primer/Probe
ggaccacagcctcttcggta

SEQ ID NO: 49 - DNA, 25 bp, Artificial Sequence
  Description of Artifical Sequence Primer/Probe
tgtccacacctctaatactgaccag

SEQ ID NO: 50 - DNA, 26 bp, Artificial Sequence
  Description of Artifical Sequence Primer/Probe
cccactgccacactgatgaattccaa

SEQ ID NO: 51 - DNA, 21 bp, Artificial Sequence
  Description of Artifical Sequence Primer/Probe
aggcagaagcgggagatagat

SEQ ID NO: 52 - DNA, 24 bp, Artificial Sequence
  Description of Artifical Sequence Primer/Probe
agcagctttacctcattcacaatg

SEQ ID NO: 53 - DNA, 28 bp, Artificial Sequence
  Description of Artifical Sequence Primer/Probe
ccatctacatccaccaccagttgcagaa

SEQ ID NO: 54 - DNA, 23 bp, Artificial Sequence
  Description of Artifical Sequence Primer/Probe
aggtcaccatggctgttattggc

SEQ ID NO: 55 - DNA, 29 bp, Artificial Sequence
  Description of Artifical Sequence Primer/Probe
ctgtctgtcctcagaagaagttcttgatc

SEQ ID NO: 56 - DNA, 37 bp, Artificial Sequence
  Description of Artifical Sequence Primer/Probe
caccagatctatggctcccttagccgaagtcgggggc

SEQ ID NO: 57 - DNA, 39 bp, Artificial Sequence
  Description of Artifical Sequence Primer/Probe
gccgtcgacagtgtacatcagtaggtccttgtacaattc

SEQ ID NO: 58 - DNA, 25 bp, Artificial Sequence
  Description of Artifical Sequence Primer/Probe
gtatcttggaattcatcagtgtggc

SEQ ID NO: 59 - DNA, 25 bp, Artificial Sequence
  Description of Artifical Sequence Primer/Probe
tggtctctatcttggaatgaatgac

SEQ ID NO: 60 - DNA, 20 bp, Artificial Sequence
  Description of Artifical Sequence Primer/Probe
gaagaggctgtggtcctgcc

SEQ ID NO: 61 - DNA, 25 bp, Artificial Sequence
  Description of Artifical Sequence Primer/Probe
actgtccacacctctaatactgacc

SEQ ID NO: 62 - DNA, 38 bp, Artificial Sequence
  Description of Artifical Sequence Primer/Probe
caccagatctatcctgcgccgccggcagctctattgcc

SEQ ID NO: 63 - DNA, 44 bp, Artificial Sequence
  Description of Artifical Sequence Primer/Probe
gccgtcgactggtctaggtaagaaatgtgtaaatttctgatgcc

SEQ ID NO: 64 - DNA, 38 bp, Artificial Sequence; identical to SEQ ID NO: 62
  Description of Artifical Sequence Primer/Probe

SEQ ID NO: 65 - DNA, 39 bp, Artificial Sequence; identical to SEQ ID NO: 57
  Description of Artifical Sequence Primer/Probe